SEQUENCE LISTING (52 sequences)

SEQ ID NO: 1 - DNA, 8443 bp, Homo sapiens
atgggggagctacccaaagcctggggaaggggagtcagtgctggaggtgactggagccgcggcgacggcggcagcagctgcggtggcggcgctctcagccgtggcagccgaggctgcagcccggcccagggcggctcagcggacaataaccgggctaggaacctgaccgggctggaccccactgtgatgcttcctgactgataatgttataacagtgcctggaagcctgaggctgcatttccagctggacgtacctcagttgtccccatcatcatggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagcccctcaggtgccctgctttgaggagaccttccaagcccctagaaaggaaaatgctaacccccaagatgcccctgggccaaaagtttctccaacaccagaaccttctccaccagctactgagaaaatggcctgtaaagatccagaaaaacccatggaggcctgtgcctcagcacatgtgcaacccaagcctgcccctgaagccagtagcctagaggagccccatagcccagaaacaggggagaaggtagtagcaggagaggtaaacccacccaatggccctgtgggggacccactgagcctcttgtttggggatgtgacatccctgaaaagctttgattcattgacaggttgtggtgacataatagcagaacaggacatggacagtatgacagacagcatggcctctgggggccagagagcaaaccgagatgggaccaagcgaagttcctgcctggtgacctaccaaggaggtggggaggagatggccttgccagatgatgatgacgaggaggaagaagaggaagaagaggtggaattagaggaggaagaagaggaggttaaggaggaggaagaagatgatgacttagaatatctgtgggaaactgcccaaatgtatccacggcccaatatgaacctgggctaccatcccaccacatccccaggccaccacggctacatgctccttgacccagttaggtcttatcctggcctagcccctggggaacttttgactcctcagagtgaccagcaagaatccgcccccaatagtgatgaaggttattatgactccaccacacctggatttgaggatgattcaggtgaggccctggggcttgtccgcagggattgtctaccccgagacagctacagtggagatgccctatatgagttctatgagccagatgacagccttgagaactctccacctggagatgactgcctttatgacctccatggtcgaagctctgagatgtttgaccccttcttaaactttgagccctttttgtcctcccggccacctggggcaatggagacagaggaagaacggctagtgaccatccagaaacagttgttgtattgggagcttcggcgggagcagcttgaggcccaggaggcacgtgcccgagaagctcatgccagggaggcccacgccagggaggcctatactcgagaggcttatggcagggaagcctatgccagggaggcccacacttgggaagctcatggcagggaggccagaacccgagaagcccaggcccgagaggttcgttgtagagagactcaagtccgagagacccaggcccggcaggagaagcccgtcttagagtatcagatgaggcccttaggcccatcagtgatgggcctggcagcaggggtatcagggacctctcagatttcccaccggggaattacctcagctttccccaccactgcaagcagcgagccagactggagggacttccgtcctctggagaagcgttatgaaggaacctgctccaagaaagatcaaagtacctgcctgatgcagctcttccagagtgatgccatgtttgagccagacatgcaagaagcaaattttggaggatctcccaggagggcctaccctacttattcaccccctgaagatccagaggaagaggaggttgagaaggaagggaatgccactgtgagtttctcacaggccctggtagagttcaccagcaatgggaacctcttttccagcatgtcctgcagctctgactctgactcatctttcactcaaaacctccctgagctgcctcccatggtgacctttgacatcgctgatgtggaacgggatggggaaggcaagtgtgaagagaatcctgagttccacaatgatgaagatcttgcagcctccttggaagcctttgagctgggctactatcacaaacatgccttcaacaactaccatagtcgattctaccaaggcctgccctggggtgtgagcagcctccctcgatacttgggactgcctggcctgcaccctcgacctccacctgctgctatggccctcaacaggagaagccgctctctcgacactgcagagaccctggagatggagctctccaattcccacttggtccagggctacctcgagtctgatgagctgcaggcccagcaggaagattcagatgaggaggacgaagaggaggaagaaggagaatggagccgagacagtcccctttccctctatactgaacccccaggggcctatgattggcctgcttgggctccctgtcctcttccagtggggccaggtcctgcctggataagccccaaccagttggacaggccttccagccagtctccatataggcaggcaacctgttgcatacctcctatgaccatgtcaatatcactatcagtgccagagtcaagggcacctggggaatctgggcctcaactagctcgtccctcacacctacacctgcccatgggcccttgctataacctccagccacaggcctcccagagcatgagggccaggcctcgagatgtgctgctgcctgttgatgagcccagttgctcttccagttctggaggcttcagccccagccctctaccacaggccaagcctgtgggcatcacccatggcattcctcagctgcccagggtccggcctgagcacccccagcctcagcccactcactatgggccttccagccttgacctgtcaaaggagagggctgagcaaggtgcctctcttgccaccagctactcctccactgccatgaatggaaacctagccaagtagttattatcaattctggagtagggacacggggcctgagcatgtgaatggggatcaggggttgggcagaagggtgggggggtgggggttgttgtttaacttgaaaaccctttgggccaaggaaaactcctgtgagttttcaccttggttttcccacttgcctcttctgccatctatggccacgttcaactcaagtgcatctgcccagggaggctgctgctgctactgcactccagtttttgctttcgaggtttcttcttgtgacccacgctgggtttgggatgctgtaattttgtgcctattccagttgccaagttagtgattatatatatatatatatatatatatatatatatatatatatatacacacacacacacacacacacacacacacatacatacacacacacacacaaacacacatgcacacacatatatagcatgcatccttgacacaggctaaacctttagtgccccctgcccctttcccccaatgaatgatgaaccactgattagcagccaagtctgcattgcctcccgcttggggaagtggggcttatttgctattagcaatgtgaaattatggtacagatcctccccacccctattccttttggcctggcatagctgccatcattatatcagatgactataagctattcatcccagctagctgtccttagagaggatcaagtagcgtgtgtctcacagcctatcgctgtggactttggcatcatgaccagatgggggccttttaactgtcctgccatagcttgggttaaagacaaaaatgctaatccccttgatggagtgataaaattcatccatttgcaaacagaagacaaaacaaggttctgttgtggacactgccatagagtgttgagctccggaatgataggattttttttccttcttttaagcttgtgactacctgagttgcagatgcttttgaatttttgttcttttttcctctaaaggaaattttcataatctcatcacaaggaacacggtcttttaattgagataagggaagagaagcaacctaaattaggcttgactataggggctggggtttttgtggagtgtaggggaaagatagaaaatgagggtccatttatcacagccagcaagaaaggggatttcagaccatgcagagcttaaggtatctcagtttcttaaaagggaaaactcccttctcaagcctcccaacacaaaatgttccttaagccttgcccatcccatcatttggactctgtaataaggccccaggtttgcactcccaagacaggataccaaaggcatctcagctgctgttctgggccctttaaagagcttttatcttccaattagccgagtccttagcatcatcaaatgacagggcttcttggcagttgcagccctttatggaatgaatggcaccctcccttctcctgcatccatgcagttttcatgttaaactaggttggaaatactggtggggaagagcttgctgtcaggaagacattggaacaggctaatcttgccaagctcaacctctcattaatcacctgtaggagagggttccttttacatgtctttattggaaatggcatatggaacattttttgcttggctgtgttcactcaaattatgattattgcaccctcgccctcacatcccaccatctccatgcctgttgagagaagaagtcaacagggttcttgccattcctgagatatggcaatttagcttgggaacagatgctgtttgccccctaagtcaaggtgactttgtgggcaaactgtttcttggccaggggatgtgtccatgctggtctttgtcaccatttctcagattggggttggcccagtgcccttactcacaaatagcagacctggctggtagaagctgcacccctggaagcaccacatgaatttgcatggcactaacaccaccactcaagggacccagaccagctcttcaggctgactttggctacagatacattgtgaagaaatgtatctgttgtggagctggagaatgaaagagggtgaggaaggccctccttaccccaagcacagcattccaagagagaagtgcccaaattccaactttctggcctcttaccaaagctgctctgggatggtggggctgaaattggcctggctatttggcaggaagtaaacactcaccaaatgcctttacaacagggaagaacccacctcttgcctcttctttcagggcagcttgagctcctgtctacatgtggactgtgccagccacagcaggcggtagagccatcaccagcctccagaccctgctggagaagggccaccagcctatctgggaagagaaaaaggaggtggccaacctttggctaagggctgagagctgccctcaggatcaggaaggcagcatgaggcattgtctctgaggggcactggcatagccctgacacactttgttgggctttggaaggtcacacaccacagagtagggcctggggctgagtgagagttcatggtttggacagccttgctgcctgcaagtcccttcactgccctagccagcccaggaataggatccttaccacctgaagtggggcagccaggatatgtggctgcagaagcaggttcctttgaatttctagagctaagcagtttgtggctgaaacccaatggcagcctaggggaggaaggaagacctgtctggggacccatggtgttgacagcacagggtgacattgggagagccagccaagcttcaggcctgtttccaaacacagactagtgctggcttgggtggcagcaaatacaccccagaccactaaaactgttgatatcatgtccacacctccaagagtactactagatgaacaacatgtctccattgcaaggtcctggaataatgcttttggatgtgattttaagggaaaatgggacttagtggaagtagctgccttcatcttattacccaataggcctagtgccagacagctgtgatcctgagaattcccagatgagactggtccttggaggaccagtgaatgtggatagagaaggccacctagtaggcctctcccagggccaggatgccagtttcttggtgctttagagtctccaaggaagcactgccttggggcccagttcaagccagccctacctatctggcctcagcttgggtaccaggccacaagactgcccggataatgggttcagtaaacaccagtctctctccctccttagtcctatgccaagaagggaagtagcttcacacactcaggaggctgggtgtgtacccttcatcctgctgccagcagctttccttacctgtgccagtgccaccaccccacctgccctcagcccccaggcccagttgctagagggaggcatggggtttagcagggagacttcaagtttacccatgtgtatgttgcattcaagttgtttttaatatatatatatatatatatcaaaagtcaattatctatttttatattgtttgcattttatattcatggaaaacaatgtttatcaattgttcttttctgatttatttttaagcggtgctgtttacagttttaaacaaaacaaagatgacacaaaaattgctgctgcttgtgcagagggctggcatctcaactgcctagggccctccctctttcccctctccctaccttagctgtaccacactgtccgttccctccccccatgatagggacagcagaagatgcccaggtactgaagacaaacgccaagtgactcaggcgtgtgggacctggtagacagcttgtcattttctggctttattttatgtttttctttcttttgaacccttcttccccatgtctagaccctctgagagtactcatgtgtcttattttttttcctcgttatgttttgtgctttagttttgaaatcagagtgcccatgttgtgctatttgtcttgttgagtttctccaaaggagcattttgggaacattctccacccaatggggaaggaagggggtttcaaccatgtcccctttccccaagaagctgtgggagacagatgacaatgatgcttgcactatcaggaatttaccatctcgttgaggaaacccttgcataggacttcctaggagctcagttgattgtgacattttcccagaagagaggctttggggaactccttcacccctcttgctgctttgggggaatctcctcccctaccaccattccccctcccctgccctgctccagctcacacgtggatggtactttggggaacagtgactttcagggtgatggcttgctggcctgatccctgactttccaatcctatagtcactccaaagaagaaaatccacatggtatgtatatgtgtgagggttgggtctattgagaggtgggactttaattattgttattattattggtgcttatttttcctgctgtatatgggggtgggggtgggggggaataaaacaggaatgtatatttaggtcatgttaaataaaatggctggaggaaggagggaagaaattaggaactgcagtacatcatcaccccaggtgtaagagttatttctctcccagtgcagaaaggggagtgtggcccctggctgtacctgagcttcctcttctccatccctgagacatcacagaatcacccgggttctgacctctccacttctagctctcactgacatctggatatcttgatgcactgcctgtttgtaacaacatatacttctttgctgaatgctaataaacttctgggttcaagagggaacccctcagtca

SEQ ID NO: 2 - protein, 1135 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAPQVPCFEETFQAPRKENANPQDAPGPKVSPTPEPSPPATEKMACKDPEKPMEACASAHVQPKPAPEASSLEEPHSPETGEKVVAGEVNPPNGPVGDPLSLLFGDVTSLKSFDSLTGCGDIIAEQDMDSMTDSMASGGQRANRDGTKRSSCLVTYQGGGEEMALPDDDDEEEEEEEEVELEEEEEEVKEEEEDDDLEYLWETAQMYPRPNMNLGYHPTTSPGHHGYMLLDPVRSYPGLAPGELLTPQSDQQESAPNSDEGYYDSTTPGFEDDSGEALGLVRRDCLPRDSYSGDALYEFYEPDDSLENSPPGDDCLYDLHGRSSEMFDPFLNFEPFLSSRPPGAMETEEERLVTIQKQLLYWELRREQLEAQEARAREAHAREAHAREAYTREAYGREAYAREAHTWEAHGREARTREAQAREVRCRETQVRETQARQEKPVLEYQMRPLGPSVMGLAAGVSGTSQISHRGITSAFPTTASSEPDWRDFRPLEKRYEGTCSKKDQSTCLMQLFQSDAMFEPDMQEANFGGSPRRAYPTYSPPEDPEEEEVEKEGNATVSFSQALVEFTSNGNLFSSMSCSSDSDSSFTQNLPELPPMVTFDIADVERDGEGKCEENPEFHNDEDLAASLEAFELGYYHKHAFNNYHSRFYQGLPWGVSSLPRYLGLPGLHPRPPPAAMALNRRSRSLDTAETLEMELSNSHLVQGYLESDELQAQQEDSDEEDEEEEEGEWSRDSPLSLYTEPPGAYDWPAWAPCPLPVGPGPAWISPNQLDRPSSQSPYRQATCCIPPMTMSISLSVPESRAPGESGPQLARPSHLHLPMGPCYNLQPQASQSMRARPRDVLLPVDEPSCSSSSGGFSPSPLPQAKPVGITHGIPQLPRVRPEHPQPQPTHYGPSSLDLSKERAEQGASLATSYSSTAMNGNLAK

SEQ ID NO: 3 - DNA, 2577 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggttgtggtgacataatagcagaacaggacatggacagtatgacagacagcatggcctctgggggccagagagcaaaccgagatgggaccaagcgaagttcctgcctggtgacctaccaaggaggtggggaggagatggccttgccagatgatgatgacgaggaggaagaagaggaagaagaggtggaattagaggaggaagaagaggaggttaaggaggaggaagaagatgatgacttagaatatctgtgggaaactgcccaaatgtatccacggcccaatatgaacctgggctaccatcccaccacatccccaggccaccacggctacatgctccttgacccagttaggtcttatcctggcctagcccctggggaacttttgactcctcagagtgaccagcaagaatccgcccccaatagtgatgaaggttattatgactccaccacacctggatttgaggatgattcaggtgaggccctggggcttgtccgcagggattgtctaccccgagacagctacagtggagatgccctatatgagttctatgagccagatgacagccttgagaactctccacctggagatgactgcctttatgacctccatggtcgaagctctgagatgtttgaccccttcttaaactttgagccctttttgtcctcccggccacctggggcaatggagacagaggaagaacggctagtgaccatccagaaacagttgttgtattgggagcttcggcgggagcagcttgaggcccaggaggcacgtgcccgagaagctcatgccagggaggcccacgccagggaggcctatactcgagaggcttatggcagggaagcctatgccagggaggcccacacttgggaagctcatggcagggaggccagaacccgagaagcccaggcccgagaggttcgttgtagagagactcaagtccgagagacccaggcccggcaggagaagcccgtcttagagtatcagatgaggcccttaggcccatcagtgatgggcctggcagcaggggtatcagggacctctcagatttcccaccggggaattacctcagctttccccaccactgcaagcagcgagccagactggagggacttccgtcctctggagaagcgttatgaaggaacctgctccaagaaagatcaaagtacctgcctgatgcagctcttccagagtgatgccatgtttgagccagacatgcaagaagcaaattttggaggatctcccaggagggcctaccctacttattcaccccctgaagatccagaggaagaggaggttgagaaggaagggaatgccactgtgagtttctcacaggccctggtagagttcaccagcaatgggaacctcttttccagcatgtcctgcagctctgactctgactcatctttcactcaaaacctccctgagctgcctcccatggtgacctttgacatcgctgatgtggaacgggatggggaaggcaagtgtgaagagaatcctgagttccacaatgatgaagatcttgcagcctccttggaagcctttgagctgggctactatcacaaacatgccttcaacaactaccatagtcgattctaccaaggcctgccctggggtgtgagcagcctccctcgatacttgggactgcctggcctgcaccctcgacctccacctgctgctatggccctcaacaggagaagccgctctctcgacactgcagagaccctggagatggagctctccaattcccacttggtccagggctacctcgagtctgatgagctgcaggcccagcaggaagattcagatgaggaggacgaagaggaggaagaaggagaatggagccgagacagtcccctttccctctatactgaacccccaggggcctatgattggcctgcttgggctccctgtcctcttccagtggggccaggtcctgcctggataagccccaaccagttggacaggccttccagccagtctccatataggcaggcaacctgttgcatacctcctatgaccatgtcaatatcactatcagtgccagagtcaagggcacctggggaatctgggcctcaactagctcgtccctcacacctacacctgcccatgggcccttgctataacctccagccacaggcctcccagagcatgagggccaggcctcgagatgtgctgctgcctgttgatgagcccagttgctcttccagttctggaggcttcagccccagccctctaccacaggccaagcctgtgggcatcacccatggcattcctcagctgcccagggtccggcctgagcacccccagcctcagcccactcactatgggccttccagccttgacctgtcaaaggagagggctgagcaaggtgcctctcttgccaccagctactcctccactgccatgaatggaaacctagccaagtag

SEQ ID NO: 4 - protein, 858 aa, Homo sapiens
METQKDEAAQAKGAAASGSTREQTAEKGAKNKAAEATEGPTSEPSSSGPGCGDIIAEQDMDSMTDSMASGGQRANRDGTKRSSCLVTYQGGGEEMALPDDDDEEEEEEEEVELEEEEEEVKEEEEDDDLEYLWETAQMYPRPNMNLGYHPTTSPGHHGYMLLDPVRSYPGLAPGELLTPQSDQQESAPNSDEGYYDSTTPGFEDDSGEALGLVRRDCLPRDSYSGDALYEFYEPDDSLENSPPGDDCLYDLHGRSSEMFDPFLNFEPFLSSRPPGAMETEEERLVTIQKQLLYWELRREQLEAQEARAREAHAREAHAREAYTREAYGREAYAREAHTWEAHGREARTREAQAREVRCRETQVRETQARQEKPVLEYQMRPLGPSVMGLAAGVSGTSQISHRGITSAFPTTASSEPDWRDFRPLEKRYEGTCSKKDQSTCLMQLFQSDAMFEPDMQEANFGGSPRRAYPTYSPPEDPEEEEVEKEGNATVSFSQALVEFTSNGNLFSSMSCSSDSDSSFTQNLPELPPMVTFDIADVERDGEGKCEENPEFHNDEDLAASLEAFELGYYHKHAFNNYHSRFYQGLPWGVSSLPRYLGLPGLHPRPPPAAMALNRRSRSLDTAETLEMELSNSHLVQGYLESDELQAQQEDSDEEDEEEEEGEWSRDSPLSLYTEPPGAYDWPAWAPCPLPVGPGPAWISPNQLDRPSSQSPYRQATCCIPPMTMSISLSVPESRAPGESGPQLARPSHLHLPMGPCYNLQPQASQSMRARPRDVLLPVDEPSCSSSSGGFSPSPLPQAKPVGITHGIPQLPRVRPEHPQPQPTHYGPSSLDLSKERAEQGASLATSYSSTAMNGNLAK

SEQ ID NO: 5 - DNA, 843 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagcccctcaggtgccctgctttgaggagaccttccaagccctagaaaggaaaatgctaacccccaagatgcccctgggccaaaagtttctccaacaccagaaccttctccaccagctactgagaaaatggcctgtaaagatccagaaaaacccatggaggcctgtgcctcagcacatgtgcaacccaagcctgcccctgaagccagtagcctag

SEQ ID NO: 6 - protein, 280 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAPQVPCFEETFQALERKMLTPKMPLGQKFLQHQNLLHQLLRKWPVKIQKNPWRPVPQHMCNPSLPLKPVA

SEQ ID NO: 7 - DNA, 831 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagcccctcaggtgccctgctttgaggagaccttccaagcccctagaaaggaaaatgctaacccccaagatgcccctgggccaaaagtttctccaacaccagaaccttctccaccagctactgagaaaatggcctgtaaagatccagaaaaaacccatggaggcctgtgcctcagcacatgtgcaacccaagcctgcccctga

SEQ ID NO: 8 - protein, 276 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAPQVPCFEETFQAPRKENANPQDAPGPKVSPTPEPSPPATEKMACKDPEKTHGGLCLSTCATQACP

SEQ ID NO: 9 - DNA, 1074 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagcccctcaggtgccctgctttgaggagaccttccaagcccctagaaaggaaaatgctaacccccaagatgcccctgggccaaaagtttctccaacaccagaaccttctccaccagctactgagaaaatggcctgtaaagatccagaaaaacccatggaggcctgtgcctcagcacatgtgcaacccaagcctgcccctgaagccagtagcctagaggagccccatagcccagaaacaggggagaaggtagtagcaggagaggtaaacccacccaatggccctgtgggggacccactgagcctcttgtttggggatgtgacatccctgaaaagctttgattcattgacaggttgtggtgacataatagcagaacaggacatggacagtatgacagacagcatggcctctgggggccagagagcaaaccgagatgggaccaagtga

SEQ ID NO: 10 - protein, 357 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAPQVPCFEETFQAPRKENANPQDAPGPKVSPTPEPSPPATEKMACKDPEKPMEACASAHVQPKPAPEASSLEEPHSPETGEKVVAGEVNPPNGPVGDPLSLLFGDVTSLKSFDSLTGCGDIIAEQDMDSMTDSMASGGQRANRDGTK

SEQ ID NO: 11 - DNA, 1275 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagcccctcaggtgccctgctttgaggagaccttccaagcccctagaaaggaaaatgctaacccccaagatgcccctgggccaaaagtttctccaacaccagaaccttctccaccagctactgagaaaatggcctgtaaagatccagaaaaacccatggaggcctgtgcctcagcacatgtgcaacccaagcctgcccctgaagccagtagcctagaggagccccatagcccagaaacaggggagaaggtagtagcaggagaggtaaacccacccaatggccctgtgggggacccactgagcctcttgtttggggatgtgacatccctgaaaagctttgattcattgacaggttgtggtgacataatagcagaacaggacatggacagtatgacagacagcatggcctctgggggccagagagcaaaccgagatgggaccaagcgaagttcctgcctggtgacctaccaaggaggtggggaggagatggccttgccagatgatgatgacgaggaggaagaagaggaagaagaggtggaattagaggaggaagaagaggaggttaaggaggaggaagaagatgatgacttagaatatctgtgggaaactgcccaaatgtatccacggcccaatatgaacctgggctag

SEQ ID NO: 12 - protein, 424 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAPQVPCFEETFQAPRKENANPQDAPGPKVSPTPEPSPPATEKMACKDPEKPMEACASAHVQPKPAPEASSLEEPHSPETGEKVVAGEVNPPNGPVGDPLSLLFGDVTSLKSFDSLTGCGDIIAEQDMDSMTDSMASGGQRANRDGTKRSSCLVTYQGGGEEMALPDDDDEEEEEEEEVELEEEEEEVKEEEEDDDLEYLWETAQMYPRPNMNLG

SEQ ID NO: 13 - DNA, 1620 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagcccctcaggtgccctgctttgaggagaccttccaagcccctagaaaggaaaatgctaacccccaagatgcccctgggccaaaagtttctccaacaccagaaccttctccaccagctactgagaaaatggcctgtaaagatccagaaaaacccatggaggcctgtgcctcagcacatgtgcaacccaagcctgcccctgaagccagtagcctagaggagccccatagcccagaaacaggggagaaggtagtagcaggagaggtaaacccacccaatggccctgtgggggacccactgagcctcttgtttggggatgtgacatccctgaaaagctttgattcattgacaggttgtggtgacataatagcagaacaggacatggacagtatgacagacagcatggcctctgggggccagagagcaaaccgagatgggaccaagcgaagttcctgcctggtgacctaccaaggaggtggggaggagatggccttgccagatgatgatgacgaggaggaagaagaggaagaagaggtggaattagaggaggaagaagaggaggttaaggaggaggaagaagatgatgacttagaatatctgtgggaaactgcccaaatgtatccacggcccaatatgaacctgggctaccatcccaccacatccccaggccaccacggctacatgctccttgacccagttaggtcttatcctggcctagcccctggggaacttttgactcctcagagtgaccagcaagaatccgcccccaatagtgatgaaggttattatgactccaccacacctggatttgaggatgattcaggtgaggccctggggcttgtccgcagggattgtctaccccgagacagctacagtgggatgccctatatgagttctatgagccagatgacagccttgagaactctccacctggagatgactgcctttatgacctccatggtcgaagctctgagatgtttgaccccttcttaa

SEQ ID NO: 14 - protein, 539 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAPQVPCFEETFQAPRKENANPQDAPGPKVSPTPEPSPPATEKMACKDPEKPMEACASAHVQPKPAPEASSLEEPHSPETGEKVVAGEVNPPNGPVGDPLSLLFGDVTSLKSFDSLTGCGDIIAEQDMDSMTDSMASGGQRANRDGTKRSSCLVTYQGGGEEMALPDDDDEEEEEEEEVELEEEEEEVKEEEEDDDLEYLWETAQMYPRPNMNLGYHPTTSPGHHGYMLLDPVRSYPGLAPGELLTPQSDQQESAPNSDEGYYDSTTPGFEDDSGEALGLVRRDCLPRDSYSGMPYMSSMSQMTALRTLHLEMTAFMTSMVEALRCLTPS

SEQ ID NO: 15 - DNA, 507 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaatcctaa

SEQ ID NO: 16 - protein, 168 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKS

SEQ ID NO: 17 - DNA, 639 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagccccttag

SEQ ID NO: 18 - protein, 212 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAP

SEQ ID NO: 19 - DNA, 966 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagcccctcaggtgccctgctttgaggagaccttccaagcccctagaaaggaaaatgctaacccccaagatgcccctgggccaaaagtttctccaacaccagaaccttctccaccagctactgagaaaatggcctgtaaagatccagaaaaacccatggaggcctgtgcctcagcacatgtgcaacccaagcctgcccctgaagccagtagcctagaggagccccatagcccagaaacgggagaaggtagtagcaggagaggtaaacccacccaatggccctgtgggggacccactgagcctcttgtttggggatgtgacatccctgaaaagctttga

SEQ ID NO: 20 - protein, 321 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAPQVPCFEETFQAPRKENANPQDAPGPKVSPTPEPSPPATEKMACKDPEKPMEACASAHVQPKPAPEASSLEEPHSPETGEGSSRRGKPTQWPCGGPTEPLVWGCDIPEKL

SEQ ID NO: 21 - DNA, 1059 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagcccctcaggtgccctgctttgaggagaccttccaagcccctagaaaggaaaatgctaacccccaagatgcccctgggccaaaagtttctccaacaccagaaccttctccaccagctactgagaaaatggcctgtaaagatccagaaaaacccatggaggcctgtgcctcagcacatgtgcaacccaagcctgcccctgaagccagtagcctagaggagccccatagcccagaaacaggggagaaggtagtagcaggagaggtaaacccacccaatggccctgtgggggacccactgagcctcttgtttggggatgtgacatccctgaaaagctttgattcattgacaggttgtggtgacataatagcagaacaggacatggacagtatgacagacagcatggcctctgggggccagagagcaaactga

SEQ ID NO: 22 - protein, 352 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAPQVPCFEETFQAPRKENANPQDAPGPKVSPTPEPSPPATEKMACKDPEKPMEACASAHVQPKPAPEASSLEEPHSPETGEKVVAGEVNPPNGPVGDPLSLLFGDVTSLKSFDSLTGCGDIIAEQDMDSMTDSMASGGQRAN

SEQ ID NO: 23 - DNA, 1737 bp, Homo sapiens
atggagacccaaaaggatgaagctgctcaggccaagggagctgcagcctctgggagtacccgtgaacaaacagcagaaaaaggagccaagaacaaggcagctgaggcgacagaaggaccaacctcagagccatcctcatccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaagtttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacgtgagctcagcccctcaggtgccctgctttgaggagaccttccaagcccctagaaaggaaaatgctaacccccaagatgcccctgggccaaaagtttctccaacaccagaaccttctccaccagctactgagaaaatggcctgtaaagatccagaaaaacccatggaggcctgtgcctcagcacatgtgcaacccaagcctgcccctgaagccagtagcctagaggagccccatagcccagaaacaggggagaaggtagtagcaggagaggtaaacccacccaatggccctgtgggggacccactgagcctcttgtttggggatgtgacatccctgaaaagctttgattcattgacaggttgtggtgacataatagcagaacaggacatggacagtatgacagacagcatggcctctgggggccagagagcaaaccgagatgggaccaagcgaagttcctgcctggtgacctaccaaggaggtggggaggagatggccttgccagatgatgatgacgaggaggaagaagaggaagaagaggtggaattagaggaggaagaagaggaggttaaggaggaggaagaagatgatgacttagaatatctgtgggaaactgcccaaatgtatccacggcccaatatgaacctgggctaccatcccaccacatccccaggccaccacggctacatgctccttgacccagttaggtcttatcctggcctagcccctggggaacttttgactcctcagagtgaccagcaagaatccgcccccaatagtgatgaaggttattatgactccaccacacctggatttgaggatgattcaggtgaggccctggggcttgtccgcagggattgtctaccccgagacagctacagtggagatgccctatatgagttctatgagccagatgacagccttgagaactctccacctggagatgactgcctttatgacctccatggtcgaagctctgagatgtttgaccccttcttaaactttgagcccttttttgtcctcccggccacctggggcaatggagacagaggaagaacggctagtgaccatccagaaacagttgttgtattgggagcttcggcgggagcagcttga

SEQ ID NO: 24 - protein, 578 aa, Homo sapiens
METQKDEAAQAKGAAASGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKFPSMPKPKKGLKGFFSSIRRHRKSKVTGAEQSEPGAKGPERVRARPHEHVSSAPQVPCFEETFQAPRKENANPQDAPGPKVSPTPEPSPPATEKMACKDPEKPMEACASAHVQPKPAPEASSLEEPHSPETGEKVVAGEVNPPNGPVGDPLSLLFGDVTSLKSFDSLTGCGDIIAEQDMDSMTDSMASGGQRANRDGTKRSSCLVTYQGGGEEMALPDDDDEEEEEEEEVELEEEEEEVKEEEEDDDLEYLWETAQMYPRPNMNLGYHPTTSPGHHGYMLLDPVRSYPGLAPGELLTPQSDQQESAPNSDEGYYDSTTPGFEDDSGEALGLVRRDCLPRDSYSGDALYEFYEPDDSLENSPPGDDCLYDLHGRSSEMFDPFLNFEPFFVLPATWGNGDRGRTASDHPETVVVLGASAGAA

SEQ ID NO: 25 - DNA, 8448 bp, Mus musculus
atgggggagctgcccagagcctggagaagggtagtcggcgcagaagactgctgtagtcccggtgaaggctgcggcaggagcagtggtggcgttctcagcagaggctccagctcaaatcggagcagcgcagcagacaataaacagcgctgggagctggacctggcaggaccccactgtgatgcttcctgactgatcatgttataagtgtgcccggaatccaaaggcagcatatccagtggaagaattgtcttcatcatggagagtcaacaggatgaagctgttcaaaccaaaggagcatcaacttctagcgatgcccaagaccagggagctgaaaagggagccaagaataagacaactgaggcaacagaaggaccaacatcagagcctcccttatcaggcccaggtaggctgaagaaaactgccatgaaactctttggtggcaagaagggaatctgtactctgcctagtttctttgggggtggacgaagcaaaggttctgggaaagtcagctctaagaaaagtctaaacaagagtaagactcacgatggtctaagtgaagcgtctcagggacctgaagatgttgtcattgaagaaacagacctctctacacctttgtccaagtcatctgctcaatttcctagctcccagagtgccaatggagcgttggagataggctctaaacataagacatctgggactgaagccatagaaaaagctggagttgagaaggtaccctctgtgcacaagccgaagaaaagcctaaaaagcttctttagcagtatccgccgtcaccggaagggcaagacttctggggctgaccaaagtgtgccaggggccaaggaacttgagggggcaagaaccaggtctcatgagcatgtgagttcaatctctctgcccagctctgaggagatattccgagacactagaaaggaaaatgccaaaccccaggatgcccctggaccaaaaatgtctccagcacaagtacatttttcaccaactactgagaaggcagcttgtaaaaatccagaaaaactcacaaggacctgtgcctcagagttcatgcagcccaagcctgttcttgaaggtggtagtctagaggagccccacacttcagaaacagaaggaaaagtggtggcaggagaagtaaacccacccaatggccctgttggggatcagctaagcctcttgtttggggatgttacatcgctaaaaagctttgactcactgacaggttgtggtgacattatagcagaacaggacatggacagcatgacagatagcatggcctctggtggccagagggccaaccgagatgggaccaagcgaagttcctgcttggtaacctaccaaggaggtggggaggagatggccttgccagatgatgatgacaatgatgatgaagaagaagaagaagaagaagaagaagaagaagaagaagaagaagaagaagaagaagaagaagaagaagaagaagaagaagaattattagaggatgaagaggaggttaaggatggagaggaaaatgatgacttagagtatctgtgggcaagtgcccagatatacccaagatttaatatgaatctgggctaccatactgctatatctcccagccaccagggctacatgcttcttgacccagttcagtcttatcctaatctaggccttggagaacttttgacacctcagagcgaccagcaagagtctgctcccaatagtgatgaaggttattatgactctaccacaccaggatttgaggatgattcaggtgaggctctgggacttgcccacagggattgtctacccagggacagctatagtggtgatgcgctatatgagttctatgagccagatgacagtcttgagcactccccacctggggatgactgcctatatgacctccgtggacgcaactcagagatgttggatcctttcttgaacttggagcccttttcttctcggccacctggggcaatggaaaccgaggaagaacggctagtgaccatccagaagcagttgctatactgggaacttcgccgggagcagcgtgaggctcaagaggcatgtgccagagaggctcatgccagagaagcttatgcccgagacacccatactcgggaatcttatggcagaaatgtccgagcccgagaaactcaggccttagaggctcattctcaagagggtcgagtccaagagaccaaggtacggcaggagaagcctgccttggagtatcagatgaggcccttaggtccctcagtgatgggtctggtggcagggacatcaggaggttctcagacttctcaccggggaaccacttcagctttccctgctacttctagcagtgagccagactggagggatttccgtcctctggagaagcgttttgaggggacttgttccaaaaaagatcaaagtacttgtttaatgcagctcttccagagtgatgccatgtttgagccagacatgcaagaagcaaattttggaggctcacccaggaaggcgtaccctagttactcaccccctgaggagccagaggaagaagaagaggagaaggaagggaatgccactgtgagtttctcacaggccctagtagagttcaccagcaatggaaacctttttaccagcatgtcctacagctctgattctgactcatccttcactcaaaacctccctgagctgccccccatggtcacctttgatattgctgacgtggaacgagatggagaaggcaagtgtgaagagaatcctgagttcaacaatgacgaagatcttacagcctccttggaagcttttgagctgggttattaccacaaacatgccttcaacagctatcacagtcggttctaccaaggcctgccctggggagttagcagcctccctcgatacttaggactccccggagtccaccctcgacctcctcctgctgcaatggcactcaataggagaagccgttcccttgacaatgcagagtctctggagctggagctctccagttctcacctagcccagggttacatggagtctgatgagctccaggcccaccaggaagattcagatgaagaaggtgaagaagaggaaggagaatggggtcgggatagtcccttgtctctctacactgaacctccaggggtctatgactggcctccctgggctcactgtcctcttccagtggggccaggtcttgcttggatgagccctaatcagttgtatgagcctttcaatcagtcttcctatgtgcaggcaacctgttgtgtacctcctgtggcaatgccagtgtcagtgccaggaagaacacctggagactctgtgtctcagctagctcgaccttcgcacctacccctgcccatgggcccttgctataaccttcaatcacaggcctcccagagtgggagggccaagcctcgggatgtactgctgcctgtagatgagcccagttgctcttccatttctggagccaactctcagtctcaagccaagcctgtaggtatcacccatggtatccctcagctgcccagggtgcggcctgaacccttccagcttcaacccaatcactatcgagcttccaaccttgacctgtcaaaggagaggggtgagcaaggtgcctctctctctaccagctactcctctactgccatgaatggaaacctagccaagtagttatcaattccagaatggagacctgagcatgtgaattgggggtcaagggttgggcagaggggtaggggtgagtagtgctgttccacttgaaaatcttattggctaagataaactcctctgagttttcactttgcccaccttcctctgccatctttggccaggttcagctcaattatatctgcctagggaggctgctgcttctgtgctccaatttttgctttggagtttcttgtgacctatacagggcttgggatgcatccttattgcaggctgaacccctcttctctggtcccttgctgactaatgaaccactgatgagcagccaattttttgcactgactctcacccagagatgggaggcttgctagctattagcaatataaagttatggtacatagccttcctaaccccattcctttctagcacatcaaatgactataacctatttatcccagttagttatgtttagagtgggtcaagtagcatgtgtctcgctccctgttgctgtggactttggtatcacaactagatgatgtccttttaacctggcctgccatagtctagattgaagacaaaaacactaatcctcataagggggtgattaaatctgtccatattcaagcagaatacaaagatagatagcttatggaaaatgctgtgaggtcttgagctgtggaataataatatatctatcttgtttgtttcctaagcctgtggctgttgcaggcattttagaacttcccccttttcctgtccctgaaaaatttcatctcatcacaatgaatgtagtatcttaactgagttaaaggaagagaagccacctaaattgggcttgactatatagagcattgggtgttgtgagatgcaaaagaggaaaacagacagggtgatccatttatctaaacaccaagaagggatctcagactttggaacttgaagcaactcagattaggagggaaaacttttcccatcctttctcccaaaccaaaagtaaataaataaataaacccttggccgcccctttgtaaagtggttccaagcttgtctaagacaggatgccaaaggcatctcacctgttgtaggcacactaaaaagcttttatcttccagttagccaagttcttagcattttcaaaggaccaggcttcttgagaggaaagattgtaatgtacccttgctctgccaaactacagggctgatttgcatggcagttgcagtcctttctgaaatgaacagcaccctctcttctcttgcatccatgcaggtttctttttcttttttctatattttttttctggacaaggtcccacactgtagttctctctagcctggaacttaccatgtagaccacgatggcttctaactaacagagatccacctgcctctgcctccaagcaagcccagcctaaatgttggaaacactagggaagagcttggtgtcaggaaaacattgcaacaggctcatcttgaccaggttcaacatttccttaatcatctatatgaaacagtttctttaaaattctccatcaggcccggtgtggaggtatgtgccttttatctcaacactcagaaggaagaggcaattggagttatatgagtgtgagaccagcctgttctacatagtgagttctaggccactagggctacatagcaagatagaccttgttttaaatttttttctgtattggaatatgaaatattttggggatcgatatgttgatcaaagttagaactttttaaatcttctgcccatgtccctcttctcacctcaaagccatcttcacatctactaagtgaagatatcaatagggctcttgctcaaaataaggcaatttaccttgagaacagatgctggtggccttctgagtcaagattgctttgtagacatactttctttgccagggcatatattcataatatttttcacagtttctaagcttgggattggccctgtgcctttcccccaaaagagcagatttgattggtaaaatctaaccccttgtaagtaacttcataactttgcatggcaccagcactagaaccacccctcaaggacctcagatcagctctcttggatgactttggctacatcacgaagaagtgcatctcttgtggatctggataatgaaagtgaaaaaggccttccttaccccaaccacatcgtcataagagacacatgcccagattttaaccacttggcctcctaccaaagctgttctgggaaggtgaggctgaacttagcctggttgggtggcaggaagtaagcactcaatgcctttacaacaggaaaagccacacctcttgcctagtcaaggtagcttgagttgctgtttacatgtcgctgccatctacagcaggcactagagctgtccacagcctccatagcccactggagaagggctaccagcctgtttggaagaggaagaaggaatctaccctcacagttcaggaggacagcacagtgcaatatctctgaagggacctgctataacttggatatattttgctatgctttgaaacttcataccttagagtaggacctgaggcaaagtagaagtttgtggattgggtaacatcattgccttccaagtcctttcactgatctggctgctagcctagggacaaaatatttacaacctaaaatgggatagccaagatgtctacagaagcagactccttttgagtatctaaagcaaatactaactgcagcctagaaggggaaagaagatttggctgcggatatatggtaatggcaacatgattaccaatgggatatgtaaccaatctctaggtatctgtccaaacatggacaagtacttcctttgctggcaccaaatatgctacaaactaccagaagtgctgctattgtgttcaccctaaggctgtggccagatgaacctcctgactccattgcaggtccttggacaatgcatttggatgtgactttaagggaagatgtgctgacagaaggaggctagctttatgttgttacccagtactactatatagtgtcagctctgatcctgagaatttcttagtaacccagtccatagaggaccagtgactgtgggtatggaaagccacctagcaggcctcttccaaagccaggatgccaatttcttggtgctttagattctccaaggaagctctgccttggggatcatctggctttagcttagataccagaccacaagcctcgtggataatggtttcagtaaatgccagtgtccctcctcttagtgctatttcaaggagggaagtagctttccacactcagaatgttgggtgcatgatttcatcctgccaccagtggctttttcacctgcaccagtgccaccactgccttcagcccttaggctcacttgctagagggaggcatgggggtttaccagggatacttcaagtttacccatgtgtatgttgcatccaagttgtttttaatatatatatataaatatatatatcaaaagctaattatctatttttatattgtttgcattttatattcatggaaacaatgtttatcaattgttcttttctgatatattttatttttaagcggtgctgtttacagttttaaacaaaaaaaaaagatgacacaaaaaatgctgctgctcgtgcagagggatgtcatctcaaagtgtctaggaccctccctcttttccctctcccacctggctgtgtcacactattcattctctttccacacggtaaggacagaagatgcccatgtaccgaagaccaataccaggtaacgcaagcatgtggaccctggcagacagcatgttttctagctttcattttatttaccttttgagcccttccttctcttgtgtagacgttttgaaagtactcctgcctcttgacttttattcttttcctttctttatattttgtgcttgttttttgcaaagtatcatgcccatattgtgtggtttgttgtattaagtttctccaatggagcattaggggctcattcaacaccaggagactcgggatgacagatgacaatgctatttcaacctccaggaattggtcatctccttgggagacacccttgaatgagacttgccagcaatttagaagatggcaacattgtccagaaaaggcattaggtgattccttggtccctcttgctactttgggagaatctctccacatcctgcctcctccttcccttccctgttccagctcacaagtggatggtacttttggaaactgaactcttgttgttttgtttttttattgtaagtcttagcccttaatggctgagttgtctctttaaccaggatggcacttttaggaatggtgactgttacagtcatggctgggtggcctgatccacggtttctccatcctgtagtcattccaaagaagaaaatccacatggcatgtacatttgtgagggttgggtctattaagagggggctttaattattcctgttactattggtgcttattttccccgctgtatatgggggtggggaagggctggggtaaaccagaaatgtatatttaggtcacgttaaataaaatggctgggggaagaagaaagatatgagtctaggtgtaagagttctctcttccaggacagaaaggggattgtagcttgttgctgcacctgagcttcccttccgcattcctgagacatcatggaatcatctgcatttgttacctctccatccctagcactcactggcatctggatatcctgatacactgcctgtatgttacaacatatatttcatttgctgaatgctaataaacttctgtgctcatgagggacctccagtca

SEQ ID NO: 26 - protein, 1132 aa, Mus musculus
MESQQDEAVQTKGASTSSDAQDQGAEKGAKNKTTEATEGPTSEPPLSGPGRLKKTAMKLFGGKKGICTLPSFFGGGRSKGSGKVSSKKSLNKSKTHDGLSEASQGPEDVVIEETDLSTPLSKSSAQFPSSQSANGALEIGSKHKTSGTEAIEKAGVEKVPSVHKPKKSLKSFFSSIRRHRKGKTSGADQSVPGAKELEGARTRSHEHVSSISLPSSEEIFRDTRKENAKPQDAPGPKMSPAQVHFSPTTEKAACKNPEKLTRTCASEFMQPKPVLEGGSLEEPHTSETEGKVVAGEVNPPNGPVGDQLSLLFGDVTSLKSFDSLTGCGDIIAEQDMDSMTDSMASGGQRANRDGTKRSSCLVTYQGGGEEMALPDDDDNDDEEEEEEEEEEEEEEEEEEEEEEEEEEEELLEDEEEVKDGEENDDLEYLWASAQIYPRFNMNLGYHTAISPSHQGYMLLDPVQSYPNLGLGELLTPQSDQQESAPNSDEGYYDSTTPGFEDDSGEALGLAHRDCLPRDSYSGDALYEFYEPDDSLEHSPPGDDCLYDLRGRNSEMLDPFLNLEPFSSRPPGAMETEEERLVTIQKQLLYWELRREQREAQEACAREAHAREAYARDTHTRESYGRNVRARETQALEAHSQEGRVQETKVRQEKPALEYQMRPLGPSVMGLVAGTSGGSQTSHRGTTSAFPATSSSEPDWRDFRPLEKRFEGTCSKKDQSTCLMQLFQSDAMFEPDMQEANFGGSPRKAYPSYSPPEEPEEEEEEKEGNATVSFSQALVEFTSNGNLFTSMSYSSDSDSSFTQNLPELPPMVTFDIADVERDGEGKCEENPEFNNDEDLTASLEAFELGYYHKHAFNSYHSRFYQGLPWGVSSLPRYLGLPGVHPRPPPAAMALNRRSRSLDNAESLELELSSSHLAQGYMESDELQAHQEDSDEEGEEEEGEWGRDSPLSLYTEPPGVYDWPPWAHCPLPVGPGLAWMSPNQLYEPFNQSSYVQATCCVPPVAMPVSVPGRTPGDSVSQLARPSHLPLPMGPCYNLQSQASQSGRAKPRDVLLPVDEPSCSSISGANSQSQAKPVGITHGIPQLPRVRPEPFQLQPNHYRASNLDLSKERGEQGASLSTSYSSTAMNGNLAK

SEQ ID NO: 27 - DNA, 2571 bp, Mus musculus
atggagagtcaacaggatgaagctgttcaaaccaaaggagcatcaacttctagcgatgcccaagaccagggagctgaaaagggagccaagaataagacaactgaggcaacagaaggaccaacatcagagcctcccttatcaggcccaggttgtggtgacattatagcagaacaggacatggacagcatgacagatagcatggcctctggtggccagagggccaaccgagatgggaccaagcgaagttcctgcttggtaacctaccaaggaggtggggaggagatggccttgccagatgatgatgacaatgatgatgaagaagaagaagaagaagaagaagaagaagaagaagaagaagaagaagaagaagaagaagaagaagaagaagaagaagaagaattattagaggatgaagaggaggttaaggatggagaggaaaatgatgacttagagtatctgtgggcaagtgcccagatatacccaagatttaatatgaatctgggctaccatactgctatatctcccagccaccagggctacatgcttcttgacccagttcagtcttatcctaatctaggccttggagaacttttgacacctcagagcgaccagcaagagtctgctcccaatagtgatgaaggttattatgactctaccacaccaggatttgaggatgattcaggtgaggctctgggacttgcccacagggattgtctacccagggacagctatagtggtgatgcgctatatgagttctatgagccagatgacagtcttgagcactccccacctggggatgactgcctatatgacctccgtggacgcaactcagagatgttggatcctttcttgaacttggagcccttttcttctcggccacctggggcaatggaaaccgaggaagaacggctagtgaccatccagaagcagttgctatactgggaacttcgccgggagcagcgtgaggctcaagaggcatgtgccagagaggctcatgccagagaagcttatgcccgagacacccatactcgggaatcttatggcagaaatgtccgagcccgagaaactcaggccttagaggctcattctcaagagggtcgagtccaagagaccaaggtacggcaggagaagcctgccttggagtatcagatgaggcccttaggtccctcagtgatgggtctggtggcagggacatcaggaggttctcagacttctcaccggggaaccacttcagctttccctgctacttctagcagtgagccagactggagggatttccgtcctctggagaagcgttttgaggggacttgttccaaaaaagatcaaagtacttgtttaatgcagctcttccagagtgatgccatgtttgagccagacatgcaagaagcaaattttggaggctcacccaggaaggcgtaccctagttactcaccccctgaggagccagaggaagaagaagaggagaaggaagggaatgccactgtgagtttctcacaggccctagtagagttcaccagcaatggaaacctttttaccagcatgtcctacagctctgattctgactcatccttcactcaaaacctccctgagctgccccccatggtcacctttgatattgctgacgtggaacgagatggagaaggcaagtgtgaagagaatcctgagttcaacaatgacgaagatcttacagcctccttggaagcttttgagctgggttattaccacaaacatgccttcaacagctatcacagtcggttctaccaaggcctgccctggggagttagcagcctccctcgatacttaggactccccggagtccaccctcgacctcctcctgctgcaatggcactcaataggagaagccgttcccttgacaatgcagagtctctggagctggagctctccagttctcacctagcccagggttacatggagtctgatgagctccaggcccaccaggaagattcagatgaagaaggtgaagaagaggaaggagaatggggtcgggatagtcccttgtctctctacactgaacctccaggggtctatgactggcctccctgggctcactgtcctcttccagtggggccaggtcttgcttggatgagccctaatcagttgtatgagcctttcaatcagtcttcctatgtgcaggcaacctgttgtgtacctcctgtggcaatgccagtgtcagtgccaggaagaacacctggagactctgtgtctcagctagctcgaccttcgcacctacccctgcccatgggcccttgctataaccttcaatcacaggcctcccagagtgggagggccaagcctcgggatgtactgctgcctgtagatgagcccagttgctcttccatttctggagccaactctcagtctcaagccaagcctgtaggtatcacccatggtatccctcagctgcccagggtgcggcctgaacccttccagcttcaacccaatcactatcgagcttccaaccttgacctgtcaaaggagaggggtgagcaaggtgcctctctctctaccagctactcctctactgccatgaatggaaacctagccaagtag

SEQ ID NO: 28 - protein, 856 aa, Mus musculus
MESQQDEAVQTKGASTSSDAQDQGAEKGAKNKTTEATEGPTSEPPLSGPGCGDIIAEQDMDSMTDSMASGGQRANRDGTKRSSCLVTYQGGGEEMALPDDDDNDDEEEEEEEEEEEEEEEEEEEEEEEEEEEELLEDEEEVKDGEENDDLEYLWASAQIYPRFNMNLGYHTAISPSHQGYMLLDPVQSYPNLGLGELLTPQSDQQESAPNSDEGYYDSTTPGFEDDSGEALGLAHRDCLPRDSYSGDALYEFYEPDDSLEHSPPGDDCLYDLRGRNSEMLDPFLNLEPFSSRPPGAMETEEERLVTIQKQLLYWELRREQREAQEACAREAHAREAYARDTHTRESYGRNVRARETQALEAHSQEGRVQETKVRQEKPALEYQMRPLGPSVMGLVAGTSGGSQTSHRGTTSAFPATSSSEPDWRDFRPLEKRFEGTCSKKDQSTCLMQLFQSDAMFEPDMQEANFGGSPRKAYPSYSPPEEPEEEEEEKEGNATVSFSQALVEFTSNGNLFTSMSYSSDSDSSFTQNLPELPPMVTFDIADVERDGEGKCEENPEFNNDEDLTASLEAFELGYYHKHAFNSYHSRFYQGLPWGVSSLPRYLGLPGVHPRPPPAAMALNRRSRSLDNAESLELELSSSHLAQGYMESDELQAHQEDSDEEGEEEEGEWGRDSPLSLYTEPPGVYDWPPWAHCPLPVGPGLAWMSPNQLYEPFNQSSYVQATCCVPPVAMPVSVPGRTPGDSVSQLARPSHLPLPMGPCYNLQSQASQSGRAKPRDVLLPVDEPSCSSISGANSQSQAKPVGITHGIPQLPRVRPEPFQLQPNHYRASNLDLSKERGEQGASLSTSYSSTAMNGNLAK

SEQ ID NO: 29 - DNA, 20 bp, Artificial
  sense
gagccaagaacaaggcagtt

SEQ ID NO: 30 - DNA, 20 bp, Artificial
  antisense
ttcucgguucuuguuccguc

SEQ ID NO: 31 - DNA, 21 bp, Artificial
  Sense strand
ggccuaaaaggcuuuuuuatt

SEQ ID NO: 32 - DNA, 21 bp, Artificial
  antisense
ttccggauuuuccgaaaaaau

SEQ ID NO: 33 - DNA, 3306 bp, Pan troglodytes
  misc_feature (2420)..(2420): n is a, c, g, or t
atggagacccaaaaggatgaagctgctcaggccaagggagctgcaacctctgggagtacccgtgaacaaacagcagaaaaaggagccaagaacaaggcagctgaggcgacagaaggaccaacctcagagccatcctcgtccggcccaggtaggctgaagaaaactgccatgaaactctttggtggcaagaagggtatctgtactctgcctagtttctttggagggggacggagcaaaggttctgggaaaggcagctccaagaaaggtctcagcaagagcaagacccacgatggcctgagtgaagcagcccatggccctgaagatgttgtcagtgaaggaactggcttctccctgcctttgcctgagttaccctgccaatttcccagctctcagagtgcccatggggctttggagacaggctccagatgtaagacatctgtggctggagccacagagaaagctgtggctgagaaggttccctctatgcccaagccaaagaaaggcctaaaaggcttttttagcagtatccgccgtcaccggaagagcaaggtcactggggctgagcaaagtgagccaggggccaaggggcctgagagggtcagagccaggcctcatgagcacatgagctcagcccctcaggtgccctgctttgaggagaccttccaagcccctagaaaggaaaatgctaacccccaagatgcccctgggccaaaagtttctccaacaccagagccttctccaccagctactgagaaaatggcctgtaaagatccagaaaaacccatggaggcctgtgcctcagcacatgtgcaacccaagcctgcccctgaagccagtagcctagaggagccccatagcccagaaacaggggagaaggtagtagcaggagaggtaaacccacccaatggccctgtgggggacccactgagcctcttgtttggggatgtgacatccctgaaaagctttgattcattgacaggttgtggtgacataatagcagaacaggacatggacagtatgacagacagcatggcctctgggggccagagagcaaaccgagatgggaccaagcgaagttcctgcctggtgacctaccaaggaggtggggaggagatggccttgccagatgatgatgacgaggaggaagaagaagaggaagaagaggtggaattagaggaggaagaagaggaggttaaggaggaggaagaagatgatgacttagaatatctgtgggaaactgcccaaatgtatccacggcccaatatgaacctgggctaccatcccaccacatccccaggccaccacggccacatgctccttgacccagttaggtcttatcctggcctagcccctggagaacttttgactcctcagagtgaccagcaagaatccgcccccaatagtgatgaaggttattatgactccaccacacctggatttgaggatgattcaggtgaggccctggggcttgtccgcagggattgtctaccccgagacagctacagtggagatgccctatatgagttctatgagccagatgacagccttgagaactctccacctggagatgactgcctttatgacctccatggtcgaagctctgagatgtttgtccccttcttaaactttgagcccttttcttcctcccggccacctggggcaatggagacagaggaagaacggctagtgaccatccagaaacagttgttgtattgggagcttcggcgggagcagcttgaggcccaggaggcacgtgcccgagaagctcatgccagggaggcccacgccagggaggcctatactcgagaggcttatggcagggaagcctatgccagggagacccacacttgggaagctcatggcagggaggccagaacccgagaagcccaggcccgagaggttcgttctagagagactcaagtccgagagacccaggcccggcaggagaagcccgtcttagagtatcagatgaggcccttaggcccatcagtgatgggcctggcagcaggggtatcagggacctctcagatttcccaccggggaattacctcagctttccccaccactgcgagcagcgagccagactggagggacttccgtcctctggagaagcgttatgaaggaacctgctccaagaaagatcaaagtacctgcctgatgcagctcttccagagtgatgccatgtttgagccagacatgcaagaagcaaattttggaggatctcccaggagggcctaccccacttattcaccccctgaagatccagaggaagaggaggttgagaaggaagggaatgccactgtgagtttctcacaggccctggtagagttcaccagcaatgggaacctcttttccagcaagtgtgaagagaatcctgagttccacaatgatgaagatcttgcagcctccttggaagcctntgagctgggctactatcacaaacatgccttcaacaactaccatagtcgattctaccaaggcctgccctggggtgtgagcagcctccctcgatacttgggactgcctggcctgcacccccgacctccacctgctgctatggccctcaacaggagaagccgctctctcgacactgcagagaccctggagatggagctctccaattcccacttggtccagggctacctcgagtctgatgagctgcaggcccagcaggaagattcagatgaggaggacgaagaggaggaagaaggagaatggagccgagacagtcccctttccctctatactgaacccccaggggcctatgattggcctgcttgggctccctgtcctcttccagtggggccaggtcctgcctggataagccccaaccagttggacaggccttccagccagtctccatataggcaggcaacctgttgcatacctcctatgaccacgtcaatatcactatcagtgccagagtcaagggcacctggggaatctgggcctcagctagctcgtccctcacacctacacctgcccatgggcccttgctatattctccagccacaggcctcccagagcatgagggccaggcctcgagatgtgctgctgcctgttgatgagcccagttgctcttccagttctggaggcttcagccccagccctctaccacaggccaagcctgtgggcatcacccatggcattcctcagctgcccagggtccggcctgagcacccccagcctcagcccactcactatgggccttccagccttgacctgtcaaaggagagggctgagcaaggtgcctctcttgccaccagctactcctccactgccatgaatggaaacctagccaagtag

SEQ ID NO: 34 - protein, 1101 aa, Pan troglodytes
  misc_feature (807)..(807): Xaa can be any naturally occurring amino acid
METQKDEAAQAKGAATSGSTREQTAEKGAKNKAAEATEGPTSEPSSSGPGRLKKTAMKLFGGKKGICTLPSFFGGGRSKGSGKGSSKKGLSKSKTHDGLSEAAHGPEDVVSEGTGFSLPLPELPCQFPSSQSAHGALETGSRCKTSVAGATEKAVAEKVPSMPKPKKGLKGFFSSIRRHRKSKVTGAEQSEPGAKGPERVRARPHEHMSSAPQVPCFEETFQAPRKENANPQDAPGPKVSPTPEPSPPATEKMACKDPEKPMEACASAHVQPKPAPEASSLEEPHSPETGEKVVAGEVNPPNGPVGDPLSLLFGDVTSLKSFDSLTGCGDIIAEQDMDSMTDSMASGGQRANRDGTKRSSCLVTYQGGGEEMALPDDDDEEEEEEEEEVELEEEEEEVKEEEEDDDLEYLWETAQMYPRPNMNLGYHPTTSPGHHGHMLLDPVRSYPGLAPGELLTPQSDQQESAPNSDEGYYDSTTPGFEDDSGEALGLVRRDCLPRDSYSGDALYEFYEPDDSLENSPPGDDCLYDLHGRSSEMFVPFLNFEPFSSSRPPGAMETEEERLVTIQKQLLYWELRREQLEAQEARAREAHAREAHAREAYTREAYGREAYARETHTWEAHGREARTREAQAREVRSRETQVRETQARQEKPVLEYQMRPLGPSVMGLAAGVSGTSQISHRGITSAFPTTASSEPDWRDFRPLEKRYEGTCSKKDQSTCLMQLFQSDAMFEPDMQEANFGGSPRRAYPTYSPPEDPEEEEVEKEGNATVSFSQALVEFTSNGNLFSSKCEENPEFHNDEDLAASLEAXELGYYHKHAFNNYHSRFYQGLPWGVSSLPRYLGLPGLHPRPPPAAMALNRRSRSLDTAETLEMELSNSHLVQGYLESDELQAQQEDSDEEDEEEEEGEWSRDSPLSLYTEPPGAYDWPAWAPCPLPVGPGPAWISPNQLDRPSSQSPYRQATCCIPPMTTSISLSVPESRAPGESGPQLARPSHLHLPMGPCYILQPQASQSMRARPRDVLLPVDEPSCSSSSGGFSPSPLPQAKPVGITHGIPQLPRVRPEHPQPQPTHYGPSSLDLSKERAEQGASLATSYSSTAMNGNLAK

SEQ ID NO: 35 - DNA, 3393 bp, Canis familiaris
atggagactggaaaggatgaagcttctcaggccaaaggagcatcagcctctatggacactcaggaccaaggggcagagaaaggagctaagaacaaggcagctgagataacagaagggccagtgtcagaaccaccctcatctggcccaggtaggctaaggaaaactgccatgaaactttttggtggcaagaagggcatctgtaccctgcctagtttctttggaggggggcgaagcaaaggttctgggaaaagcatctccaggaagggtcttagcaagagcaagactcatgatggcctgactgaagcagcccatggccctaaagacatttttagtgaaggaaccagctttgccttacctttgcctgaatcatcctgccaacttcccagttcccagagtgcccatggggttttggagacagactccagatgcaagacatctgtggctggagccacagagaaagctgaggctgagaaggctctttctctgtccaagccaaagaaaggcctgaaaggtttttttagcagtatccgccgtcaccggaaaagcaaggtctctggggctgaaaaaagtgagttaagggccaaggggcctgaaggggctagagctaggcctcatgaatatgtgagctcagtcattctgcaccatactgaggatatcctccaagcccccagaaaggaaaatgccaaacctcaagatgctcctgggtcaaaactttctttagtaccagagccttttccagctgtcattgagaagacagcctgtaaagatccagaaaaatctgtggaggcctgtgcttcagcacttatgcaacccaagcctaccttagaagctagcggcccagaagagccccatagtccagaaacaggggagaaagtggtgtcagaagaggtaaatccaccaaatggccctgttggggaccagctgagcctcttgtttggggatgtcacatccctgaaaagttttgactcactgacaggttgtggtgatatcatagcagagcaagacatggacagtatgacagacagcatggcttctggaggccagagggccaatcgagatgggaccaagcgaagttcctgcctggtgacctaccaaggaggtggagaggagatggccttgccagatgatgaggaggaggaagaggaggaagatgaggtggaattagaggaggaagaagaggatgtcaaggaggaagaagattatgacttagaatatctctgggcaagtgcccagatgtatccgaagtctaatctgaatttgagctaccatcccatcacatccccaggccaccatggctacatgctccttgactcagttcgatcttatcctggcttagctcctggggaacttttgactcctcagagtgaccagcaagagtctgcccccaatagtgatgagggttattatgactccacaacacctggatttgaggatgattcaggtgaggccttggggcttatccgcagggattgccttccccgagacagttacagtggagatgccctgtatgagttctatgagccagatgatagtcttgagacctccccacctgtggatgactgtctttatgacctccatggtcacagctctgagatatttgaccccttcttgaactttgagctcttttcttcctctaggccacctggggcaatggagacagaggaagaacggctagtgactatccagaaacagttgttgtgttgggagcttcggcgagagcagcttgaggcccgagaggcccaggaggcatgtgcccaagagattcacaccagggaggcctatacccaagaaactcatgccagagagacccatgtccgagaagctcatgtcagagaggcccaagcccgagaggcctatgccagggaagcccaggtccgggaggcccatgctcaagaggctcaagtccgagaggttcaggcctggcaggagaagcccatcatagaatatcatatgaggcccttaggcccatcatcagtgatgggcctggtagcaggggcatcaggggcctctcagacatctcatcgggcaaccacctcagctttccctgccactgcaagcagtgagccagactggagggacttccgtcctctggagaagcgttttgagggaatctgctccaagaaagatcaaagtacctgtctgatgcagctcttccaaagtgatgctatgtttgagccagacatgcaagaagcaaactttggaggatctcccaggagggcctaccctacttattcaccctctgaggagccagaggaagaggaggctgaaaaggaagggaatgccacagtgagcttctcacaggcccttgtggagtttaccagcagcgggaacctcttctccagcatgtcctgcagctctgattctgactcatccttcgctcaaaaccttcctgaactgccccctatggtgacttttgacatcgctgatgtggaacgggagggggaaggcaagtgtgaagagcatcctgagttccacaatgaagaccttgcagcctccttggaagcttttgagttgggctactaccagaaacatacattcaacaactaccacagccgattctaccaaggtcttccctggggtgtgagcagtctccctcgctatttgggactgcctggcatgcaccctcgacctccacctgctgctatggccctcaacaggaggagtcgctccctggacactgcagagaccttggagctggagctttccaattcccgccctgcccagggatacatagtgtctgatgagcttcaggctcagcaggaagattcaaatgaagaagaagaagaggaagaatggggtcgagacagtcctttgtccctttatactgaaccaccaggggcctacgactggtctgcctgggctccctgtcctctcccagtggggccaggtcctgcctggataagtcccagccagttggatgggtcttctagccagtctccatatgagcagacagcctgttgcatacctcctgtggccatgtcaatgttgttgtcagtaccagggccagagccaagggtacctgggatatccaggcctcagctatctcggccttcgtacctacccctgcctatggtctcttgttataacctgcagccacaggcctttcagagtgtgagggccaggcctcaagatatgctgctgcctattgatgagcccaattgcttttccagttctggaggcttcagctccagtcctctgtcccaggccaagcctgtgggcatcactcatggcatccctcagctgcccagggtccagcctgagccccaacaacctcaacccattcactgtaaggtttccagccttgacctatcaaaggagatggctgagcaaggtgcctctctcccccttcccaccagctactccactgctgtgaatggaaagctagctgagtag

SEQ ID NO: 36 - protein, 1130 aa, Canis familiaris
METGKDEASQAKGASASMDTQDQGAEKGAKNKAAEITEGPVSEPPSSGPGRLRKTAMKLFGGKKGICTLPSFFGGGRSKGSGKSISRKGLSKSKTHDGLTEAAHGPKDIFSEGTSFALPLPESSCQLPSSQSAHGVLETDSRCKTSVAGATEKAEAEKALSLSKPKKGLKGFFSSIRRHRKSKVSGAEKSELRAKGPEGARARPHEYVSSVILHHTEDILQAPRKENAKPQDAPGSKLSLVPEPFPAVIEKTACKDPEKSVEACASALMQPKPTLEASGPEEPHSPETGEKVVSEEVNPPNGPVGDQLSLLFGDVTSLKSFDSLTGCGDIIAEQDMDSMTDSMASGGQRANRDGTKRSSCLVTYQGGGEEMALPDDEEEEEEEDEVELEEEEEDVKEEEDYDLEYLWASAQMYPKSNLNLSYHPITSPGHHGYMLLDSVRSYPGLAPGELLTPQSDQQESAPNSDEGYYDSTTPGFEDDSGEALGLIRRDCLPRDSYSGDALYEFYEPDDSLETSPPVDDCLYDLHGHSSEIFDPFLNFELFSSSRPPGAMETEEERLVTIQKQLLCWELRREQLEAREAQEACAQEIHTREAYTQETHARETHVREAHVREAQAREAYAREAQVREAHAQEAQVREVQAWQEKPIIEYHMRPLGPSSVMGLVAGASGASQTSHRATTSAFPATASSEPDWRDFRPLEKRFEGICSKKDQSTCLMQLFQSDAMFEPDMQEANFGGSPRRAYPTYSPSEEPEEEEAEKEGNATVSFSQALVEFTSSGNLFSSMSCSSDSDSSFAQNLPELPPMVTFDIADVEREGEGKCEEHPEFHNEDLAASLEAFELGYYQKHTFNNYHSRFYQGLPWGVSSLPRYLGLPGMHPRPPPAAMALNRRSRSLDTAETLELELSNSRPAQGYIVSDELQAQQEDSNEEEEEEEWGRDSPLSLYTEPPGAYDWSAWAPCPLPVGPGPAWISPSQLDGSSSQSPYEQTACCIPPVAMSMLLSVPGPEPRVPGISRPQLSRPSYLPLPMVSCYNLQPQAFQSVRARPQDMLLPIDEPNCFSSSGGFSSSPLSQAKPVGITHGIPQLPRVQPEPQQPQPIHCKVSSLDLSKEMAEQGASLPLPTSYSTAVNGKLAE

SEQ ID NO: 37 - DNA, 3360 bp, Bos taurus
atggagacccgaaaggatgaagctgctcaggccaagggaacaacagtctctatggatacccaggatcaagggacagagaaaggggccaaaaacaaggcatctgagacaacagaaaggcccacatcagagccaccctcatctggtccaggtaggctgaagaaaactgccatgaaactctttggtggcaagaagggcatctgtaccctgcctagtttctttggagggggacgaagcaaaggttctgggaaaggcagttctaagaagggtcttagcaagagcaagacccacgatggcctgagtgaagcagtccatgaccctgaagacattgtcagtgaaggaactggcctctccttacctttgcctgagtcatcatgccaacttcccagctctcagagtgtccatgggtctttggagacagactccagatgcaagaggtctgtggctggagccacagagaaagctggggctgagaaggctcactttgtgcccaagccaaaaaaaggcctgaaaggttttttcagtagtatccggcgtcaccggaagagcaaggtctctggggctgagcaaagcaatccaggagccaaggagtctgagggggccagagcaaggcctcatgagtatgtgagctcagcccttctgtcccacgctgatgaggtcctccaagccccaagaaaggaaaatgccaaatttcaagatgtccctgggccgaacattccttcaatatcagagacttctctggcagccactgagaaagcagcctttaaagatgcagaaaaaaccttggaggcctttgtctcagcattcttgcagcccaaacctgcccctgaagccagtggcccagaggagttccatagtccagaaacaggggagaagcttgtggcaggagaggtaaatccacccaatggccctttaggggaccagctgagcctcctctttggggatgtcacatctctgaagagttttgactcactgacaggttgtggtgacataatagcagaacaggatatggatagtatgacagatagcatggcctctggaggccagagggccaaccgagatggaaccaaacgaagttcctgcttggtgacctaccaaggagggggcgaagagatggccttgcctgatgatgatgatgaggaagaagaggaagaggaggtggaattagaagagggagaagaagtcaaggaggaagaagatgatgatttagagtatctgtgggcaagttcccagatgtacccaaggcccatactaaatccaggctaccatcccacaacatccccaggccatcttggctacatgcttcttgacccagttaggtcttatcctggctcagcccctggggaacttttgactcctcagagtgatcagcaagagtccgcccccaatagtgatgagggttattatgactccactacaccaggacttgaggatgattcaggtgaggccctggggcttgtccatagggattgcttgccccgagacagctatagtggtgatgccctctatgagttctatgagccagatgatagtcttgagaactccccacctggggatgactgcctttatgacctccattgtcacagctctgagatgtttgaccccttcttgaactttgagcccttttcttcccctcggccacctggggcaatggagacagaggaagaacggctagtagccatccagaaacagttgctatattgggagcttcagcgggagcagcgagaggcccgggaagcatgtgcccaagaggctcacaccagagaggcccatgctcgagaggcccacacccgagaagcttatgtcagagaggcccgacctcgagaggcctacgccagggaggcctgtggccaagaggtctgtggtcgagagcttcaagtccgagaggcccaggtgcggcaggagaagcccgtcatagagtatcaaatgaggcctttaggcccatcagtgatgggtctggtggaaggggcatcaggggcctctcagacttcccacagaggaaccacatcagctttccctgccactgcaagcagcgagccagactggagggacttccgtcctttggagaagcgttttgagggaacctgctccaagaaagatcaaagtacttgcctgatgcaactcttccagagtgatgctatgtttgagccagacatgcaagaagcaaattttggaggatctcccaggagggcctaccctacttattcaccccctgaggagccagaggaagaggatgttgagaaggaagggaatgctacagtgagtttctctcaggccctcgtggagttcaccagcaatgggaacctcttctccagcatgtcctgcagctctgactctgattcgtcctttactcaaaaccttcctgagctgcctcccatggtgacctttgatattgctgatgtggaacgggatggggaaggcaagtgtgaagaaaaccctgagttccacaatgatgaagaccttgcagcctccttggaagcttttgaactgggctactatcagaaacatgccttcaacaactaccgcagccgattctaccaaggcctaccctggggtgtgagcagtctccctcgatacttgggactacctggcatgcaccctcgtcctccacctgctgccatggccctcagcaggaggagccgctcccttgacactgctgagaccctggagctggagctctccaattctcacctgacccaaggctatatggagtctgatgagcttcaggcccagcaagaagattcagaagaagaagaggaggaggaagaatggggccgagacagtcccctatctctctatactgaacctccagtaacctatgactggcctgcctgggctccctgtcctctcccagtggggcctggccatgcctggataagtcctagccagttggatgggccttctagccatccatttgggcaggcagtctgttgcatatctcctttcaccatgtcaatgttgctctcagtatcagggccagagccaagagcacctggggaatccaagtctcagctagctcggccttcgcacctacccttgcccatgggcccttgttataacctccagccaaaggcctcccagagtgtgagggccaggcctcaagatgtgctgctgcctgttgatgagcccagttgttcctccatttctggaggcttcagccctagccctgtgccccaggccaagcctgtgggcattactcatggcatccctcagctgcccaggatccggcctgagcccccagagcctcagcccattcactatggggcttcccgccttgacctgtcaaaggagaaggctgagcaaggtgcctctctccccaccagctgctcctccactgctatgaatggaaaactagctgagtag

SEQ ID NO: 38 - protein, 1119 aa, Bos taurus
METRKDEAAQAKGTTVSMDTQDQGTEKGAKNKASETTERPTSEPPSSGPGRLKKTAMKLFGGKKGICTLPSFFGGGRSKGSGKGSSKKGLSKSKTHDGLSEAVHDPEDIVSEGTGLSLPLPESSCQLPSSQSVHGSLETDSRCKRSVAGATEKAGAEKAHFVPKPKKGLKGFFSSIRRHRKSKVSGAEQSNPGAKESEGARARPHEYVSSALLSHADEVLQAPRKENAKFQDVPGPNIPSISETSLAATEKAAFKDAEKTLEAFVSAFLQPKPAPEASGPEEFHSPETGEKLVAGEVNPPNGPLGDQLSLLFGDVTSLKSFDSLTGCGDIIAEQDMDSMTDSMASGGQRANRDGTKRSSCLVTYQGGGEEMALPDDDDEEEEEEEVELEEGEEVKEEEDDDLEYLWASSQMYPRPILNPGYHPTTSPGHLGYMLLDPVRSYPGSAPGELLTPQSDQQESAPNSDEGYYDSTTPGLEDDSGEALGLVHRDCLPRDSYSGDALYEFYEPDDSLENSPPGDDCLYDLHCHSSEMFDPFLNFEPFSSPRPPGAMETEEERLVAIQKQLLYWELQREQREAREACAQEAHTREAHAREAHTREAYVREARPREAYAREACGQEVCGRELQVREAQVRQEKPVIEYQMRPLGPSVMGLVEGASGASQTSHRGTTSAFPATASSEPDWRDFRPLEKRFEGTCSKKDQSTCLMQLFQSDAMFEPDMQEANFGGSPRRAYPTYSPPEEPEEEDVEKEGNATVSFSQALVEFTSNGNLFSSMSCSSDSDSSFTQNLPELPPMVTFDIADVERDGEGKCEENPEFHNDEDLAASLEAFELGYYQKHAFNNYRSRFYQGLPWGVSSLPRYLGLPGMHPRPPPAAMALSRRSRSLDTAETLELELSNSHLTQGYMESDELQAQQEDSEEEEEEEEWGRDSPLSLYTEPPVTYDWPAWAPCPLPVGPGHAWISPSQLDGPSSHPFGQAVCCISPFTMSMLLSVSGPEPRAPGESKSQLARPSHLPLPMGPCYNLQPKASQSVRARPQDVLLPVDEPSCSSISGGFSPSPVPQAKPVGITHGIPQLPRIRPEPPEPQPIHYGASRLDLSKEKAEQGASLPTSCSSTAMNGKLAE

SEQ ID NO: 39 - DNA, 3596 bp, Rattus norvegicus
atgggggagctgctcagagcctggagaagggtagtcggggcaggagactgttgtagtcctggtgggggctgctgcaggagcagtggtggcggcgttctcagcagaggttccagctcagatccgagcagcgcagcagacaataaccgcgctgggagcaggacctggcaggaccccactgtgatgcttcctgactgatcatgttataagtgtgcctggaatccaaaggcagcgtttccaattggagagttctcttcatcatcatggagattcaacaggatgaaacagttcaaatcaagaggacatcaacttccagtgatgcccaagatcagggagctgagaagggagccaagaataagacagctgagacaacagaaggatcaacatcagagccacccttatcaggcccaggtaggctgaagaaaactgccttgaaactctttggtggcaagaagggaatctgtactctgcctagtttctttgggggtggacgaagcaaaggttctgggaaaggcagctccaagaagggtctaaacaagagcaagacccacgatggtctgagtgaatcatctcacggccctgaagatgttgtcgttgaagaaacagagctctctacacctttgtccaagtcaccttctcaatttcctagctcccagagtgcccatggagctttggagataggctctagacataagacatctgtgactgaagctgtagagaaagctggagttgagaaggtaccctctgtgcacaagccgaagaaaggcctaaaaggctttttaaacagtatccgccgtcaccgaaagagcaaggtttctggggctgaccaaagtgggctaggggccaaggaacttgaggggacaagaaccatgtctcaagagcatgtgagttcaatctctctgcccagctctgaggagatattccgagatgctataaaggaaaataccaaaccccaggatgcccctggaccaaaaatgtctccagcaagagaacatttttcactaactactgagaagacagcttgtaaaaatccagaaaaacttacaatgacctgtgcctcagagttcatgcagcccaagcctattcttgaagctagtagtctagaagagccccacacttcagaaacagaaggaaaagtggtaacaggagacgcaaacccacccaatggcccggttggggatcagctaagcctcttgtttggggatgttacatccctgaaaagctttgactcgctgacaggttgtggtgacattatagcagaacaggacatggacagtatgacagatagcatggcctctggtggccagagggccaaccgagatgggaccaagcgaagttcctgcttggtgacctaccaaggaggtggggaggaaatggccttgccagatgacgatgacaatgaagaggaagaagaagaggatgaggaggtagaattagaggatgaggaagaggaggtcaaggatggggaggaagatgatgacttagagtatctgtgggcaagtgcccagatatacccaagatttaatatgaacctgggctatcatactgctatatctcccagccaccagggctacatgcttcttgacccagttcagtcttatcctaacctaggcctgggagaacttttgacacctcagagtgaccagcaagagtctgctcccaatagtgatgaaggttattatgactctaccacaccaggatttgaggatgattcgggtgaggccctggggcttgtccatagggattgtctacccagagacagctatagtggtgatgctctttatgaattctatgagccagatgacagtcttgagcactccccacctggggatgactgcctttatgacctccgtggacgcaactcagagatgttggatcctttcttgaacttggagcccttttcttctcgaccacctggggcaatggaaacagaagaagaacggctggtaaccatccagaagcagttgctgtactgggaacttcgccgggagcagcgtgaggctcaagaggcatgtgccagagaggctcatgccagagaagcttattctcgagacacccacactcgggaatcttacggcaaaaatacccgagcccgagaaacccaagccctagaggttcattctcaagagggtcgagtccaagagaccaaagtacggcaagagaagcctgccttggaatatcagatgaggccattaggcccctcggtgatgggtctggtgccagggacaccaggaggttctcagacttcacaccggggaaccacttcagctttcccagctacttccagtagtgaaccagactggcgggatttccgtcctctggagaagcgttttgaggggacttgctccaaaaaagatcaaagtacttgtttaatgcagctctttcagagtgatgccatgtttgaaccagacatgcaagaagcaaatttcggaggctctcccaggaaggcctaccctagttactcaccccctgaagagccagaggaagaggaagatgagaacgaagggaatgccactgtgagtttttcacaagctctagtagagtttaccagcaatggaaacctttttaccagcatgtcctacagctctgattctgactcatccttcactcaaaatctccctgagttgccccccatggtcacctttgacattgctgatgtggaacgggatggagaaggcaagtgtgaagagaatcctgagttcaacaatgatgaagatcttacggcctccttggaagcttttgagctgggttattaccacaaacatgccttcaacagctatcacagccggttctaccaaggcctgccctggggtgttagcagcctccctcgatacttaggactcccgggtgtacatcctcgacctccacccgctgcaatggcactcaataggagaagccgttcccttgacaatgcagagaccctggagctggagctctccagttctcacctagcccagggttatgtggagtctgatgagctacaggaccaccaggaagattcagatgaagaaggtgaagaagaggaaggagaatggggccgggacagccccttgtcactctacactgaacctccaggggtctatgactggccaccctgggctcactgtcctgttccagtgggaccaggtcttgcttggctaagccctaatcagttggatgagcctttcaatcagtcttcatatgtgcaagcaacctgttgtgtacctcctgtggcaatgtcagtgccaggaagagcacctggagactctgtgtctcagccagctcgaccttcacacctacccctgcccatgggtccttgctataaccttcaatcacaggcctcccagagtgggagggccaggcctcgggatgtactgctgcctgtagatgagcccagttgctcctccatttctggagccaactctctgtctcaagccaagcctgtaggcatcacccatggtatccctcagctgcccagggtacggcctgaaccccttcagcttcaacccaatcactaccgagcttccaaccttgacctttcaaaggagaggggtgaacaaggtgcctctctctctaccagctactcctccactgccatgaatggaaacctagccaagtag

SEQ ID NO: 40 - protein, 1111 aa, Rattus norvegicus
MEIQQDETVQIKRTSTSSDAQDQGAEKGAKNKTAETTEGSTSEPPLSGPGRLKKTALKLFGGKKGICTLPSFFGGGRSKGSGKGSSKKGLNKSKTHDGLSESSHGPEDVVVEETELSTPLSKSPSQFPSSQSAHGALEIGSRHKTSVTEAVEKAGVEKVPSVHKPKKGLKGFLNSIRRHRKSKVSGADQSGLGAKELEGTRTMSQEHVSSISLPSSEEIFRDAIKENTKPQDAPGPKMSPAREHFSLTTEKTACKNPEKLTMTCASEFMQPKPILEASSLEEPHTSETEGKVVTGDANPPNGPVGDQLSLLFGDVTSLKSFDSLTGCGDIIAEQDMDSMTDSMASGGQRANRDGTKRSSCLVTYQGGGEEMALPDDDDNEEEEEEDEEVELEDEEEEVKDGEEDDDLEYLWASAQIYPRFNMNLGYHTAISPSHQGYMLLDPVQSYPNLGLGELLTPQSDQQESAPNSDEGYYDSTTPGFEDDSGEALGLVHRDCLPRDSYSGDALYEFYEPDDSLEHSPPGDDCLYDLRGRNSEMLDPFLNLEPFSSRPPGAMETEEERLVTIQKQLLYWELRREQREAQEACAREAHAREAYSRDTHTRESYGKNTRARETQALEVHSQEGRVQETKVRQEKPALEYQMRPLGPSVMGLVPGTPGGSQTSHRGTTSAFPATSSSEPDWRDFRPLEKRFEGTCSKKDQSTCLMQLFQSDAMFEPDMQEANFGGSPRKAYPSYSPPEEPEEEEDENEGNATVSFSQALVEFTSNGNLFTSMSYSSDSDSSFTQNLPELPPMVTFDIADVERDGEGKCEENPEFNNDEDLTASLEAFELGYYHKHAFNSYHSRFYQGLPWGVSSLPRYLGLPGVHPRPPPAAMALNRRSRSLDNAETLELELSSSHLAQGYVESDELQDHQEDSDEEGEEEEGEWGRDSPLSLYTEPPGVYDWPPWAHCPVPVGPGLAWLSPNQLDEPFNQSSYVQATCCVPPVAMSVPGRAPGDSVSQPARPSHLPLPMGPCYNLQSQASQSGRARPRDVLLPVDEPSCSSISGANSLSQAKPVGITHGIPQLPRVRPEPLQLQPNHYRASNLDLSKERGEQGASLSTSYSSTAMNGNLAK

SEQ ID NO: 41 - DNA, 3432 bp, Gallus gallus
atggaaacaggctgcacggaggaggctgccaggacccagtcgcagtctgcagcatgcaggcagtgcaagggcggtgagcagcagccagaggagaacgttgagcggctgtcccagcggagtgatgcctcagccacggctgcagagcagcagccgccccaggcaccctctggcaagctgaagaaaactgctttcaagctatttggagggaagaggagtatatgtacactgccaagtttcttcagcagcagaaacaaaggccaggggaaaggagcttctaaaaaggggctcagtaaaagtaagacccacgatgggatcagcggcactgcatatgatgagggtagtggagtgcagttggaaagcccttcagatgggagcagggattctcatccttgcctgctgccaagttcccaaagtgttcacgtggccatagacaccagcgtcaagtttgattttggcaggcaggatggctctcctccagggagtattgagggctatgagaagaagcccaacggagacaagtcatccttccccagaccgaaaaaaggccttaaggggcttctgaacagcatccggcgtcaccgtaagagcaaggttgctgagtgtgagaaaacagagctctctgagtggcctggggattcagaggagaccagcaaagctcagggcacaaaagcggaaacccctggaactgtggaggaaggagtacctggatctgtccctcttgctgcagcctgcccagggagctcagaggataactgcttggtgcgcacagtagctgactttggggaggctgctgagccagactggctccaggctgataagggcagctgtgaaggcgatgtggtagcagtgcctgggggcaaggatgatctggatgcaaaatcagaggtcgatgctgttgtctacacagagtccaactacagccacctccctgtggcccttcatccagacttagcagacaatgaccctccctcattacattctggggaccagctgagtctcctctttggagacgtcacatccctgaagagctttgattccctcacagggtgcggagacatcattgccgagcctgacatcctcagcattgctgagagcacgatctctgtggagcgcagccgagacactgctaaacggagcccatgcctcgtcacctaccagggaggtggggaggaaatggccatgtctgaggaggagtacctccagcagatatgggacagcaccgctgaagagggcaggagctacgaagccccgctgcctccagttgtgagcagtcccgaactgcaggcagtgagtagcaagttggagacacgtggtttgcatgagggggaggtccacccctatgctagtggggcagtggatggggtcgaactcctgacaccacagagtgaccagcaagagtctgcacctaatagtgatgagggatattatgactccaccacaccaggaccagaggatgagactggggatggactttgtgagatcaagaaggaccgtcttcccagagacagctacagtggcgatgcactttatgagttttacgagcccgatgatacactcatgagcccctctcatggggagcaatccctgtttgagagcaaagtctctcatccagagatcttcagctacttcttggacttctgcctccctcctgagaagagcttgatccagatgatggatcagaagaggggggttatggaaacggaagaagagcggctagctgccattcagaaagagctgctgttttgggagctgcagagggaaccaggcttgaaacgacttgatgttcccagcaaagagaagtgtcccagggaaaagcagcacgttgaatgtaatactagagcagccaacttgattggcaaaagtcagagttgccttggtagtgagcagattgcctctcaagctctaaacaggagtgtgaatggtgggattttggtggctagagctgaaaatccagagtggagtgattttccagggcgtttatgtccagagaactgttacaacagccaaaaagcccaaggaagttgccttattcagctcatgaagaacaacccagggttcgattctgacccagactgtgcattgtttggggggtccctccatggcagtgcagccttggccaaaccggggatgttccccagccgcagacccctggagcatgagggctgcttgcagagcgagcaccacagtggggtggaggctgccggcagagagcctcaggctgacagtgagtgtgaacctgagcacgccatgagcttctcacaagctctggtggagttcaccagcagtgggaccctcttctccagcctgtcggaaagcctgggcagctccgactctggctcttccttcactcagaacctccccgtccttcccaccatggtcacgtttgacattgtcgatgtggagcaggaaggggaaggggagtgcgaacagcacctcgagatgaatgcggatgaggacatggctgcctccttcgaggccttcgatgagagctacgtgcagaaggagtcatttgctgaatgtgacgagagaatgttcctggggtacccccagggctccttccagagctgtaactggggggttgccagcctgccccggcacctgcatctgcacgggctcagccctgccatgccagcaccgctctcccttaacaggaggagcaggtcacttgacacggagagcctggagtttgaacttgcggacttgcaggtttccaagaatggccttaagccttgccagctgtggtcaaaatgggacaaaaagtactcagatggagcgagaaggagcaggagcaaagaggaggcagaactgtcagctcctgaaggtggggagaccaatggtgtgctgagctggccgggcttgcagcaccttcagtacgatgccgagctggctcctggggggatcaaacactggggttttgctccagctgctgggatgccgagcagctgggagccatctgagcggtcagatgcagacccctcattttttcccctgtcaaggagctgtgccagagaaactctggagaggcagcctcaagatccggagccaaacaggctcctggtcaggccatccaacttacctctgcagcctgacacgaggcagccccaggaggcgtctgggtcatacaggtatcacggagaagccccggccaaaaagctcgcacgtgtgctcccgctgggggaaccagagccaccgcagagcatcagctttgcccagtccccagagaaaccagccaagtgcaaacctgtcggcgtcacgcaaggaatgccccagtgccacgacagcgatgacaccgaaaccttaaaaagctcagcctgctttgcagagcgctacgggagtaccagcaaagagctgctgaaagcaagagctgcgccggggaatgtgctgcccagcggctgcctgagcactgcagtgaatgtaatgggagccaaatag

SEQ ID NO: 42 - protein, 1143 aa, Gallus gallus
METGCTEEAARTQSQSAACRQCKGGEQQPEENVERLSQRSDASATAAEQQPPQAPSGKLKKTAFKLFGGKRSICTLPSFFSSRNKGQGKGASKKGLSKSKTHDGISGTAYDEGSGVQLESPSDGSRDSHPCLLPSSQSVHVAIDTSVKFDFGRQDGSPPGSIEGYEKKPNGDKSSFPRPKKGLKGLLNSIRRHRKSKVAECEKTELSEWPGDSEETSKAQGTKAETPGTVEEGVPGSVPLAAACPGSSEDNCLVRTVADFGEAAEPDWLQADKGSCEGDVVAVPGGKDDLDAKSEVDAVVYTESNYSHLPVALHPDLADNDPPSLHSGDQLSLLFGDVTSLKSFDSLTGCGDIIAEPDILSIAESTISVERSRDTAKRSPCLVTYQGGGEEMAMSEEEYLQQIWDSTAEEGRSYEAPLPPVVSSPELQAVSSKLETRGLHEGEVHPYASGAVDGVELLTPQSDQQESAPNSDEGYYDSTTPGPEDETGDGLCEIKKDRLPRDSYSGDALYEFYEPDDTLMSPSHGEQSLFESKVSHPEIFSYFLDFCLPPEKSLIQMMDQKRGVMETEEERLAAIQKELLFWELQREPGLKRLDVPSKEKCPREKQHVECNTRAANLIGKSQSCLGSEQIASQALNRSVNGGILVARAENPEWSDFPGRLCPENCYNSQKAQGSCLIQLMKNNPGFDSDPDCALFGGSLHGSAALAKPGMFPSRRPLEHEGCLQSEHHSGVEAAGREPQADSECEPEHAMSFSQALVEFTSSGTLFSSLSESLGSSDSGSSFTQNLPVLPTMVTFDIVDVEQEGEGECEQHLEMNADEDMAASFEAFDESYVQKESFAECDERMFLGYPQGSFQSCNWGVASLPRHLHLHGLSPAMPAPLSLNRRSRSLDTESLEFELADLQVSKNGLKPCQLWSKWDKKYSDGARRSRSKEEAELSAPEGGETNGVLSWPGLQHLQYDAELAPGGIKHWGFAPAAGMPSSWEPSERSDADPSFFPLSRSCARETLERQPQDPEPNRLLVRPSNLPLQPDTRQPQEASGSYRYHGEAPAKKLARVLPLGEPEPPQSISFAQSPEKPAKCKPVGVTQGMPQCHDSDDTETLKSSACFAERYGSTSKELLKARAAPGNVLPSGCLSTAVNVMGAK

SEQ ID NO: 43 - DNA, 2862 bp, Danio rerio
atggagatcgccacaagatgtgaggttggtgcaatgaggggtccatcgtcagactccgtttcccatgatatccctcaaccacagtcacccccttctgtgaaaatacggaagacagctttcaaattttttggaggtcgcaaaagcatatgtgttctacctagcttttttggaggccgtggcagaagtcaaagaaaggggtcatcaaaaacaggagtaacaaaaagccagacatatgatggggtgagccgagcatgctgggatgatttgggtagaagtagcagtgaagtggcatcaggggactttgagttctgcagtgaacctcagaaatcacaggaggatcatgggaagtcacagtctttgcctagacagcgcaggggccttcgaggacttttcagcagcattcggagacacaggaaaaacaaaaatgttgaagtagaaaagcgggaagcacttgaaatgtcttcaagcttccatgccaaaacagtgcctggtgccctgccgagtgtcagtgaccgtggcgattaccatggagacagtcagggtgaagagctggtgccagatgtgcctaatcaaaccacaggcagtgagtgtgaactgcctttggctgccactgaatgtacgatagatgtaacactagtgcctgaaaaaaggaggagcagagtagagatggataagaggaggagagcagaagaagaggggattggagaagatgagaaaactggcagacaggaaggactgatgacatatcatcaacccttgtctgctgagtctgaattggatcgtttggctgagcaaaatgtggacgttcctgatggagagccacctgttgcatcctgctcctctgagaacttagtctttggtgatgtgtcatcattaaagagctttgattcgctgaccggttgtggagatatcattgcagaccaggatgatgtcagtgtcgccgagagctcagtgtctgctgacagaggcagtcgaaatgcaggaaaacgtagctcatgttttgtcacatatcaaggtgggggggaagagatggcaaccccggatgagatagacgcggattacttgcagagcttatgggagtcagaaactagtaatgaagtctgctacatcccctcagataggggctcagatagcccttcactcacccctgatcagcaactgagctctatccgtgccacctccagcagcagcccaatgggaatcacagaaactgctctcactcctgccgacctcttgagtccacagagtgatcggcaggagtcggtacccaacagtgacgagggttactatgactccaccacgccagggatggaagaggagagtagagaacgtccacaccaggaaaggcttccacgggacagctacagtggtgatgccctttatgaactttttgagcctgacgatcgcctgctcagtccttctcttcctcccaaggatgcccactcatttgttggtgcacccttgcaggctgataagagtccaacaaacccgctatactccttggcatctactgcaatagaaacaggagccatggagacagaggaggagcgcctgagcaagatccaacatgcccttctgtgctgtgagcttcagaatctcaggagtccatccaaaaaccagcttttgttccattccgactgcttttatgatgattcgaaccttccagttgatgatagtaagcaagatttgcaagaagttatcaatcagcgctatccccaatccccaccaagatcccaggctgttaaagagggagtccctcgcatcaggggtcaggttcaagagagttcgttattcgcaccctgtgctgattcggtgttgaatccacaagtcatcgagacaaccagaccgcagcctcaatctgacgatcaaggctcattacgaccctccagaggctgcagccaatcccaagaagagctgatggtctgtttctcccaagccttggtagattttacaaagaacacccggctttaccgcaattcaactgaaagtcttgatggctctgagtcaagttctccctttggcccaagtctgagagccctgcctgccattgtaacattcgatgtggttgatatggagaatgagggtgagtgtgagcagcagactgaccttgcagaggaagaggaagagttggcatctccttatgagccattcgaagatgatggctgttaccttcaacaagatgcctttgctgagtgtgaccagcgtacttttgatgcttatgaacagagtctgctgctcagcaatgcttggggaattgcaagccttcctcggcacctcagtttgggtcgaccttgtccaccagtccctgcccctttagcactaaatcgccgaagtcgctcacttgacacagacagtctggagtttcagacaagtgaaatttacacaagcgtaaccaagtatgactcaaaaggcactgcattttcacagagcaggactgttgattgtaatgatatggattttccacgacaaccctgcagaattacggttgacagctggaggaggggctacagacagaattttgattccagcaatgcttctcagcaagaactgaagttgccacatttgagccaatccactgtcagaccttcacacctgcccctaaagaacaattgccgtagtcgtaacctccctgctgccactagagtggatggtgagggggagattttatttggtggaggcgatgcgctttatccgtgcagttacccccctatgggcacacagtggaagaatcggcctgtcggggtcacccagggtgtgccacatcttcgctctgagcaatctgcagaccatcaagaaatcacaatgaagaacaggaggggggaacttaagggtggcttcacgcctgcaccccccaagttatga

SEQ ID NO: 44 - protein, 953 aa, Danio rerio
MEIATRCEVGAMRGPSSDSVSHDIPQPQSPPSVKIRKTAFKFFGGRKSICVLPSFFGGRGRSQRKGSSKTGVTKSQTYDGVSRACWDDLGRSSSEVASGDFEFCSEPQKSQEDHGKSQSLPRQRRGLRGLFSSIRRHRKNKNVEVEKREALEMSSSFHAKTVPGALPSVSDRGDYHGDSQGEELVPDVPNQTTGSECELPLAATECTIDVTLVPEKRRSRVEMDKRRRAEEEGIGEDEKTGRQEGLMTYHQPLSAESELDRLAEQNVDVPDGEPPVASCSSENLVFGDVSSLKSFDSLTGCGDIIADQDDVSVAESSVSADRGSRNAGKRSSCFVTYQGGGEEMATPDEIDADYLQSLWESETSNEVCYIPSDRGSDSPSLTPDQQLSSIRATSSSSPMGITETALTPADLLSPQSDRQESVPNSDEGYYDSTTPGMEEESRERPHQERLPRDSYSGDALYELFEPDDRLLSPSLPPKDAHSFVGAPLQADKSPTNPLYSLASTAIETGAMETEEERLSKIQHALLCCELQNLRSPSKNQLLFHSDCFYDDSNLPVDDSKQDLQEVINQRYPQSPPRSQAVKEGVPRIRGQVQESSLFAPCADSVLNPQVIETTRPQPQSDDQGSLRPSRGCSQSQEELMVCFSQALVDFTKNTRLYRNSTESLDGSESSSPFGPSLRALPAIVTFDVVDMENEGECEQQTDLAEEEEELASPYEPFEDDGCYLQQDAFAECDQRTFDAYEQSLLLSNAWGIASLPRHLSLGRPCPPVPAPLALNRRSRSLDTDSLEFQTSEIYTSVTKYDSKGTAFSQSRTVDCNDMDFPRQPCRITVDSWRRGYRQNFDSSNASQQELKLPHLSQSTVRPSHLPLKNNCRSRNLPAATRVDGEGEILFGGGDALYPCSYPPMGTQWKNRPVGVTQGVPHLRSEQSADHQEITMKNRRGELKGGFTPAPPKL

SEQ ID NO: 45 - RNA, 25 nt, Artificial
  sense
agggaaucuguacucugccuaguuu

SEQ ID NO: 46 - RNA, 25 nt, Artificial
  antisense
aaacuaggcagaguacagauucccu

SEQ ID NO: 47 - RNA, 25 nt, Artificial
  sense
gcaacucagagauguuggauccuuu

SEQ ID NO: 48 - RNA, 25 nt, Artificial
  antisense
aaaggauccaacaucucugaguugc

SEQ ID NO: 49 - RNA, 25 nt, Artificial
  sense
aggucuacauggucuauccgaguuu

SEQ ID NO: 50 - RNA, 25 nt, Artificial
  antisense
aaacucggauagaccauguagaccu

SEQ ID NO: 51 - RNA, 25 nt, Artificial
  sense
gcaacugagguaguuuagccacuuu

SEQ ID NO: 52 - RNA, 25 nt, Artificial
  antisense
aaaguggcuaaacuaccucaguugc

(19) United States
(12) Patent Application Publication
(10) Pub. No.: US 2011/0251130 A1
(43) Pub. Date: Oct. 13, 2011

(54) Methods and Compositions for Promoting Bone Growth

(76) Inventor: Stephen Peter Robertson, East Taieri (NZ)

(21) Appl. No.: 13/001,614

(22) PCT Filed: Jul. 7, 2009
(86) PCT No.: PCT/NZ2009/000134

§ 371 Date: Jun. 9, 2011

Related U.S. Application Data
(60) Provisional application No. 61/078,843, filed on Jul. 8, 2008.

(51) Int. Cl. A61K 38/00; A61K 31/7088; C12N 15/85; A61P 19/00; C12N 5/077; A61K 31/713; C07H 21/02

(52) U.S. Cl. 514/16.7; 514/44 A; 514/44 R; 536/24.5; 435/375; 435/455

(57) ABSTRACT

The invention relates to methods and compositions for promoting bone growth, bone healing and/or bone formation. Disclosed herein are isolated truncated Wilms tumor on gene chromosome X (WTX) polypeptides and uses thereof. Also disclosed are methods of treating a bone related disease or condition in a subject by modulating the expression or activity of a (WTX) polypeptide in bone cells of the subject. The bone related disease or condition is any disease or condition including bone related trauma or injury due to any cause including surgery. The compositions and methods according to the invention are useful for enhancing and stimulating bone mineralization and for preferentially directing the differentiation of certain stem cells, thereby altering cell fate.

### FIELD OF THE INVENTION

This invention relates to methods and compositions for promoting bone growth or differentiation. Such methods and compositions can be used in vitro, or in vivo for treating bone related diseases or conditions.

### BACKGROUND

Bone related disease and conditions due to injury or resulting from surgical procedures are common. The successful treatment of bone injuries would be greatly improved by methods that increase the speed of bone healing and/or the quality of bone formed during the healing process. In particular, numerous bone-related surgical procedures, for example, treatment of bone breakage or bone fracture, bone grafts, dental implants, bone re-alignments, bone instrumentation, and joint replacement, are routinely performed which require either bone healing or bone formation or both, for successful outcomes.

Current methods of treating bone trauma and bone injury are typically limited to methods that employ the body's natural ability to promote bone growth and formation at the site of trauma or injury. Alternatively, costly procedures which utilize reinforcing pins, plates or other braces may be required following various types of bone repair or remodeling. Such procedures will typically require additional surgery to remove reinforcing pins, plates or other braces. Additional costs associated with current methods include any loss of productivity of individuals that are incapacitated due to the length of time required for the natural healing process and to time lost for re-treatment or additional surgical procedures.

Moreover, the natural process of bone healing is subject to the vagaries of an individual's innate ability to respond to treatment and can be of extended duration. The rate of natural healing may vary considerably amongst treated individuals and can be influenced by conditions such as genetic predisposition, diet, age and other factors. During this time, the individual runs the continual risk of aggravating the treatment site or of sustaining further trauma or injury. In addition, certain anatomic sites heal poorly in comparison to other sites, e.g., ankles. The most common reason for the failure of a joint prosthesis is loosening, which is a function of bone quality, i.e., bone density, composition and regenerative capacity.

Consequently, there is a need for alternative methods of promoting bone healing, bone growth and/or bone formation. It is an object of the present invention to provide improved compositions and methods that can be used to increase at least one of bone growth, bone formation and bone quality and/or to at least provide the public with a useful choice.

### SUMMARY OF THE INVENTION

The applicants have discovered that germline mutations in the gene WTX (Wilms tumor gene on the X chromosome) lead to an X-linked sclerosing bone dysplasia, osteopathia striata congenita with cranial sclerosis (OSCS; OMIM 166500). This condition is characterised by increased bone density and craniofacial malformations. WTX is a cancer associated protein that is a component of the β-catenin destruction complex. WTX protein has been shown to be somatically inactivated in 18-29% of cases of Wilms tumour. Additionally, wild type WTX exhibits tumor suppressor activity in an osteosarcoma cell line¹⁰.

An important and surprising discovery made by the applicants is that despite identical mutations occurring in the germline of individuals with OSCS and somatically in Wilms tumors, individuals with OSCS do not demonstrate a predisposition to tumor development. This observation is at odds with established understanding of tumor suppressor gene function and, without wishing to be bound by any theory, suggests the existence of temporal or spatial constraints on this action of WTX during tumorigenesis.

The applicants have shown for the first time that two isoforms of the WTX protein are expressed in normal individuals due to alternative splicing. The applicants have further shown that germline mutations together with the alternative splicing result in several novel truncated WTX polypeptide fragments. The applicants have also shown that WTX is highly expressed in the fetal murine skeleton and that the alternate splicing implicates subcellular localization and retention of an adenomatous polyposis coli binding domain (APCBD) and acidic domain (AD) as factors conferring survivability in males with OSCS.

The applicant's discoveries give rise to numerous aspects of the invention relating to promoting bone growth, healing and formation and for improving bone density by modulating WTX polypeptide expression or activity.

The present invention provides truncated forms of the full length wild type WTX polypeptide useful for promoting bone growth and formation via modulation of WTX gene expression, or activity, or both. The present invention also provides methods for promoting bone healing by stimulating or enhancing bone formation in a subject via modulation of WTX gene expression or activity or both. The inventive methods can be used to treat a subject that has sustained bone trauma or bone injury, including bone trauma or injury due to an accident or a medical procedure.

Accordingly, in a first aspect the invention provides an isolated truncated “Wilms tumor gene on the X chromosome” (WTX) polypeptide
(a) from which at least five consecutive amino acids present in the full length wild type WTX polypeptide between amino acid residue 50 and amino acid residue 327 are absent, or
(b) consisting of a fragment of at least five consecutive amino acids present in amino acid residues 1 to 545 of the full length, wild type WTX polypeptide.

Preferably a truncated WTX polypeptide is capable of modulating the activity of WTX in a cell.

Preferably a full length wild type polypeptide comprises the amino acid sequence of SEQ ID NO: 2

Preferably a full length wild type WTX polypeptide consists of an amino acid sequence of SEQ ID NO: 2.

Preferably a truncated WTX polypeptide comprises the contiguous amino acid sequence of SEQ ID NO: 4.

Preferably a full length WTX polypeptide comprises the amino acid sequence of SEQ ID NO: 24.

In one embodiment the isolated truncated WTX polypeptide of the invention consists of
(a) the amino acid sequence of SEQ ID NO: 4, SEQ ID NO: 6, SEQ ID NO:8, SEQ ID NO: 10 or SEQ ID NO: 12, SEQ ID NO: 14, SEQ ID NO: 16, SEQ ID NO: 18, SEQ ID NO: 20, SEQ ID NO: 22 or SEQ ID NO: 24, or
(b) an amino acid sequence at least 75% identical to the amino acid sequence in (a).

The invention also provides a fusion protein comprising a truncated WTX polypeptide of the invention.

In one embodiment, a truncated WTX polypeptide of the invention comprises at least a β-catenin binding domain. In another embodiment, a truncated WTX polypeptide of the invention comprises at least a plasma membrane localization domain. In yet another embodiment, a truncated WTX polypeptide of the invention comprises at least a nuclear localization domain.

The invention also provides an isolated polynucleotide that,
(a) is capable of expressing a truncated WTX polypeptide of the invention or
(b) is a nucleotide sequence that is at least 75% identical to the nucleotide sequence of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23.

In a particular embodiment, the invention provides an isolated polynucleotide of the invention which consists of the nucleotide sequence of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23.

The invention also provides an expression cassette comprising,
(a) a polynucleotide of the invention
(b) fragments of 8 to 80 contiguous nucleotides of the polynucleotide sequence in (a), or
(c) a polynucleotide complementary to the polynucleotide sequence in (a) or (b), wherein the polynucleotide in any of (a) to (c) is under the control of a promoter.

In another embodiment, the invention provides a vector that comprises an expression cassette of the invention. Preferably the vector is a recombinant plasmid or a recombinant viral vector.

The invention also provides an isolated host cell comprising an expression cassette or vector of the invention. In another embodiment the invention provides a cell culture comprising the host cell of the invention. In yet another embodiment, the invention provides a non-human transgenic animal comprising an expression cassette, vector or host cell of the invention.

Another aspect of the invention provides a method of making a polypeptide of the invention comprising the steps of transforming a host cell with an expression cassette or vector of the invention, culturing the host cell under conditions whereby the polypeptide is expressed and subsequently isolating the expressed polypeptide. In a particular embodiment, the host cell transformed in the method of making the polypeptide of the invention is comprised in a non-human transgenic animal.

In a further aspect the invention provides use of a truncated WTX polypeptide or polynucleotide of the invention as a diagnostic or prognostic marker.

In one aspect, the invention provides a method of treating a bone-related disease or condition in a subject or animal, the method comprising administration to a subject in need thereof, an agent that modulates the expression or activity of a WTX polypeptide in the subject.

In another aspect, the invention provides a method for treating a bone-related disease or condition in a subject, the method comprising administration to a subject in need thereof, an agent that modulates the expression or activity of a WTX associated signaling pathway in the subject.

In a further aspect, the invention provides a method of promoting bone healing or bone formation, the method comprising contacting cells with an agent that modulates the expression or activity of a WTX polypeptide.

In another aspect, the invention provides a method of modulating osteoprogenitor cell differentiation, the method comprising contacting osteoprogenitor cells with an agent that modulates the expression or activity of a WTX polypeptide.

In yet another aspect, the invention provides a method for increasing or stimulating osteoblast differentiation, the method comprising administration to multipotent progenitor cells of an agent that modulates the expression or activity of a WTX polypeptide.

In a further aspect, the invention provides a method for stimulating or enhancing mineralization of matrix by cells, the method comprising administration to cells of an agent that modulates the expression or activity of a WTX polypeptide.

Preferably the agent is administered to a subject in an effective amount or cells are contacted with an effective amount of the agent. More preferably the agent is administered to a subject in a therapeutically effective amount.

Preferably the methods of the invention include the step of providing a patient that has been identified as having a bone-related disease or condition.

Preferably the bone-related disease or condition is osteoporosis.

Preferably the bone-related disease or condition is a bone trauma or bone injury, including bone trauma or injury due to accident or any medical or surgical procedure.

In one embodiment, the agent used in the methods of the invention is any agent that inhibits the expression or activity of a WTX polypeptide.

Preferably the agent used in the methods of the invention is selected from the group consisting of:
(i) an antibody capable of specifically binding to a WTX polypeptide,
(ii) an antisense compound that inhibits the expression of said WTX polypeptide,
(iii) an expression vector encoding said antisense compound,
(iv) a dsRNA that inhibits the expression of said WTX polypeptide,
(v) one or more expression vectors encoding said dsRNA,
(vi) a ribozyme that inhibits the expression of said WTX polypeptide,
(vii) one or more expression vectors encoding said ribozyme,
(viii) a fragment of a WTX polypeptide,
(ix) a truncated WTX polypeptide of the invention,
(x) an expression vector comprising a polynucleotide which encodes a polypeptide of the invention,
(xi) a fusion protein comprising a truncated WTX polypeptide of the invention,
(xii) a small molecule inhibitor of WTX gene expression or activity,
(xiii) an aptamer that inhibits WTX gene expression or activity, and
(xiv) a peptide nucleic acid that inhibits WTX gene expression or activity.

Preferably the useful agent is a dsRNA, an antisense compound, an antibody, a WTX polypeptide fragment or variant, a truncated WTX polypeptide, ribozyme, an aptamer or a small molecule inhibitor.

More preferably the useful agent is a dsRNA, siRNA, antisense compound, ribozyme or small molecule inhibitor. More preferably the useful agent is a siRNA, shRNA, antisense oligonucleotide or small molecule inhibitor.

Preferably, the dsRNA is an isolated siRNA or shRNA comprising a sense RNA strand and an antisense RNA strand, wherein the sense and the antisense RNA strands form an RNA duplex, and wherein the sense RNA strand comprises a nucleotide sequence that is substantially identical to a target sequence of about 18 to about 25 contiguous nucleotides of a WTX mRNA. More preferably, the sense RNA strand comprises a nucleotide sequence that is identical to the target sequence.

Preferably, the dsRNA is a siRNA or shRNA that targets and specifically hybridizes to a human WTX mRNA comprising SEQ ID NO: 1. More preferably, the siRNA or shRNA targets and specifically hybridizes to SEQ ID NO: 1 between nucleotide residues at positions 150 and 981.

In another embodiment, the dsRNA is an siRNA or shRNA that targets and specifically hybridizes to a mouse WTX mRNA comprising SEQ ID NO: 25. In a preferred embodiment, the siRNA or shRNA target and specifically hybridizes to SEQ ID NO: 25 between nucleotide at positions 150 to 981.

In one embodiment, the siRNA or shRNA is selected from the group consisting of an siRNA or shRNA having a sense strand that comprises SEQ ID NO: 29 and an antisense strand that comprises SEQ ID NO: 30 and an siRNA or shRNA having a sense strand that comprises SEQ ID NO: 31 and the siRNA antisense strand comprises SEQ ID NO: 32.

In a preferred embodiment the siRNA comprises a sense strand comprising SEQ ID NO: 45 and an antisense strand comprising SEQ ID NO: 46 or a sense strand comprising SEQ ID NO: 47 and an antisense strand comprising SEQ ID NO: 48. Most preferably the siRNA comprises a sense strand consisting of SEQ ID NO: 45 and an antisense strand consisting of SEQ ID NO: 46 or a sense strand, consisting of SEQ ID NO: 47 and an antisense strand consisting of SEQ ID NO: 48,

Preferably the antisense compound is an antisense oligonucleotide. Preferably the antisense oligonucleotide is a short single stranded DNA. Preferably the antisense oligonucleotide is chemically modified.

Preferably the use of an agent in the methods of the invention inhibits the expression or activity of substantially all WTX polypeptide isoforms that would be expressed or active in a particular cell, tissue-type or tissue.

Preferably the useful agent inhibits the expression or activity of all WTX polypeptide isoforms that would be expressed or active in a particular cell, tissue-type or tissue.

In another embodiment, the useful agent increases the ratio of a splice isoform of WTX polypeptide relative to a full length wild type WTX polypeptide.

Preferably the splice isoform is a shorter splice isoform. Preferably a shorter splice isoform comprises a contiguous sequence of amino acid residues that are fewer contiguous amino acid residues than found in a full length wild type WTX polypeptide due to a deletion of any number of contiguous amino acid residues from either the C-terminal end, N-terminal end or internally, of a WTX polypeptide.

Preferably a shorter splice isoform of a WTX polypeptide comprises an isolated truncated WTX polypeptide from which at least five consecutive amino acids present in the full length wild type WTX polypeptide between amino acid residue 50 and amino acid residue 327 are absent.

Preferably a shorter splice isoform of a WTX polypeptide is the WTXS2 polypeptide as described herein. More preferably the WTXS2 polypeptide consists of SEQ ID NO: 3.

Preferably use of an agent or a WTX inhibitory agent in the methods of the invention results in increased expression or activity of a short isoform of a WTX polypeptide, or fragment or variants thereof, as compared to a full length, wild type WTX polypeptide isoform.

Preferably, selective increase in the transcription of one WTX mRNA as compared to a variant or alternative WTX mRNA transcribed from the same gene is achieved.

Preferably, selective increase in transcription of one WTX mRNA as compared to a variant or alternative WTX mRNA transcribed from the same gene is achieved through the use of a first agent and a second agent wherein the first agent inhibits one WTX mRNA transcribed from the gene and the second agent increases the expression of a variant or alternative WTX mRNA transcribed from the same gene.

More preferably, the first agent inhibits the expression or activity of a full length wild type WTX polypeptide and the second agent promotes expression or activity of a truncated WTX polypeptide of the invention. Most preferably, the first agent is a dsRNA, antisense compound, or ribozyme that is a WTX inhibitory compound and the second agent is a recombinant vector that directs the overexpression of a truncated WTX polypeptide of the invention.

In one embodiment, the selective increase in the transcription of WTX one mRNA as compared to a variant or alternative WTX mRNA transcribed from the same gene is achieved in a cell, in vitro. Preferably the cell is a bone cell, more preferably an osteoprogenitor cell, a multipotent progenitor cell or an osteoblast cell. Preferably the selective increase in mRNA transcription as above is achieved in vivo.

In one embodiment, the agent used in the methods of the invention is formulated in a composition with a pharmaceutically acceptable carrier or diluent.

Preferably, the agent used in the methods of the invention is an inhibitory nucleic acid agent or a small molecule inhibitor.

In one embodiment, an agent used in the methods of the invention is administered in conjunction with a delivery reagent. In one embodiment, the delivery reagent is a molecule, molecular structure or mixture of molecules or compounds that is admixed, used to encapsulate, conjugated to or otherwise associated with an agent that is used in the methods of the invention.

In another embodiment, the methods of the invention further comprise the administration of an additional agent. The further therapeutic agent can be any appropriate therapeutic agent used to treat or prevent any symptom, side effect or other consequence of treatment, either as a result of the use of an agent used in the methods of the invention or for any other reason related to the desired treatment.

In one embodiment, an agent used in the methods of the invention is administered orally, topically or parenterally. Preferably an agent used in the methods of the invention is administered by a direct, intravenous, intradermal, subcutaneous, transdermal, transmucosal or systemic route.

In a particularly preferred embodiment, direct application comprises the direct application, at or near the site of bone fracture or breakage, of a liquid, a paste, a gel or a slurry, or is via a patch, a suppository, an implant comprising a porous material, an implant comprising a non-porous material, an implant comprising a gelatinous material or a coated implant.

Preferably the direct application is local application or local administration.

Preferably administration to a subject, of an agent useful in the methods of the invention, is or results in a transient administration of the agent.

Preferably transient administration of a useful agent comprises the transient expression of an agent or the transient presence of an agent, in a particular cell, cell-type, tissue, tissue-type or particular region of the body of a subject.

Preferably a transient administration is, or results in, administration of a useful agent for a sufficient period of time to provide a treatment or achieve a therapeutic result without the presence of the useful agent resulting in an undesired or harmful effect. Preferably the undesired or harmful effect is tumorigenesis or cancer.

In another aspect, the invention provides the use of an agent, as defined in the methods of the invention, in the preparation of a medicament, composition or formulation for treating at least one bone-related disease or condition.

In another aspect, the invention provides a composition comprising an agent, as defined in the methods of the invention, for use in treating at least one bone related disease or condition.

Preferably the at least one bone related disease or condition is at least one of congenital defects, age-related defects, developmental defects and abnormalities, accidental injuries, drug treatment such as treatment with corticosteroids and conditions requiring or caused by surgical procedures.

Preferably a bone-related disease is osteoporosis including, but not limited to, primary osteoporosis, endocrine osteoporosis (hyperthyroidism, hyperparathyroidism, Cushing's syndrome, and acromegaly), hereditary and congenital forms of osteoporosis (osteogenesis imperfecta, homocystinuria, Menkes' syndrome, and Riley-Day syndrome) and osteoporosis due to immobilization of extremities.

Preferably a bone related condition includes, but is not limited to, a condition that requires or is caused by a bone graft, a bone re-alignment, a bone straightening procedure, a bone lengthening procedure, a bone shortening procedure, a bone replacement procedure, a joint replacement procedure, a bone repair procedure, a bone reconstruction procedure or any other surgical procedure where successful recovery requires at least, one of bone growth, bone regeneration, bone healing or bone formation.

A further aspect of the invention provides a method for identifying an agent which promotes bone healing or bone formation, the method comprising screening one or more candidate agents for the ability to modulate the expression or activity of a WTX polypeptide, wherein the candidate agent that modulates expression or activity of a WTX polypeptide is an agent that promotes bone healing or bone formation.

In another aspect the invention provides a method for identifying an agent which modulates osteoprogenitor cell differentiation, the method comprising contacting an osteoprogenitor cell with one or more candidate agents and assaying the contacted osteoprogenitor cells for modulated expression or activity of a WTX polypeptide as compared to control osteoprogenitor cells that are not contacted with said one or more candidate agents, wherein the candidate agent that modulates expression or activity of a WTX polypeptide is an agent that modulates osteoprogenitor cell differentiation.

In another aspect, the invention provides a method for identifying an agent which increases or stimulates osteoblast differentiation, the method comprising contacting a multipotent progenitor cells with one or more candidate agents and assaying the contacted cells for modulated expression or activity of a WTX polypeptide as compared to control multipotent progenitor cells that are not contacted with said one or more candidate agents, wherein the candidate agent that modulates expression or activity of a WTX polypeptide is an agent that increases or stimulates osteoblast differentiation.

In still another aspect, the invention provides a method for identifying an agent which stimulates or enhances mineralization by a cell, the method comprising contacting a cell with one or more candidate agents and assaying the contacted cells for modulated expression or activity of a WTX polypeptide as compared to control cells that are not contacted with said one or more candidate agents, wherein the candidate agent that modulates expression or activity of a WTX polypeptide is an agent that stimulates or enhances mineralization by a cell.

In one embodiment the agent used in the above methods of screening is any candidate agent that modulates the expression or activity of a WTX polypeptide. In a preferred embodiment, the agent is selected from the group consisting of:
(i) an antibody capable of specifically binding to a WTX polypeptide,
(ii) an antisense compound that inhibits the expression of said WTX polypeptide,
(iii) an expression vector encoding said antisense compound,
(iv) a dsRNA that inhibits the expression of said WTX polypeptide,
(v) one or more expression vectors encoding said dsRNA,
(vi) a ribozyme that inhibits the expression of said WTX polypeptide,
(vii) one or more expression vectors encoding said ribozyme,
(viii) a fragment of a WTX polypeptide,
(ix) a truncated WTX polypeptide of the invention,
(x) an expression vector comprising a polynucleotide which encodes a polypeptide of the invention,
(xi) a fusion protein comprising a truncated WTX polypeptide of the invention,
(xii) a small molecule inhibitor of WTX gene expression or activity,
(xiii) an aptamer that inhibits WTX gene expression or activity, and
(xiv) a peptide nucleic acid that inhibits WTX gene expression or activity.

Preferably screening one or more candidate agents involves measuring in increase or decrease in a WTX polynucleotide or polypeptide expression in vitro or in a cell ex vivo.

Preferably screening one or more candidate agents involves measuring in increase or decrease in a WTX polynucleotide or polypeptide expression in a cell in vivo. The cell in vivo may be in any organism, preferably a non-human organism, more preferably a non-human animal.

Preferably assaying for modulated expression or activity of a WTX polypeptide involves measuring the levels of expression of a WTX polynucleotide or measuring the amount of a WTX polypeptide in a cell as compared to a control cell.

Preferably assaying for modulated activity of a WTX polypeptide involves measuring the levels of binding of a WTX polypeptide to one or more candidate agents. Preferably the level of binding is determined in a cell or in vitro.

Preferably assaying for modulated activity of a WTX polypeptide involves measuring the levels of binding of a WTX polypeptide to another polypeptide to which WTX normally binds. Preferably the level of binding is determined in a cell or in vitro.

Preferably assaying for modulated activity of a WTX polypeptide involves measuring the levels of expression of genes or polypeptides in a WTX associated cell signaling pathway induced by WTX activity.

Preferably assaying for modulated activity of a WTX polypeptide involves measuring the levels of expression of genes or polypeptides involved in osteoprogenitor cell differentiation induced by WTX activity.

Preferably assaying for modulated activity of a WTX polypeptide involves determining the levels of expression of genes or polypeptides that are induced by WTX activity, and involved in stimulated or enhanced mineralization in a cell, more preferably in a bone cell, most preferably in an osteoblast cell.

### BRIEF DESCRIPTION OF THE DRAWINGS

The invention will now be described with reference to the figures.

FIG. 1

Phenotype of the proband (case 06) and delineation of her de novo chromosomal deletion within Xq11. (a) Marked prominence of the forehead, hypertelorism and micrognathia are present; (b) Radiograph of the right leg demonstrating longitudinal linear striations of the long bones; (c) Axial CT scan of the skull demonstrating marked thickening of the skull base and frontal bones; (d) Ideogram of the X chromosome with the centromere marked in red. Beneath is the normalised CNAG data trace from the 500K SNP array with a deletion in the region of Xq11 visible. (e) Diagram of the deleted region adjacent to the X centromere. The solid black line indicates the extent of the deleted region and annotated genes within the deleted and flanking regions; the hatched line, potentially deleted sequences; * denotes the loci for confirmatory qPCR.

FIG. 2

Functional domains within WTX relative to the corresponding positions of point mutations associated with OSCS and Wilms tumor. The WTX protein contains a putative nuclear localisation sequence (NLS; blue), an acidic domain (AD; green) and three APC binding domains (APCBD1-3; red). A proline rich domain lies at the C terminus and the N-terminus possesses phospholipid binding activity. The residues deleted in the alternative spliceform are depicted in yellow and regions of >80% amino acid similarity with the 671 aa paralogous protein encoded by FAM123A, are denoted in light blue. The position of somatic point mutations found in Wilms tumors are denoted with an asterisk. The lengths of predicted truncated WTX proteins produced in males and females with WTX mutations are shown as black bars. The red bars indicate superadded novel polypeptide sequence predicted by mutational frameshift events.

FIG. 3

Expression pattern of WTX in E14.5 murine embryos.

(a)-(e) Sagittal sections through E14.5 dpc embryos stained with a dioxygenin labelled Wtx antisense riboprobe. Staining is identified in (a,b) the ulna (ul); radius (rd) and humerus (hm) of the forelimb (fl) and in the cartilaginous ribs (rb); (c,d) within the skull base (sb), orbit (ob) vertebrae (vb), mandibular anlargen (md) and the pelvis (pv) but not in the mesonephros (mn); (e,f) Expression in the skull is widespread in both the skull base (sb), orbit and nasal bones (nb)·(g−1) Magnified views of boxed regions demonstrating expression within the (g) elbow joint of the forelimb (ol, olecranon) but absent in the ossifying zone within the diaphyses of the ulna (du). Strong staining is noted in the (j) basioccipital bone (bo) and temporal bone complex, and within (i) the calvarium (cv) and (j) skull base (sb). The only strongly staining extraskeletal structures at this gestation were (h) the thymus (th), (i) the bronchiolar cartilages (br). Minimal staining was noted in the skin (sk). A control sense probe showed no specific staining in murine tissues (data not shown).

FIG. 4

Expression and biochemical properties of the alternatively spliced isoforms of WTX.

(a) Cell lines from a mutation-bearing male and a healthy control express two WTX spliceforms to equivalent levels. (i) RT-PCR of cDNA from a primary cell line from case 04 and a healthy male control using primers flanking the alternatively spliced intron that amplify WTX_{S1} (1141 bp) and WTX_{S2} (310 bp). RNA negative controls represent mock cDNA (-RT) preparations to control for the presence of contaminating genomic DNA.

(b) Expression of wildtype WTX demonstrates two protein isoforms. Western blot of lysates of HEK293 cells transfected with full-length genomic construct expressing wild type FLAG-tagged WTX (lane 2). Lane 1: no transfection control.

(c) Proteins produced by both WTX protein isoforms bind β-catenin. A full-length genomic construct expressing FLAG-tagged WTX co-expressed with V5-tagged β-catenin in HEK293 cells. Immunoprecipitations were performed with anti-V5 antibody and detection with anti-FLAG.

(d) WTX protein isoforms show differential subcellular compartmentalisation. HEK293 cells transfected with either (i, ii) full length FLAG-tagged WTX_{S1} or (iii, iv) FLAG-tagged WTX_{S2} and immunostained with anti-FLAG antibody. Cells are counterstained with DAPI in (ii) and (iii). The protein produced by WTX_{S1} transcript stains the cytoplasm extensively in contrast to the isoform translated from WTX_{S2} which is almost exclusively nuclear in its localisation.

FIG. 5

Mutations associated with OSCS in males predict different combinations of WTX splice variants.

FIG. 6

Clinical phenotype of OSCS in females and males. (a-d) Radiographs of patient 21, a female with a de novo 1072C>T mutation in WTX (a) The skull base and calvarium is markedly thickened and sclerotic; (b-d) the metaphyses of the long bones show striations—longitudinal linear densities running parallel to the longitudinal axis of the bone shafts; (c) the diaphyses of the metacarpals and phalanges are under modelled. (e,g,i,j,k) Lethal phenotype in male individual 03 with a de novo mutation 1072C>T; and (f,h) individual 01 with the mutation 671 delC. Anomalies include omphalocele, patterning defects of the digits including duplication of the bony elements of digit II in both cases, and sclerosis and bowing of long bones. Cardiac defects (atrial and ventricular septal defects, pulmonary valve atresia and persistent ductus arteriosus) and genitourinary malformations (hydronephrosis) are also common accompaniments in these severely affected fetuses. (m-p) Phenotype of surviving males with mutations in WTX. (m) Axial CT scan of the skull demonstrating a thickened and sclerotic skull base and calvarium in case 05 also evident on the plain radiograph from case 04 (p); (n-o) linear striations are evident within the metaphyses of the long bones.

FIG. 7

Demonstration of the efficacy of two siRNAs (compared to scrambled controls) in knocking down the expression of WTX 1907 and 446 are two different siRNAs designed to target WTX and 1907C and 446C are scrambled controls. Knockdown is measured by RT-qPCR performed on cDNA prepared from cells transfected with these siRNAs. Untrans=untransfected control. siRNA 1907 targets both WTX_{S1} and WTX_{S2}, and produced 79% knockdown. siRNA 446 targets WTX_{S1} only and produced an 85% knockdown.

FIG. 8

Fraction of mutation-bearing X chromosomes subject to X-inactivation in females with OSCS. The proportion of cells that have the mutation-bearing X inactive is plotted as a function of the corresponding position of the causative mutation in the WTX protein. Phase was assigned in 7 familial instances and but unable to be ascertained in 10 sporadic cases. The latter group are graphically represented to favour exclusion of the null hypothesis that mutation bearing alleles are not preferentially inactivated. The values shown are the means±s.e.m for 3 separate assays; r² is calculated as the square of the Pearson product moment correlation coefficient.

FIG. 9

Multiple sequence alignment (Clustal W (1.83)) of eight full length wild type WTX polypeptide orthologues showing conserved and identical amino acid residues. Amino acid sequences included in the alignment are as follows: Mus musculus WTX (SEQ ID NO: 26); Rattus norvegicus WTX (SEQ ID NO: 40); Homo sapiens WTX (SEQ ID NO: 2); Pan troglodytes WTX (SEQ ID NO: 34), Canis familiaris WTX (SEQ ID NO: 36), Bos taurus WTX (SEQ ID NO: 38), Gallus gallus WTX (SEQ ID NO: 42) and Danio rerio WTX (SEQ ID NO: 44). Symbols used: “*” means that the residues or nucleotides in that column are identical in all sequences in the alignment; “:” means that conserved substitutions have been observed; “.” means that semi-conserved substitutions are observed. This multiple alignment shows contiguous amino acid regions that are identical regions shared by all aligned WTX polypeptide sequences.

FIG. 10

Expression and biochemical properties of the alternatively spliced isoforms of WTX. (a,b) Cell lines from a mutation-bearing male and a healthy control express two WTX spliceforms to equivalent levels. (a) RT-PCR of cDNA from a primary cell line from an affected male and a healthy male control using primers flanking the alternatively spliced intron that amplify WTX_{S1} (1141 bp) and WTX_{S2} (310 bp). RNA negative controls represent mock cDNA (-RT) preparations to control for the presence of contaminating genomic DNA. (b) qRT-PCR at four intragenic WTX loci indicating that both isoforms are expressed to similar levels in cDNA obtained from case 04 and healthy controls. Probes wtx_{—}2278897a1 and wtx_{—}227488897a2 lie within the intron spliced from WTX_{S2}; probes wtx6 and wtx9 are represented in both transcripts. Error bars represent s.e.m. An unpaired t-test (two-tailed) indicated no significant difference (P>0.5) between patient and controls; (c) Expression of wild type and mutation-bearing WTX alleles demonstrate two protein isoforms. Western blots of lysates of HEK293 cells transfected with a full-length genomic construct expressing wild type FLAG-tagged WTX, a construct expressing cloned WTX_{S2} and constructs containing mutations c.671delC (associated with lethality) and c.1506delA (associated with survival). (d) Proteins produced by both WTX protein isoforms bind β-catenin. A full-length genomic construct expressing FLAG-tagged WTX co-expressed with V5-tagged β-catenin in HEK293 cells. Immunoprecipitations were performed with anti-V5 antibody and detection with anti-FLAG. (e) WTX protein isoforms show differential subcellular localisation. Constructs expressing both FLAG-tagged WTX_{S1} and WTX_{S2} (i, ii) or WTX_{S2} alone (iii, iv). Expression of the longer isoform is associated with distribution around the plasma membrane. WTX_{S2} accumulates preferentially in the nucleus where it associates as punctuate aggregates.

FIG. 11

Cells transfected with a TOPFLASH Wnt-responsive promoter were treated with either siRNAs that had demonstrable ability to knockdown expression of WTX RNA (446 and 1907) or their cognate scrambled controls. These results were compared to the maximal amount of TOPFLASH activation achieved with a combination of the same siRNA in combination with the canonical Wnt signaling de-repressing agent LiCl. The ordinate expresses the degree of transactivation achieved by each siRNA alone relative to maximal stimulation with LiCl and siRNAs.

### DETAILED DESCRIPTION OF THE INVENTION

### Definitions

The terms “WTX polypeptide” and “WTX polynucleotide” as used herein include WTX sequences from any species, including but not limited to the polynucleotide and polypeptide sequences of human WTX (Wilms Tumour mutated on the X; FAM123B; AMER1) (SEQ ID NO: 1 and SEQ ID NO: 2, respectively), Mus musculus WTX (SEQ ID NO: 25 and SEQ ID NO: 26, respectively), Rattus norvegicus WTX (SEQ ID NO: 39 and SEQ ID NO: 40, respectively), Pan troglodytes WTX (SEQ ID NO: 33 and SEQ ID NO: 34, respectively), Canis familiaris WTX (SEQ ID NO: 35 and SEQ ID NO: 36 respectively), Bos taurus WTX (SEQ ID NO: 37 and SEQ ID NO: 38, respectively), Gallus gallus WTX (SEQ ID NO: 41 and SEQ ID NO: 42, respectively) and Danio rerio WTX (SEQ ID-NO: 43 and SEQ ID NO: 44, respectively).

Preferably a full length wild type WTX polypeptide refers to a polypeptide that comprises at least 1 or preferably at least 2 or preferably at least 3 or preferably at least 4 or preferably at least 5 or preferably at least 6 or preferably at least 7 or preferably at least 8 or preferably at least 9 or preferably at least 10 or preferably at least 11 or preferably at least 12, preferably at least 13, preferably at least 14, preferably at least 15, preferably at least 16, preferably at least 17, preferably at least 18, preferably at least 19, or preferably at least all of the following contiguous amino acid sequences, LPSFF, SIRRHRK, SLKSFDSLT, LKSFDSLTG LTGCDDIIA, SLTGCDDII, TYQGGGEEM, VTYQGGGEE, YQGGGEEMA, PNSDEGYYD, NSDEGYYDS EGYYDSTTP, GYYDSTTPG, DSYSGDALY, SYSGDALYE, LPRDSYSGD, PRDSYSGDA, METEEERL, FSQALV or RRSRSLD. Each of these contiguous regions of amino acid residues are identical between the aligned WTX polypeptides show in FIG. 8. As used herein the term WTX polypeptide also encompasses fragments and variants of a WTX polypeptide including but not limited to WTXS2 (SEQ ID NO: 4). Also encompassed within the term are truncated WTX polypeptides as described herein.

In one embodiment, a full length wild type WTX polypeptide or polynucleotide is the complete WTX (Wilms Tumour mutated on the X; FAM123B; AMER1) polypeptide or polynucleotide encoding the complete polypeptide. Preferably the full length wild type polypeptide is an 1135aa polypeptide as shown in SEQ ID NO: 2.

The term, “wild type” when used herein with reference to a WTX polynucleotide means a non-mutant form of a WTX polynucleotide. A mutant polynucleotide means a polynucleotide that has sustained a point mutation, insertion, deletion, substitution, amplification or translocation.

The term, “wild type” when used herein with reference to a WTX polypeptide means a non-mutant form of a WTX polypeptide. A wild type WTX polypeptide is a polypeptide that is capable of being from a wild type WTX polynucleotide.

The term “full length” as used herein with reference to a wild type polypeptide sequence means a polypeptide that comprises a contiguous sequence of amino acid residues where each amino acid residue has been expressed from each of its corresponding codons in the polynucleotide over the entire length of the coding region and resulting in a fully functional protein. As will be appreciated by a person of ordinary skill in the art, a “full length” polypeptide contains the amino acid sequence that corresponds to and has been expressed from each codon and every encoded by the polynucleotide comprising the entire coding region of the polypeptide, wherein each of said codons is located between the start codon and the termination codon normally associated with that coding region.

The term “antibody” refers to an immunoglobulin molecule having a specific structure that interacts (binds) specifically with a molecule comprising the antigen used for synthesizing the antibody or with an antigen closely related to it. An antibody binds selectively or specifically to a WTX polypeptide of the invention if the antibody binds preferentially to a WTX polypeptide, e.g. has less than 25%, preferably less than 10%, preferably less than 1% cross-reactivity with non-WTX polypeptides. Usually, the antibody will have a binding affinity (dissociation constant (Kd) value), for the antigen of no more than 10⁻⁷M, preferably less than about 10⁻⁸M, preferably less than about 10⁻⁹M. Binding affinity may be assessed using surface plasmon resonance.

Biological sample as used herein means any sample derived from a subject to be screened. The sample may be any sample known in the art in which WTX polynucleotide or polypeptide expression or activity can be detected. Included are any body fluids such as plasma, blood, saliva, interstitial fluid, serum, urine, synovial, cerebrospinal, lymph, seminal, amniotic, pericardial fluid and ascites. Also included are any cells such as stem cells, embryonic stem cells, pluripotent stem cells, mesenchymal stem cells, multipotent progenitor cells, osteoprogenitor cells, chondroblast cells; chondrocyte cells, adipocyte cells, osteoblast cells, osteoclast cells, osteocyte cells as well as tissues such as bone tissues but not limited thereto. Included are samples from any subjects such as normal healthy subjects with no clinical history of hyperostosis or OSCS and subjects with various bone-related disorders and conditions including hyperostosis, osteoporosis, OSCS, and subjects that have sustained bone injury, fracture, trauma, removal or breakage due to any means.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. Reference to nucleic acids, nucleic acid molecules, nucleotide sequences and polynucleotide sequences is to be similarly understood.

As used herein, a “double-stranded RNA” or “dsRNA” refers to a double stranded polynucleotide that includes, but is not limited to, a complex of ribonucleic acid molecules, having a duplex structure comprising two anti-parallel and substantially complementary nucleic acid strands. The duplex structure may be formed from different portions of one larger RNA molecule or from separate RNA molecules. The term, “RNAi molecule” where used herein is used interchangeably with “dsRNA” and should be considered to refer to the same double stranded polynucleotide as described above.

“siRNA” or “short interfering RNA” as used herein refers to a dsRNA that comprises two separate RNA strands as known and used in the art.

“shRNA” or “short hairpin RNA” as used herein refers to a dsRNA where the two strands are connected by an uninterrupted chain of nucleotides between the 3′-end of one strand and the 5′ end of the respective other strand to form a duplex structure.

The “antisense strand” of a dsRNA refers to the strand of a dsRNA that includes a region that is fully or substantially complementary to a target sequence.

The “sense strand” of a dsRNA refers to the strand of a dsRNA includes a region that is fully or substantially complementary to a region of the antisense strand. The “sense strand” may be fully or partially identical to the target sequence.

The term, a “nucleotide overhang” as used herein refers to the unpaired nucleotide or nucleotides that do not form part of a dsRNA duplex upon hybridization of anti-parallel strands. A nucleotide overhang includes extension of the 3′-end of one strand of the dsRNA beyond the 5′-end of the other strand, or vice versa.

“Blunt” or “blunt ended” dsRNA as used herein refers to a dsRNA that does not comprise nucleotide overhangs, i.e., is double-stranded over its entire length. Chemical caps or non-nucleotide chemical moieties conjugated to the 3′ end or 5′ end of an siRNA are not considered in determining complementarity of a dsRNA or in determining that a dsRNA comprises a nucleotide overhang or is blunt ended.

The term “antisense compounds” as used herein refers to all any oligomeric compounds that specifically hybridize to a target nucleic acid sequence and interfere with the normal function of the nucleic acid. Antisense compounds as used herein include, but are not limited to, “antisense nucleic acids” and “antisense oligonucleotides”. Antisense compounds may also be peptide nucleic acid compounds (PNA) or ribozymes.

The term “oligonucleotide” as used herein refers to an oligomer or polymer of ribonucleic acid (RNA) or deoxyribonucleic acid (DNA) and includes mimetics thereof. An oligonucleotide can consist of naturally-occurring nucleobases, sugars and internucleoside linkages but may also comprise non-naturally-occurring portions which perform similar functions. An antisense compound may comprise or consist of a “fragment” of a polynucleotide as defined herein

“Peptide nucleic acid” as used herein refers to a peptide nucleic acid (PNA) and includes PNAs that are “antisense compounds”, i.e., that hybridize to a target nucleic acid sequence. In a PNA oligonucleotide, the internucleoside linkages of the nucleotide residues have been replaced with an amide containing backbone, particularly an aminoethylglycine backbone and nucleobases are bound directly or indirectly to aza nitrogen atoms of the amide portion of the backbone.

“Ribozyme” as used herein refers to all types of ribozymes, including hammerhead, hairpin, a hepatitis δ virus, group I intron or RNaseP RNA (in association with an RNA guide sequence) or Neurospora VS RNA motifs. A ribozyme may be chemically synthesized or may be transcribed from a vector or multiple vectors, either in vitro or in vivo.

An “aptamer” as used herein refers to a nucleic acid ligand that binds to more than one site on a target molecule where binding is not “complementary,” i.e., is not due to base-pair formation between a nucleic acid ligand and a target nucleic acid sequence. An aptamer can be designed which binds to any envisionable target, including large and small biomolecules, such as lipids, carbohydrates and proteins and including proteins not known to bind nucleic acids as part of their biological function and nucleic acid-binding proteins. Aptamers can also be designed that bind other small molecules.

A “small molecule inhibitor” as used herein refers to a small molecule which binds to either a selected protein or a target RNA and inhibits the ability of the protein or RNA to perform its normal biological function or activity (e.g., reduces the rate or amount of protein binding to a particular target ligand or reduces the rate or amount of mRNA transcription from a coding sequence or polypeptide translation from an mRNA). Preferably the “small molecule inhibitor” is any small chemical moiety that inhibits WTX gene expression or activity. More preferably the small molecule inhibitor is a selective inhibitor of WTX gene expression or activity.

The term “small molecule” refers to a compound which has a molecular mass equal to or less than 5000 Daltons (5 kD), preferably less than 3 kD, still more preferably less than 2 kD, and most preferably less than 1 kD. In some cases it is highly preferred that a small molecule have a molecular mass equal to or less than 700 Da.

As used herein, an “inhibitor” refers to a compound that reduces, to a statistically significant extent, at least one activity of a reference compound. In the context of this invention, an inhibitor generally reduces an activity of an RNA, most commonly, but not limited to, reducing or eliminating the rate and/or extent of translation of an mRNA, reducing or eliminating processing of a pre-mRNA to produce a mature RNA; reducing or eliminating transport of an mRNA from nucleus to cytoplasm, or reducing or eliminating a regulatory function of an RNA molecule. Preferably the reduction in activity is sufficient to detect based on gross observation of cellular properties, e.g., growth rate, survival, morphology, and is preferably sufficient and of a nature to produce a therapeutic response in a patient in whom reduction in the expression of the target gene would be beneficial.

“Complementary”, as used herein describes a first nucleotide sequence in relation to a second nucleotide sequence and refers to the ability of a polynucleotide comprising the first nucleotide sequence to hybridize and form a duplex structure under certain conditions with a polynucleotide comprising the second nucleotide sequence. Such conditions can be, for example, moderately stringent conditions, stringent conditions or highly stringent conditions, as will be understood by a skilled person. Other conditions include, but are not limited to, any physiologically relevant conditions encountered inside an organism. The skilled person will be able to determine the specific appropriate conditions for testing the complementarity of two polynucleotide sequences as required for any particular hybridization application.

Complementary sequences, as used herein, may also include, or be formed entirely from, non-Watson-Crick base pairs and/or base pairs formed from non-natural and modified nucleotides, as long as the hybridization requirements outlined above are met.

“Fully complementary” as used herein, refers to base-pairing of a first nucleotide sequence to a second nucleotide sequence over the entire length of the first and second nucleotide sequences.

Where a first polynucleotide is referred to as “substantially complementary” with respect to a second polynucleotide sequence, the two sequences retain the ability to hybridize under certain conditions and can be fully complementary, or may faun one or more mismatched base pairs upon hybridization, but generally not more than 4, 3 or 2 mismatched base pairs upon hybridization.

The terms “complementary”, “fully complementary” and “substantially complementary” as used herein may refer, but are not limited to, base pairing between sense and antisense strands of a dsRNA, between the antisense strand of a dsRNA and a target sequence or between an antisense compound and a target sequence.

“Target sequence” as used herein refers to a contiguous portion of the nucleotide sequence of an mRNA molecule formed during the transcription of a WTX gene or fragment or variant thereof, including mRNA that is a product of RNA processing of a primary transcript.

The term “primer” refers to a short polynucleotide, usually having a free 3′OH group that is hybridized to a template and used for priming polymerization of a polynucleotide complementary to the target.

“Probe” as used herein refers to a short polynucleotide that is used to detect a polynucleotide sequence that is complementary to the probe, in a hybridization-based assay. The probe may consist of a “fragment” of a polynucleotide as defined herein.

A “fragment” of a polynucleotide sequence provided herein includes a subsequence of contiguous nucleotides that is capable of specific hybridization to a target of interest, e.g., a sequence that is at least 8 nucleotides in length. The fragments of the invention comprise or consist of 8, preferably 10, preferably 12, preferably 15, preferably 16, preferably 17, preferably 18, preferably 19, preferably 21, preferably 22, preferably 23, preferably 24, preferably 25, preferably 26, preferably 27, preferably 28, preferably 29, preferably 30, preferably 31, preferably 32, preferably 33, preferably 34, preferably 35, preferably 36, preferably 37, preferably 38, preferably 39, preferably 40, preferably 41, preferably 42, preferably 43, preferably 44, preferably 45, preferably 46, preferably 47, preferably 48, preferably 49, preferably 50, preferably 51, preferably 52, preferably 53, preferably 54, preferably 55, preferably 56, preferably 57, preferably 58, preferably 59, preferably 60, preferably 61, preferably 62, preferably 63, preferably 64, preferably 65, preferably 66, preferably 67, preferably 68, preferably 69, preferably 70, preferably 71, preferably 72, preferably 73, preferably 74, preferably 75, preferably 76, preferably 77, preferably 78, preferably 79, preferably 80, contiguous nucleotides of a specified polynucleotide sequence. A fragment of a polynucleotide sequence can be used as a primer, a probe, included in a microarray, or used in polynucleotide-based selection methods herein.

The term “polypeptide”, as used herein, encompasses amino acid chains of any length, but preferably at least 5 amino acid residues, preferably at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134 or preferably at least 1135 amino acid residues of a specified polypeptide sequence or any combination thereof, wherein the amino acid residues are linked by covalent peptide bonds. The term may refer to a polypeptide that is a purified natural product, or that has been produced partially or wholly using recombinant or synthetic techniques. The term may refer to an aggregate of a polypeptide such as a dimer or other multimer, a fusion polypeptide, a polypeptide fragment, a polypeptide variant, or derivative thereof.

A “fragment” of a polypeptide is a subsequence of the polypeptide that performs a function that is required for the biological activity or binding and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above signal peptide activity.

“Isolated” as used herein with reference to polynucleotide or polypeptide sequences describes a sequence that has been removed from its natural cellular environment. An isolated molecule may be obtained by any method or combination of methods as known and used in the art, including biochemical, recombinant, and synthetic techniques. The polynucleotide or polypeptide sequences may be prepared by at least one purification step.

“Isolated” when used herein in reference to a cell or host cell describes to a cell or host cell that has been obtained or removed from an organism or from its natural environment and is subsequently maintained in a laboratory environment as known in the art. The term is not limited to single cells, per se, but refers to a cell or host cell comprised in a cell culture and can include a single cell or single host cell.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

As used herein, the term “truncated” refers to a polynucleotide or polypeptide sequence that is different from the specifically identified corresponding wild type polynucleotide or polypeptide sequence wherein one or more nucleotides or amino acid residues of the polynucleotide or polypeptide that is “truncated” is absent or has been deleted. The deleted or absent nucleotides or amino acid residues can be contiguous residues of any number of residues. Truncated polypeptides or polynucleotides according to the invention can comprise missing or deleted contiguous residues from either the N- or C-terminus of the polypeptide, the 3′ or 5′ end of the polynucleotide or can comprise a deletion of an internal sequence of contiguous residues. An example of a truncated polypeptide of the invention can be, but is not limited to, WTXS2 (SEQ ID NO: 4), SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the polypeptides useful in the invention have biological activities that are the same or similar to those of the parent polypeptides or polynucleotides. The term “variant” with reference to polynucleotides and polypeptides encompasses all forms of polynucleotides and polypeptides as defined herein.

Variant polynucleotide sequences preferably exhibit at least 50%, at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 8 nucleotide positions, preferably at least 10 nucleotide positions, preferably at least 15 nucleotide positions, preferably at least 20 nucleotide positions, preferably at least 27 nucleotide positions, preferably at least 40 nucleotide positions, preferably at least 50 nucleotide positions, preferably at least 60 nucleotide positions, preferably at least 70 nucleotide positions, preferably at least 80 nucleotide positions and most preferably over the entire length of a polynucleotide of the invention.

Polynucleotide sequence identity may be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants also encompass those which exhibit a similarity to one or more of the specifically identified sequences that is likely to preserve the functional equivalence of those sequences and which could not reasonably be expected to have occurred by random chance. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻⁵, more preferably less than 1×10⁻⁶, more preferably less than 1×10⁻⁹, more preferably less than 1×10⁻¹², more preferably less than 1×10⁻¹⁵, more preferably less than 1×10⁻¹⁸ and most preferably less than 1×10⁻²¹ when compared with any one of the specifically identified sequences.

Polynucleotide sequence identity and similarity can also be determined in the following manner. The subject polynucleotide sequence is compared to a candidate polynucleotide sequence using sequence alignment algorithms and sequence similarity search tools such as in Genbank, EMBL, Swiss-PROT and other databases. Nucleic Acids Res 29:1-10 and 11-16, 2001 provides examples of online resources.

Use of BLASTN is preferred for use in the determination of sequence identity for polynucleotide variants according to the present invention.

BLASTN (from the BLAST suite of programs, version 2.2.13 Mar. 2007 in bl2seq (Tatiana A. et al, FEMS Microbiol Lett. 174:247-250 (1999), Altschul et al., Nuc. Acid Res 25:3389-3402, (1997)), is publicly available from NCBI (ftp://ftp.ncbi.nih.gov/blast/) or from NCB1 at Bethesda, Md., USA. The default parameters of bl2seq are utilized except that filtering of low complexity parts should be turned off.

The identity of polynucleotide sequences may be examined using the following UNIX command line parameters:

bl2seq-i nucleotideseq1-j nucleotideseq2-F F-p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Alternatively, variant polynucleotides hybridize to the specified polynucleotide sequence, or a complement thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing, incorporated herein by reference). Tm for polynucleotide molecules greater than about 100 bases can be calculated by the formula Tm=81.5+0.41% (G+C-log(Na+) (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for a polynucleotide of greater than 100 bases in length would be hybridization conditions such as prewashing in a solution of 6×SSC, 0.2%-SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

In one embodiment stringent conditions use 50% formamide, 5×SSC, 50 mM sodium phosphate (pH 6.8), 0.1% sodium pyrophosphate, 5×Denhardt's solution, sonicated salmon sperm DNA (50 pg/ml), 0.1% SDS, and 10% dextran sulphate at 42° C., with washes at 42° C. in 0.2×SSC and 50% formamide at 55° C., followed by a wash comprising of 0.1×SSC containing EDTA at 55° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

With respect to the DNA mimetics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the bl2seq program via the tblastx algorithm as described above.

The term “variant” with reference to polypeptides also encompasses naturally occurring, recombinantly and synthetically produced polypeptides. Variant polypeptide sequences preferably exhibit at least 50%, preferably at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 5 amino acid positions, preferably at least 7 amino acid positions, preferably at least 10 amino acid positions, preferably at least 15 amino acid positions, preferably at least 20 amino acid positions and most preferably over the entire length of a polypeptide used in the invention.

Polypeptide variants also encompass those which exhibit a similarity to one or more of the specifically identified sequences that is likely to preserve the functional equivalence of those sequences and which could not reasonably be expected to have occurred by random chance.

Polypeptide sequence identity and similarity can be determined in the following manner. The subject polypeptide sequence is compared to a candidate polypeptide sequence using BLASTP (from the BLAST suite of programs, version 2.2.14 [May 2006]) in bl2seq, which is publicly available from NCBI (ftp://ftp.ncbi.nih.gov/blast/). The default parameters of bl2seq are utilized except that filtering of low complexity regions should be turned off.

The similarity of polypeptide sequences may be examined using the following UNIX command line parameters:

bl2seq-i peptideseq1-j peptideseq2-F F-p blastp

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Variant polypeptide sequences preferably exhibit an E value of less than 1×10⁻⁵, more preferably less than 1×10⁻⁶, more preferably less than 1×10⁻⁹, more preferably less than 1×10⁻¹², more preferably less than 1×10⁻¹⁵, more preferably less than 1×10⁻¹⁸ and most preferably less than 1×10⁻²¹ when compared with any one of the specifically identified sequences.

Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Use of BLASTP as described above is preferred for use in the determination of polypeptide variants according to the present invention.

A variant WTX polypeptide includes a WTX polypeptide wherein the amino acid sequence differs from a WTX polypeptide herein by one or more conservative amino acid substitutions, deletions, additions or insertions which do not affect the biological activity of the peptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagines, glutamine; serine, threonine; lysine, arginine; and phenylalanine, tyrosine. Examples of conservative and semi-conservative substitutions can be taken from Table 1 below.

TABLE 1

| | Exemplary | Preferred |
|---|---|---|
| Original Residue | Substitutions | Substitution |
| Ala (A) | val; leu; ile; pro | val |
| Arg (R) | lys; gln; asn | lys |
| Asn (N) | gln; his; lys; arg | gln |
| Asp (D) | glu | glu |
| Cys (C) | ser | ser |
| Gln (Q) | asn; his | his |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | phe; val |
| | met; ala; phe | |
| Lys (K) | arg; gln; asn | arg |
| Met (M) | leu; phe; ile | leu |
| Phe (F) | leu; val; ile; ala; tyr | val |
| Pro (P) | ala | ala |
| Ser (S) | Thr; gly | gly |
| Thr (T) | ser; ala; pro | ser; ala |
| Trp (W) | tyr; phe | tyr |
| Tyr (Y) | trp; phe; thr; ser | phe |
| Val (V) | ile; leu; met; phe; ala; | leu |
| | norleucine | |

Naturally occurring residues are divided into groups based on common side-chain properties:

(1) hydrophobic: norleucine, met, ala, val, leu, ile;
(2) neutral hydrophilic: cys, ser, thr;
(3) acidic: asp, glu;
(4) basic: asn, gln, his, lys, arg:
(5) residues that influence chain orientation: gly, pro; and
(6) aromatic: trp, tyr, phe.

Non-conservative substitutions will entail exchanging a member of one of these classes for a member of another class.

Other variants include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g. D-amino acids or non-naturally occurring synthetic amino acids, e.g. beta or gamma amino acids and cyclic analogs.

Substitutions, deletions, additions or insertions may be made by mutagenesis methods known in the art. A skilled worker will be aware of methods for making phenotypically silent amino acid substitutions. See for example Bowie et al., 1990, Science 247, 1306.

A polypeptide as used herein can also refer to a polypeptide that has been modified during or after synthesis, for example, by biotinylation, benzylation, glycosylation, phosphorylation, amidation, by derivatization using blocking/protecting groups and the like. Such modifications may increase stability or activity of the polypeptide.

A “cell-penetrating peptide” as used herein refers to a peptide that forms part of a peptide-based delivery system that is capable of transporting an agent used according to the invention across cross biological membranes without the need of an additional carrier or via a cellular receptor. A cell-penetrating peptide can deliver an agent used in accordance with the invention to different cellular compartments, locations and subdomains and may also transport various types of agents.

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, including, but not limited to, mammalian cells such as Chinese hamster ovary cells (CHO) and non-mammalian cells such as yeasts or E. coli.

The term “expression cassette” refers to a genetic construct that includes the necessary elements that permit transcribing the insert polynucleotide molecule, and, optionally, translating the transcript into a polypeptide. An expression cassette typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence, is identified by the presence of a 5′ translation start codon and a 3′ translation stop codon. When inserted into a genetic construct or an expression cassette, a “coding sequence” is capable of being expressed when it is operably linked to promoter and terminator sequences and/or other regulatory elements.

“Operably-linked” means that the sequence to be expressed is placed under the control of regulatory elements.

“Regulatory elements” and “polynucleotide regulatory elements” mean any element that controls or influences the expression of a polynucleotide insert from a vector, genetic construct or expression cassette and includes promoters, transcription control sequences, translation control sequences, origins of replication, tissue-specific regulatory elements, temporal regulatory elements, enhancers, polyadenylation signals, repressors and terminators. Regulatory elements can be homologous or heterologous to the polynucleotide insert to be expressed from a vector, genetic construct or expression cassette according to the invention.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors. Promoters can be homologous or heterologous promoters and can be a constituitive promoter, an inducible promoter or a regulatable promoter.

“Homologous” as used herein with reference to the relationship between a polynucleotide regulatory element (PRE) and the sequence to which the PRE is operably linked in a genetic construct means that the PRE is normally associated in nature with the coding sequence to which it is operably linked in the construct. A homologous polynucleotide regulatory element may be operably linked to a polynucleotide of interest such that the polynucleotide of interest can be expressed from a, vector, genetic construct or expression cassette according to the invention.

“Heterologous” as used herein with reference to the relationship between a polynucleotide regulatory element (PRE) and the sequence to which the PRE is operably linked in a genetic construct means that the PRE is not normally associated in nature with the coding sequence to which it is operably linked in the construct. Such PREs may include promoters normally associated with different genes (other than WTX), and/or promoters isolated from any other bacterial, viral, eukaryotic, or mammalian cell.

The terms “to alter expression of” and “altered expression” of a polynucleotide or polypeptide of the invention, are intended to encompass the situation where genomic DNA corresponding to a polynucleotide of the invention is modified thus leading to altered expression of a polynucleotide or polypeptide of the invention. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

“Altered expression” in the context of the invention includes an increase or decrease in the levels of expression of a WTX polynucleotide transcript or mRNA that codes for a WTX polypeptide splice isoform. This term includes altered ratios of a WTX polynucleotide transcript or mRNA that codes for one WTX polypeptide splice isoform as compared to another, different, WTX polypeptide splice isoform. Such altered expression may be referred to as a bias in the ratio of splice isoforms or as an increased bias of one splice isoform.

The terms “modulate(s) expression”, “modulated expression” and “modulating expression” of a polynucleotide or polypeptide of the invention, are intended to encompass the situation where genomic DNA corresponding to a polynucleotide according to the invention is modified thus leading to modulated expression of a polynucleotide or polypeptide of the invention. Modification of the genomic DNA may be through genetic transformation or other methods known in the art for inducing mutations. The “modulated expression” can be related to an increase or decrease in the amount of messenger RNA and/or polypeptide produced and may also result in an increase or decrease in the activity of a polypeptide due to alterations in the sequence of a polynucleotide and polypeptide produced.

The terms “modulate(s) activity”, “modulated activity” and “modulating activity” of a polynucleotide or polypeptide of the invention, are intended to encompass the situation where genomic DNA corresponding to a polynucleotide according to the invention is modified thus leading to modulated expression of a polynucleotide or polypeptide of the invention. Modification of the genomic DNA may be through genetic transformation or other methods known in the art for inducing mutations. The “modulated activity” can be related to an increase or decrease in the amount of messenger RNA and/or polypeptide produced and may also result in an increase or decrease in the functional activity of a polypeptide due to alterations in the sequence of a polynucleotide and polypeptide produced.

As used herein, “modulates WTX gene expression or activity” or “WTX gene expression is modulated” and grammatical equivalents thereof refers to where any of: transcription of a WTX mRNA from a polynucleotide encoding a WTX amino acid sequence, translation of a WTX mRNA that encodes a WTX polypeptide amino acid sequence, functional activity of a WTX polypeptide in a cell or cell pathway is “modulated” or “altered.”

The term “WTX inhibitory agent” refers to any agent that inhibits the transcription or translation of a polynucleotide encoding a WTX polypeptide, and includes functional fragments and variants of a WTX polypeptide or polynucleotide. It also refers to any agent that inhibits the binding or activity of a WTX polypeptide.

“Transient” as used herein with reference to treatment refers to a period of time that is long enough to permit treatment of a bone-related disease or condition or limited to promoting bone growth, bone regeneration, bone healing or bone formation in a subject. Preferably, the “transient” treatment does not result in increased incidence of a detrimental effect in the subject. Preferably the detrimental effect that is avoided by “transient” treatment is cancer or tumorigenesis, but is not limited thereto. Preferably, the treatment is effected by the transient presence, at suitable levels, of an agent that modulates WTX gene expression or activity or of a WTX inhibitory agent, within the target cells of a subject.

“Subject” as used herein is preferably an animal. Preferably the animal is a mammal. Preferably the mammal includes human and non-human mammals such as cats, dogs, horses, cows, sheep, deer, mice, rats, primates (including gorillas, rhesus monkeys and chimpanzees), possums and other domestic farm or zoo animals, but not limited thereto. Preferably, the mammal is human.

The term “treat”, “treating” and “treatment” refer to therapeutic measures which alleviate, ameliorate, manage, prevent, restrain, stop or reverse progression of bone-related disease or conditions. The subject may show observable or measurable (statistically significant) increase in one or more of bone formation, bone healing, bone quality as known to those skilled in the art, as indicating improvement.

A “therapeutically effective amount” or “therapeutically effective dose” as used herein means an amount sufficient to produce the desired physiological effect or an amount capable of achieving the desired result, particularly for treating the desired disease or condition, including reducing or eliminating one or more symptoms or manifestations of the disease or condition.

As used herein, “bone-related disease or condition” refers to any condition that is the result of a bone abnormality or injury, due to any cause including, but not limited to a congenital defects, age-related defects, developmental defects and abnormalities, accidental injuries and surgical procedures.

Preferably the bone-related disease is osteoporosis including, but not limited to, primary osteoporosis, endocrine osteoporosis (hyperthyroidism, hyperparathyroidism, Cushing's syndrome, and acromegaly), hereditary and congenital forms of osteoporosis (osteogenesis imperfects, homocystinuria, Menkes' syndrome, and Rile-Day syndrome) and osteoporosis due to immobilization of extremities or as a side effect of drug treatment.

The term “promoting bone healing” as used herein refers to the healing of bone that has been treated according to the invention wherein the rate of healing is increased over that of a subject with the same disease or condition that has been treated as known in the art without reference to the invention described herein.

The term “promoting bone formation” as used herein refers to any bone growth or regeneration and in particular to increasing the regeneration and/or formation of bone so that the newly regenerated or formed bone is similar in strength, architecture and composition to normal healthy bone. Preferably, promoting bone formation is done in vitro or in vivo “Promoting bone formation” also includes where the bone formation is more rapid or in greater quantity than that observed in a subject treated for the same disease or condition as known in the art without reference to the invention described herein.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

WTX (Wilms Tumour mutated on the X; FAM123B; AMER1) is a cancer associated protein that is a component of the β-catenin destruction complex. WTX encodes a 1135aa protein that possesses a putative nuclear localization sequence, a β-catenin binding site located distal to residue Gly368⁴, and three binding sites for adenomatous polyposis coli (APC), another component of the β-catenin-sequestering complex^{10,14} (FIG. 2). The interaction of β-catenin with WTX in a complex with AXIN1, b-TrCP2, and APC promotes its proteasomal degradation⁴. WTX localizes to the plasma membrane in transfected cells via phosphatidylinositol (4,5)-bisphosphate binding site(s) localized to within residues 2-209¹⁴.

Recent work has shown that WTX protein forms a complex with β-catenin, AXIN1, b-TrCP2 (β-transducin repeat-containing protein 2) and APC which promotes β-catenin ubiquitination and degradation⁴. The degradation of β-catenin antagonizes Wnt/β-catenin signaling as degradation of β-catenin ultimately leads to reduced levels of this transcriptional coactivator for members of the TCF-LEF family of transcription factors in the nucleus (Logan et al. ANN Rev. Cell Dev. Biol 20: 781 (2004); Willert et al. Genes Dev. 20: 1394 (2006)). Mutations truncating translation of WTX N-terminal to Gly368, immediately prior to the AD (analogous to R358X; case 03, FIG. 2) enhance translocation of β-catenin to the nucleus⁴ and siRNA mediated knock-down of WTX increases transcription of Wnt-responsive reporter genes⁴.

In addition to this repressive action on Wnt signaling, WTX also regulates the distribution of APC between the microtubular cytoskeleton and the plasma membrane¹⁴. WTX possesses several domains that are conserved between its orthologues¹⁰ and its single human paralogue, FAM123A, which also possesses APC binding activity¹⁴ (FIG. 2).

Investigation of the cellular role of WTX has shown that this protein is a key component in the recruitment of APC to the plasma membrane and may contribute to tumorigenesis by abolishing functions of APC at the plasma membrane and activating those functions at microtubule ends¹⁴. WTX protein represses canonical Wnt signaling via its pro-degradative β-catenin binding activity. WTX protein has been shown to be somatically inactivated in 18-29% of cases of Wilms tumour. Additionally WTX exhibits tumor suppressor activity in an osteosarcoma cell line¹⁰.

Enhancement of Wnt signaling, either by overexpression of Wnt ligands⁵, deficiency of Wnt antagonists^{6,7}, activating mutations in Wnt receptors⁸ or stabilisation of β-catenin⁹ are associated with enhanced skeletal ossification in mice and humans. Mutations in PORCN, a human homologue of Drosophila Porcupine which also modulates Wnt signaling, lead to focal dermal hypoplasia^{11,12}, the phenotype of which includes multiple malformations and osteopathia striata.

Cellular mechanisms underlying the hyperostotic phenotypes caused by Wnt-related signaling defects have been variably attributed to osteoblast activation¹⁵, inhibition of osteoclast differentiation¹⁶ or re-direction of pluripotential stem cell differentiation from the adipocyte/chondrocyte lineages to anosteoblastic cell fate^{17,18} depending on the level within the pathway and/or developmental timing of activation of canonical Wnt signaling.

However, an effect of WTX on osteogenesis, either via Wnt signaling or by a different mechanism, has not been shown previously. Similarly, in vivo and in vitro effects of WTX polypeptide and fragments and variants thereof, on osteogenesis, osteoblast mineralization and/or in the growth and promotion of bone tissues and in promoting bone healing have not been demonstrated previously.

To the applicant's knowledge, no investigation to date has reported any effect of WTX protein with respect to bone health, bone formation and/or bone-related disease.

The applicants have now found that WTX polypeptide is a potential monogenic contributor to the skeletal and craniofacial phenotype observed in hyperostotic individuals.

The applicants of the present invention have found that germline mutations in WTX in males (n=5) and females (n=20) lead to the X-linked sclerosing bone dysplasia, osteopathia striata congenita with cranial sclerosis (OSCS; OMIM 1665001), which is characterised by increased bone density and craniofacial malformations. It is known that human WTX contains three APC binding domains, as shown in FIGS. 2 and 5, and exists as a number of variants and splice isoforms having varying effects on the development of OSCS in humans. The applicant's have also discovered that WTX protein is highly expressed in the fetal murine skeleton and that alternate splicing within the gene implicates subcellular localization and retention of an adenomatous polyposis coli binding domain (APCBD) and acidic domain (AD) as factors conferring survivability in males with OSCS.

In particular, the applicants have surprising and unexpectedly found that despite identical mutations occurring in the germline of individuals with OSCS and somatically in Wilms tumors, individuals with OSCS do not demonstrate a predisposition to tumor development (Example 4; Table 2). This observation is at odds with established understanding of tumor suppressor gene function and suggests the existence of temporal or spatial constraints on this action of WTX during tumorigenesis. A lack of tumor development in individuals with OSCS is supported by the applicant's comprehensive review of the literature on OSCS, in which no mention of a predisposition to cancer in OSCS patients could be located. In addition, the applicants could find no mention in the OSCS literature, of any involvement of WTX in effecting OSCS. See, for example, a review of OSCS by Behninger and Rott which details the clinical symptoms of family members with OSCS pedigrees, (Genetic Counseling 2000, Vol. 11(2): pp. 157-167). Behninger and Rott do not report cancer symptoms or an increase cancer predisposition in OSCS families, nor do they make any mention of WTX.

Based on these surprising findings, the applicants provide numerous aspects of an invention relating to a method of treatment for bone-related disease or conditions, including enhancing or promoting bone healing, bone growth, bone regeneration and bone formation via modulation of WTX gene expression and activity in bone cells.

### Proteins

Accordingly, the present invention provides numerous WTX polypeptide fragments and variants that effect cellular signaling via a WTX associated cell signaling pathway.

In a first aspect the present invention provides an isolated truncated “Wilms tumor gene on the X chromosome” (WTX) polypeptide
(a) from which at least five consecutive amino acids present in the full length wild type WTX polypeptide between amino acid residue 50 and amino acid residue 327 are absent, or
(b) consisting of a fragment of at least five consecutive amino acids present in amino acid residues 1 to 545 of the full length, wild type WTX polypeptide,

Preferably a truncated WTX polypeptide is capable of modulating the activity of WTX in a cell. Preferably a full length wild type polypeptide comprises the amino acid sequence of SEQ ID NO: 2

Preferably a full length wild type WTX polypeptide consists of an amino acid sequence of SEQ ID NO: 2.

Preferably a truncated WTX polypeptide comprises the contiguous amino acid sequence of SEQ ID NO: 4.

Preferably a full length WTX polypeptide comprises the amino acid sequence of SEQ ID NO: 24.

In one embodiment, a truncated WTX splice isoform lacks the activity of wild type WTX due to the absence of preferably at least 5 contiguous amino acids, preferably at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276 or 277 contiguous amino acids residues of the amino acid residues present between positions 50 and position 327 in a WTX polypeptide wherein the truncated WTX polypeptide modulates the expression or activity of a WTX associated signaling pathway in a subject as compared to the full length wild type WTX polypeptide.

In a preferred embodiment, the truncated WTX splice isoform lacks the activity of wild type WTX due to the absence of preferably at least 5 contiguous amino acids, preferably at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276 or 277 contiguous amino acids residues of the amino acid residues present between positions 50 and position 327 of the WTXS2 polypeptide (SEQ ID NO: 2) wherein the truncated WTX polypeptide modulates the expression or activity of a WTX associated signaling pathway in a subject as compared to the full length wild type WTX polypeptide.

The nucleotide sequence corresponding to WTXS2 is given in SEQ ID NO: 3. The nucleotide and amino acid sequences of WTXS2 are provided by the applicants for the first time. Both the nucleotide and amino acid sequence of WTXS2 form aspects of the invention.

In another embodiment, an isolated truncated WTX polypeptide consists of a fragment or variant of a WTX polypeptide that comprises preferably at least 5 or more consecutive amino acid residues, preferably at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, or 545 consecutive amino acid residues of the specified amino acid sequence, wherein the isolated truncated WTX polypeptide modulates the expression or activity of a WTX associated signaling pathway in a subject as compared to the wild type WTX polypeptide.

In another embodiment, an isolated truncated WTX polypeptide consists of a fragment or variant of a WTX polypeptide that comprises preferably at least 5 or more consecutive amino acid residues, preferably at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, or 545 consecutive amino acid residues of a WTX polypeptide consisting of SEQ ID NO: 24 and wherein the truncated WTX polypeptide modulates the expression or activity of a WTX associated signaling pathway in a subject as compared to the wild type WTX polypeptide.

In a particularly preferred embodiment, a truncated WTX polypeptide of the invention consists of the amino acid sequence of SEQ ID NO: 4, SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24 or an amino acid sequence at least 75% identical to the amino acid sequence the amino acid sequence of SEQ ID NO: 4, SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24.

In a further embodiment, a truncated WTX polypeptide of the invention consists of the amino acid sequence of SEQ ID NO: 4, SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24, or an amino acid sequence at least 75% identical to the amino acid sequence the amino acid sequence of SEQ ID NO: 4, SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24 wherein the truncated WTX polypeptide acts as a competitive inhibitor of wild type WTX, causing an increase in WTX associated signaling that biases osteoprogenitor cell differentiation, resulting in an increase in osteoblastogenesis, a reduction in chondrocyte differentiation and/or stimulated or enhanced mineralization in cells, particularly bone cells.

In another embodiment, a truncated WTX polypeptide of the invention that consists of the amino acid sequence of SEQ ID NO: 4, SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24 and fragments or variants thereof as described herein wherein the truncated WTX polypeptide blocks recruitment of the β-catenin degradation complex to the plasma membrane by inhibiting full length wild type WTX binding at the plasma membrane.

Isolated truncated WTX polypeptide fragments and variants in accordance with the invention may be identified and analyzed using a variety of computer based methods as known in the art and as described previously herein.

Multiple sequence alignments of a group of related sequences can be carried out with CLUSTALW (Thompson, et al., Nucleic Acids Research, 22:4673-4680 (1994), http://www-igbmc.u-strasbg.fr/BioInfo/ClustalW/Top.html) or T-COFFEE (Cedric Notredame et al., J. Mol. Biol. 302: 205-217 (2000))) or PILEUP, which uses progressive, pairwise alignments. (Feng et al., J. Mol. Evol. 25, 351 (1987)).

In a particular embodiment, a truncated WTX polypeptide of the invention comprises at least a β-catenin binding domain. In another embodiment, a truncated WTX polypeptide of the invention comprises at least a plasma membrane localization domain. In yet another embodiment, a truncated WTX polypeptide of the invention comprises at least a nuclear localization domain. These protein domains and other protein domains and motifs are readily identified by the person of skill in the art and as described herein.

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch et al., Nucleic Acids Res. 22, 3583 (1994); Hofmann et al., Nucleic Acids Res. 27, 215 (1999)) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., Nucleic Acids Res. 30, 235 (2002)). Prosearch is a tool that can search SWISS-PROT and EMBL databases with a given sequence pattern or signature.

Proteins can be classified according to their sequence relatedness to other proteins in the same genome (paralogues) or a different genome (orthologues). Orthologous genes are genes that evolved by speciation from a common ancestral gene and normally retain the same function as they evolve. Paralogous genes are genes that are duplicated within a genome and genes may acquire new specificities or modified functions which may be related to the original one. Phylogenetic analysis methods are reviewed in Tatusov et al., Science 278, 631-637, 1997).

In addition to the computer/database methods described above, polypeptide variants may be identified by physical methods, for example by screening expression libraries using antibodies raised against polypeptides of the invention (Sambrook et al., Molecular Cloning: A Laboratory Manual, 2nd Ed. Cold Spring Harbor Press, 1987) by recombinant DNA techniques also described by Sambrook et al. or by identifying polypeptides from natural sources with the aid of such antibodies.

Polypeptides, including variant polypeptides, may be prepared using peptide synthesis methods well known in the art such as direct peptide synthesis using solid phase techniques (e.g. Merrifield, 1963, in J. Am. Chem. Soc. 85, 2149; Stewart et al., 1969, in Solid-Phase Peptide Synthesis, WH Freeman Co, San Francisco Calif.; Matteucci et al. J. Am. Chem. Soc. 103:3185-3191, 1981) or automated synthesis, for example using a Synthesizer from Applied Biosystems (California, USA). Mutated forms of the polypeptides may also be produced using synthetic methods such as site-specific mutagenesis of the DNA encoding the amino acid sequence as described by Adelmen et al; DNA 2, 183 (1983).

The polypeptides and variant polypeptides herein are preferably isolated. They may be isolated or purified from natural sources using a variety of techniques that are well known in the art (e.g. Deutscher, 1990, Ed, Methods in Enzymology, Vol. 182, Guide to Protein Purification,). Technologies include HPLC, ion-exchange chromatography, and immunochromatography but are not limited thereto.

Alternatively the polypeptides and variant polypeptides may be expressed recombinantly in suitable host cells and separated from the cells as discussed below. The polypeptides and variants have utility in generating antibodies, and generating ligands amongst other uses.

An isolated truncated WTX polypeptide of the invention can also be modified and expressed as a fusion protein comprising a truncated WTX polypeptide of the invention.

WTX fusion proteins are proteins formed by the fusion of at least one molecule of WTX (or a fragment or variant thereof) to at least one molecule of a different protein of interest. The protein of interest can be, by way of non-limiting example, a therapeutic protein (or fragment or variant thereof) or a detectable protein used in an assay to detect cell or tissue specific expression of a WTX protein. A WTX fusion protein in accordance with the invention comprises at least a fragment or variant of a WTX protein and at least a fragment or variant of a different protein of interest, which are associated with one another, preferably by genetic fusion (i.e., a WTX fusion protein is generated by translation of a nucleic acid in which a polynucleotide encoding all or a portion of a different protein of interest is joined in-frame with a polynucleotide encoding all or a portion of a truncated WTX polynucleotide). A WTX protein and the different protein of interest, once part of a WTX fusion protein, may each be referred to herein as a “portion”, “region” or “moiety” of a WTX fusion protein (e.g., a “WTX protein portion”).

A “different protein of interest” can be fused to a truncated WTX polypeptide of the invention for the purposes of facilitating WTX polypeptide detection, targeting, purification, isolation, degradation, immobilization or folding. A WTX polypeptide fusion may function to increase the levels of expression, concentration, solubility, transport, or stability of a WTX polypeptide.

Additional modifications of a truncated WTX polypeptide of the invention may include glycosylation, GPI anchor formation, or myristoylation or any other protein modification as known and used in the art.

### Polynucleotides

The invention also provides an isolated polynucleotide that is capable of expressing a truncated WTX polypeptide of the invention or is a nucleotide sequence that is at least 75% identical to the nucleotide sequence of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23.

In a particular embodiment, the invention provides an isolated polynucleotide of the invention which consists of the nucleotide sequence of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23.

The nucleic acid molecules of the invention or otherwise described herein are preferably isolated. They can be isolated from a biological sample using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser. The nucleic acid molecules of the invention can be amplified using primers, as defined herein, derived from the polynucleotide sequences of the invention.

Further methods for isolating polynucleotides include use of all, or portions of, a polynucleotide of the invention, preferably a polynucleotide having the sequence set forth in SEQ ID NO: 1 or SEQ ID NO: 25 as hybridization probes. The technique of hybridizing labeled polynucleotide probes to polynucleotides immobilized on solid supports such as nitrocellulose filters or nylon membranes, can be used to screen genomic or cDNA libraries. Similarly, probes may be coupled to beads and hybridized to the target sequence. Isolation can be effected using known art protocols such as magnetic separation. Exemplary stringent hybridization and wash conditions are as given above.

Polynucleotide fragments may be produced by techniques well-known in the art such as restriction endonuclease digestion and oligonucleotide synthesis.

A partial polynucleotide sequence may be used as a probe, in methods well-known in the art to identify the corresponding full length polynucleotide sequence in a sample. Such methods include PCR-based methods, 5′RACE (Methods Enzymol. 218: 340-56 (1993); Sambrook et al., Supra) and hybridization-based method, computer/database-based methods. Detectable labels such as radioisotopes, fluorescent, chemiluminescent and bioluminescent labels may be used to facilitate detection. Inverse PCR also permits acquisition of unknown sequences, flanking the polynucleotide sequences disclosed herein, starting with primers based on a known region (Triglia et al., Nucleic Acids Res 16, 8186, (1998)). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Supra). Primers and primer pairs which allow amplification of polynucleotides of the invention, also form a further aspect of this invention.

Variants (including orthologues) may be identified by the methods described. Variant polynucleotides may be identified using PCR-based methods (Mullis et al., Eds. 1994 The Polymerase Chain Reaction, Birkhauser). Typically, the polynucleotide sequence of a primer, useful to amplify variants of polynucleotide molecules by PCR, may be based on a sequence encoding a conserved region of the corresponding amino acid sequence.

Further methods for identifying variant polynucleotides include use of all, or portions of, the specified polynucleotides as hybridization probes to screen genomic or cDNA libraries as described above. Typically probes based on a sequence encoding a conserved region of the corresponding amino acid sequence may be used. Hybridization conditions may also be less stringent than those used when screening for sequences identical to the probe.

The invention also provides an expression cassette comprising,
(a) a polynucleotide of the invention
(b) fragments of 8 to 80 contiguous nucleotides of the polynucleotide sequence in (a), or
(c) a polynucleotide complementary to the polynucleotide sequence in (a) or (b), wherein the polynucleotide in any of (a) to (c) is under the control of a promoter.

The expression cassettes described herein may comprise one or more of the disclosed polynucleotide sequences and/or polynucleotides encoding the disclosed polypeptides, of the invention. The expression cassette can be any expression cassette that is appropriate to drive expression of a truncated WTX polynucleotide in cells, either in vitro or in vivo. In one embodiment, the expression cassette comprises a polynucleotide of the invention operatively linked to 5′ or 3′ untranslated regulatory sequences. The design of a particular expression cassette will depend on various factors including the host cells in which the operatively linked polynucleotide is to be expressed and the desired level of polynucleotide expression.

Likewise, the selection of various promoters, enhancers, selectable markers, or other elements for an expression vector will depend on various factors including the host cells and expression levels discussed above. In one embodiment, the expression cassette comprises a homologous promoter operatively linked to a truncated WTX polynucleotide of the invention. In another embodiment, the expression cassette comprises a heterologous promoter operatively linked to a truncated WTX polynucleotide of the invention. In one embodiment, the homologous or heterologous promoter is an inducible, repressible or regulatable promoter. A suitable promoter may be chosen and used under the appropriate conditions to direct high-level expression of a truncated WTX polynucleotide of the invention. Many such elements are described in the literature and are available through commercial suppliers.

By way of example only, promoters useful in the expression cassettes can be any suitable eukaryotic or prokaryotic promoter. In one embodiment, the eukaryotic promoter can be a eukaryotic RNA polymerase I (pol I), RNA polymerase II (pol II), or RNA polymerase III (pol III). Expression levels of an operably linked polynucleotide in a particular cell type will be determined by the nearby presence (or absence) of specific gene regulatory sequences (e.g., enhancers, silencers and the like). Any suitable promoter/enhancer combination (see: Eukaryotic Promoter Data Base EPDB) can be used to drive expression of a truncated WTX polynucleotide of the invention.

Additional promoters useful in expression cassettes include β-lactamase, alkaline phosphatase, tryptophan, and tac promoter systems which are all well known in the art. Yeast promoters include 3-phosphoglycerate kinase, enolase, hexokinase, pyruvate decarboxylase, glucokinase, and glyceraldehydrate-3-phosphanate dehydrogenase but are not limited thereto.

Prokaryotic promoters useful in expression cassettes include constitutive promoters as known in the art (such as the int promoter of bacteriophage lamda and the bla promoter of the beta-lactamase gene sequence of pBR322) and regulatable promoters (such as lacZ, recA and gal). A ribosome binding site upstream of the coding sequence may also be required for expression.

Enhancers useful in the expression cassettes include SV40 enhancer, cytomegalovirus early promoter enhancer, globin, albumin, insulin and the like.

In one embodiment, the expression cassette may be driven by a T3, T7 or SP6 cytoplasmic expression system.

The choice of a particular promoter/enhancer/cell type combination for protein expression is within the ordinary skill of those in the art of molecular biology (see, for example, Sambrook et al. (1989) which is incorporated herein by reference).

The expression cassette may conveniently include a selection gene or selectable marker. Typically an antibiotic resistance marker such as ampicillin, methotrexate, or tetracycline is used.

An expression cassette containing a polynucleotide of the invention can be incorporated into any suitable vector capable of expressing that polynucleotide or, where applicable, an encoded polypeptide of the invention in vitro or in a host cell. Examples of suitable vectors include, but not limited to, plasmid DNA vectors, viral DNA vectors (such as adenovirus and adeno-associated virus), or viral RNA vectors (such as a retroviral vectors). Such vectors are also referred to herein as “plasmid vectors” or “viral vectors” but may also be referred to herein as “recombinant plasmids” or “recombinant viral vectors.”

Included are plasmid and phage vectors such as, pUC18, pU19, Mp18, Mp19, ColE1, PCR1 and pKRC; lambda gt10 and M13 plasmids such as pBR322, pACYC184, pT127, RP4, p1J101, SV40 and BPV. Also included are vectors such as, but limited to, cosmids, YACS, BACs shuttle vectors such as pSA3, PAT28 transposons (such as described in U.S. Pat. No. 5,792,294) and the like.

Suitable viral vectors include, but are not limited to vectors derived from adenovirus (AV); adeno-associated virus (AAV); retroviruses (e.g., lentiviruses (LV), Rhabdoviruses, murine leukemia virus); herpes virus, and the like. Viral vectors employed herein can be appropriately modified by pseudotyping with envelope proteins or other surface antigens from other viruses, or by substituting different viral capsid proteins, as known and used in the art.

Selection of suitable viral vectors, methods for inserting a polynucleotide of the invention into the vector, and methods of delivering the viral vector to a particular cell type, including in vivo, are within the skill in the art. See, for example, Domburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference.

In a preferred embodiment, the viral vector is derived from AV or AAV. In a particularly preferred embodiment, the viral vector is a recombinant AAV vector comprising, for example, a U6 or H1 RNA promoters, or the cytomegalovirus (CMV) promoter.

AAV vectors suitable for use in the invention, including methods of constructing the recombinant vector and of delivery are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

A plasmid or viral vector as described herein may be used to drive transient expression of a polynucleotide of the invention, in vivo, according to the methods of the invention. Such vectors can be administered repeatedly as necessary. Such a vector can be termed a gene therapy vector. Delivery of a gene therapy vector to a subject can be, for example, by systemic delivery, such as by intravenous or intramuscular injection. Delivery of a gene therapy vector can also be, for example, by direct application, local administration (see U.S. Pat. No. 5,328,470) or by stereotactic injection (see e.g., Chen et al. (1994) Proc. Natl. Acad. Sci. USA 9 1:3054 3057). A pharmaceutical preparation of the gene therapy vector can include the gene therapy vector in an acceptable diluent or excipient, or can comprise the vector in a slow release matrix. As used herein a “slow release matrix” refers to any composition used for delivery that reduces the rate of the gene therapy vector into a subject relative to the rate of the gene therapy vector when not delivered in a slow release matrix. A slow release matrix can include, but is not limited to, a gel, a slurry, a paste, an artificial tissue construct, a polymeric matrix, a non-polymeric matrix, an porous implant, a non-porous implant, a capsule, a particle, a microparticle, a nanoparticle, a suppository or any other slow release matrix as is known or used in the art.

A gene therapy vector can be administered to a target cell (the term “host cell” is also used herein in this context) in vivo or that has been ex-planted from a subject. The transformed, ex-planted target cell can then be reintroduced into the subject by any suitable means that allows for introduction into a desired target region of the subject.

Successful introduction of the vector into a target or host cell can be monitored using various methods as known and used in the art. Transient transfection, for example, can be monitored using a reporter gene construct that expresses a fluorescent marker, such as Green Fluorescent Protein (GFP). Any other suitable marker as known in the art may also be employed in the gene therapy vector described herein.

In one embodiment, a polynucleotide of the invention may be inserted into a gene therapy vector and used in a human patient to overexpress a polypeptide of the invention.

In one embodiment, the gene therapy vector can be produced intact from transformed or transduced cells, e.g., retroviral vectors, isolated and then used to transduce additional target or host cells.

Methods for producing and using expression cassettes and vectors are well known in the art and are described generally in Sambrook et al., (supra), and Ausubel et al., Current Protocols in Molecular Biology, Greene Publishing, 1987. Methods for transforming selected host cells with the vectors are also known, for example, the calcium chloride treatment described by Cohen, S N; PNAS 69, 2110, 1972.

The invention also provides an isolated host cell comprising an expression cassette or vector of the invention. In another embodiment the invention provides a cell culture comprising the isolated host cell of the invention.

Various host cells can comprise an expression cassette or vector as described herein. Such host cells may be derived from prokaryotic or eukaryotic sources, for example yeast, bacteria, fungi, insect (e.g. baculovirus), animal, mammalian or plant organisms. In one embodiment the host cells are isolated host cells. Prokaryotes most commonly employed as host cells are strains of E. coli. Other prokaryotic hosts include Pseudomonas, Bacillus, Serratia, Klebsiella, Streptomyces, Listeria, Saccharomyces, Salmonella and Mycobacteria but are not limited thereto.

Eukaryotic cells for expression of recombinant protein include but are not limited to Vero cells, HeLa, CHO (Chinese Hamster ovary cells), 293, BHI cells, MDCK cells, and COS cells as well as prostate cancer cell lines such as PrEC, LNCaP, Du 145 and RWPE-2. The cells are available from ATCC, Virginia, USA.

In one embodiment the host cell is a mammalian cell that is removed or ex-planted from a subject, including but not limited to a pluripotent stem cell, a multipotent progenitor cell, a mesenchymal stem cell, an osteoprogenitor cell, an osteoblast, an osteoclast, an osteocyte, a chondroblast, a chondrocyte or an adipocyte. In one embodiment the host cell is an MC3T3 cell.

In one embodiment, the host cell can obtained from a subject, transformed with a recombinant plasmid or recombinant viral vector, that can be a gene therapy vector as described herein, and then replaced in the subject to provide a transient gene therapeutic effect or a transient gene therapy treatment in the subject. Replacement may be by any means, including but not limited to, direct application, replacement in a cell or non-cell scaffold or matrix, infusion or injection.

In yet another embodiment, the invention provides a non-human transgenic animal comprising an expression cassette, vector or host cell of the invention. The non-human transgenic animal can be any non-human animal including, but not limited to, a non-human primate, a cow, a pig, a chicken, a sheep, a rat, a mouse, a zebrafish or a fruit fly

Another aspect of the invention provides a method of making a polypeptide of the invention comprising the steps of transforming a host cell with an expression cassette or vector of the invention, culturing the host cell under conditions whereby the polypeptide is expressed and subsequently isolating the expressed polypeptide. In a particular embodiment, the host cell transformed in the method of making the polypeptide of the invention is comprised in a non-human transgenic animal.

The presence of a polynucleotide or polypeptide of the invention their level of expression in a sample, cell or cell culture may be determined according to methods known in the art such as Southern Blotting, Northern Blotting, FISH or quantitative PCR to quantify the transcription of mRNA [(Thomas, Pro. NAH, Acad. Sci. USA 77: 5201-5205 1980), (Jain K K, Med Device Technol. 2004 May; 15(4):14-7)], dot blotting, (DNA analysis) or in situ hybridization using an appropriately labeled probe, based on the marker sequences provided herein.

Host cells comprising expression cassettes and vectors according to the invention are useful in methods for recombinant production of polypeptides. Such methods are well known in the art (see for example Sambrook et al. supra). The methods commonly involve the culture of host cells in an appropriate medium in conditions suitable for or conducive to, expression and selection of a polypeptide of the invention. Cells with a selectable marker may additionally be grown on medium appropriate for selection of host cells expressing a polypeptide of the invention. Transformed host cells expressing a polypeptide of the invention are selected and cultured under conditions suitable for expression of the polypeptide. The expressed recombinant polypeptide, may be separated and purified from the culture medium using methods well known in the art including ammonium sulfate precipitation, ion exchange chromatography, gel filtration, affinity chromatography, electrophoresis and the like (e.g. Deutscher, Ed, 1990, Methods in Enzymology, Vol 182, Guide to Protein Purification). Host cells may also be useful in methods for production of a product generated by an expressed polypeptide of the invention.

The invention also contemplates use or detection of the truncated WTX polypeptides and polynucleotides of the invention as diagnostic or prognostic markers. For example, use as markers for bone associated diseases and conditions, such as but not limited to those diseases and conditions discussed herein is contemplated. Methods for detection and use of such markers are well-known to those skilled in the art, and are described for example in U.S. Pat. No. 7,355,025.

In one aspect, the invention provides a method of treating a bone-related disease or condition in a subject, the method comprising administration to a subject in need thereof, of a therapeutically effective amount of an agent that modulates the expression or activity of a WTX polypeptide in the subject.

Bone-related disease or conditions contemplated for treatment within the context of the present invention include, but are not limited to, osteopenia, osteoporosis (including drug induced and/or iatrogenic osteoporosis), osteoarthritis, rheumatoid arthritis, periprosthetic bone loss, osteolysis, metastatic bone disease, and Paget's disease or any other bone trauma that requires increased or stimulated bone healing or bone formation.

In another aspect, the invention provides a method for treating a bone-related disease or condition in a subject, the method comprising administration to a subject in need thereof, of a therapeutically effective amount of an agent that modulates the expression or activity of a WTX associated signaling pathway in the subject.

Preferably the bone related-disease that is treated according to the methods of the invention is any form of osteoporosis, including but not limited to generalized osteoporosis, idiopathic osteoporosis, drug-induced osteoporosis, primary osteoporosis, endocrine osteoporosis (hyperthyroidism, hyperparathyroidism, Cushing's syndrome, and acromegaly), hereditary and congenital forms of osteoporosis (osteogenesis imperfects, homocystinuria, Menkes' syndrome, and Rile-Day syndrome) and osteoporosis due to immobilization of extremities.

A WTX associated signaling pathway refers to any cell signaling pathway that is modulated in response to an alteration in WTX polynucleotide expression or activity.

In a further aspect, the invention provides a method of promoting bone healing or bone formation, the method comprising contacting cells with an agent that modulates the expression or activity of a WTX polypeptide.

Promotion of bone healing or bone formation may be required to effect a treatment of a bone condition that is the result of any bone-related trauma including, but not limited to, bone fracture, rupture, break or breakage resulting from any cause including accidental fracture, rupture, break or breakage or as a result of a medical or surgical procedure.

In one embodiment the surgical procedure is a bone graft, a bone re-alignment, a bone straightening procedure, a bone lengthening procedure, a bone shortening procedure, a bone replacement procedure, a joint replacement procedure, a bone repair procedure, a bone reconstruction procedure or any other surgical procedure where successful recovery requires bone growth, bone regeneration, bone healing or bone formation.

In one embodiment the surgical procedure is a reconstruction. In another embodiment the surgical procedure is a bone repair using a metal pin, rod or fiber, an artificial joint replacement or a dental implant.

In another aspect, the invention provides a method of modulating osteoprogenitor cell differentiation, the method comprising contacting osteoprogenitor cells with an agent that modulates the expression or activity of a WTX polypeptide.

In yet another aspect, the invention provides a method for increasing or stimulating osteoblast differentiation, the method comprising administration to multipotent progenitor cells of an agent that modulates the expression or activity of a WTX polypeptide.

Progenitor cells are comparable to adult stem cells but are said to be in a farther stage of cell differentiation. Progenitor cells are may be unipotent or multipotent. The potency of a progenitor cell will depend on the parent stem cell from which the progenitor cell is derived and on their particular biological role or “niche.” Progenitor cells may be formed and transported in a colony, with the right conditions for them to grow and differentiate into their target tissues or they may migrate through the body and localize in different tissues.

An osteoprogenitor cell as used herein is a type of bone cell consisting of undifferentiated stromal cells from which the osteoblasts are derived. Osteoprogenitor cells may also be characterized as mesenchymal stem cells that differentiate into osteoblasts and are also known in the art as preosteoblast cells. Osteoprogenitor cells are located in the periosteum and the bone marrow.

Mesenchymal stem cells (MSC) as used herein are osteochondrogenic (or osteogenic, chondrogenic, osteoprogenitor, etc.) cells. In vitro differentiation of a single MSC can lead to chondrocytes or osteoblasts, depending on the particular conditions used. In vivo, differentiation of an MSC in a vascularized area (such as bone) can yield an osteoblast, whereas differentiation of an MSC in a non-vascularized area (such as cartilage) can yield a chondrocyte. Alternatively factors that determine cell fates are humoral such as cytokines, hormones and growth factors.

Osteoblast cells are mononucleate cells responsible for bone formation. Osteoblasts produce osteoid (which consists mainly of Type I collagen) and are responsible for mineralization of the osteoid matrix. Osteoblasts build and reshape bone tissue, as contrasted with osteoclasts, which resorb bone tissue.

A chondroblast is a cell which originates from a mesenchymal stem cell and forms chondrocytes. Chondroblasts that become embedded in a cartilage matrix are called chondrocytes. Chondrocytes are the cells found in cartilage. Chondrocytes produce and maintain the cartilaginous matrix, which consists mainly of collagen and proteoglycans.

In a further aspect, the invention provides a method for stimulating or enhancing mineralization of matrix by cells, the method comprising administration to cells of an agent that modulates the expression or activity of a WTX polypeptide.

As used herein, mineralization refers to the process where a substance is converted from an organic substance to an inorganic substance, thereby becoming mineralized.

Mineralization is a normal biological process which takes place during the life of an organism and includes, for example, but not limited thereto, the formation of bone tissue or egg shells, largely with calcium.

In one embodiment, the agent used in the methods of the invention is any agent that inhibits the expression or activity of a WTX polypeptide.

Preferably the agent used in the methods of the invention is selected from the group consisting of:
(i) an antibody capable of specifically binding to a WTX polypeptide,
(ii) an antisense compound that inhibits the expression of said WTX polypeptide,
(iii) an expression vector encoding said antisense compound,
(iv) a dsRNA that inhibits the expression of said WTX polypeptide,
(v) one or more expression vectors encoding said dsRNA,
(vi) a ribozyme that inhibits the expression of said WTX polypeptide,
(vii) one or more expression vectors encoding said ribozyme,
(viii) a fragment of a WTX polypeptide,
(ix) a truncated WTX polypeptide of the invention,
(x) an expression vector comprising a polynucleotide which encodes a polypeptide of the invention,
(xi) a fusion protein comprising a truncated WTX polypeptide of the invention,
(xii) a small molecule inhibitor of WTX gene expression or activity,
(xiii) an aptamer that inhibits WTX gene expression or activity, and
(xiv) a peptide nucleic acid that inhibits WTX gene expression or activity.

In one embodiment, the agent useful in the methods of the invention inhibits the expression or activity of substantially all WTX polypeptide isoforms that would be expressed or active in a particular cell, tissue-type or tissue.

Preferably the useful agent inhibits the expression or activity of all WTX polypeptide isoforms that would be expressed or active in a particular cell, tissue-type or tissue.

In another embodiment, the useful agent increases the ratio of a splice isoform of WTX polypeptide relative to a full length wild type WTX polypeptide.

Preferably, the increase in ratio of one WTX splice isoform polypeptide relative to a full length wild type WTX polypeptide is the result of the selective increase in the transcription of one mRNA as compared to a variant or alternative mRNA transcribed from the same gene can be achieved as known in the art. For example, U.S. Pat. No. 6,686,148 (which is incorporated herein by reference) describes methods for achieving such a bias in RNA transcription between variants by selecting for small molecule inhibitors. Additional agents useful in the inventive methods may also be employed to achieve this effect, including but not limited to, antibodies, dsRNAs, s antisense compounds, ribozymes, aptamers, WTX polypeptide variants and truncated WTX polypeptides, each of which as known in the art or described herein.

Preferably the splice isoform is a shorter splice isoform. Preferably a shorter splice isoform comprises a contiguous sequence of amino acid residues that are fewer contiguous amino acid residues than found in a full length wild type WTX polypeptide due to a deletion of any number of contiguous amino acid residues from either the C-terminal end, N-terminal end or internally, of a WTX polypeptide.

Preferably a shorter splice isoform of a WTX polypeptide comprises an isolated truncated WTX polypeptide from which at least five consecutive amino acids present in the full length wild type WTX polypeptide between amino acid residue 50 and amino acid residue 327 are absent.

Preferably a shorter splice isoform of a WTX polypeptide is the WTXS2 polypeptide as described herein. More preferably the WTXS2 polypeptide consists of SEQ ID NO: 3.

Preferably use of an agent or a WTX inhibitory agent in the methods of the invention results in increased expression or activity of a short isoform of a WTX polypeptide, or fragment or variants thereof, as compared to a full length, wild type WTX polypeptide isoform.

Preferably, selective increase in the transcription of one WTX mRNA as compared to a variant or alternative WTX mRNA transcribed from the same gene is achieved.

Preferably, selective increase in transcription of one WTX mRNA as compared to a variant or alternative WTX mRNA transcribed from the same gene is achieved through the use of a first agent and a second agent wherein the first agent inhibits one WTX mRNA transcribed from the gene and the second agent increases the expression of a variant or alternative WTX mRNA transcribed from the same gene.

More preferably, the first agent inhibits the expression or activity of a full length wild type WTX polypeptide and the second agent promotes expression or activity of a truncated WTX polypeptide of the invention. Most preferably, the first agent is a dsRNA, antisense compound, ribozyme or small molecule inhibitor that is a WTX inhibitory compound and the second agent is a recombinant vector that directs the overexpression of a truncated WTX polypeptide of the invention.

In one embodiment, the selective increase in the transcription of WTX one mRNA as compared to a variant or alternative WTX mRNA transcribed from the same gene is achieved in a cell, in vitro. Preferably the cell is a bone cell, more preferably an osteoprogenitor cell, a multipotent progenitor cell or an osteoblast cell. Preferably the selective increase in mRNA transcription as above is achieved in vivo.

In one embodiment, a first agent, useful in the inventive methods, that inhibits one variant or alternative mRNA transcribed from a single gene, is used in combination with a second agent, useful in the inventive methods, wherein the second agent increases the expression of a variant or alternative mRNA transcribed from the same gene. By way of non-limiting example, the first useful agent can be an agent that inhibits the expression or activity of a full length wild type WTX polypeptide and the second useful agent can be an agent that promotes expression or activity of a truncated WTX polypeptide of the invention. Preferably, the first useful agent is a dsRNA, antisense compound or small molecule inhibitor that is a WTX inhibitory compound and the second useful agent is a recombinant vector that allows for overexpression of a truncated WTX polypeptide of the invention.

### Antibodies

In one embodiment, an agent useful in the methods of the invention is an antibody capable of specifically binding to a WTX polypeptide and to fragments or variants of such antibodies. An antibody that binds to a WTX polypeptide, or a fragment or variant thereof, may be in any form, including all classes of polyclonal, monoclonal, single chain, human, humanized antibodies and chimeric antibodies produced by genetic recombination. Also included is antiserum obtained by immunizing an animal such as a mouse, rat or rabbit with a WTX polypeptide or a fragment or variant thereof.

A fragment of an antibody or a modified antibody may also be used herein so long as it binds a WTX polypeptide or a fragment or variant thereof. The antibody fragment may be Fab, F(ab′), F(ab′), and Fc or Fv fragment or single chain Fv (scFv), in which Fv fragments from H and L chains are ligated by an appropriate linker (Huston et al. Proc. Natl. Acad. Sci. USA 85:5879-83 (1988)). The “Fc” portion of an antibody refers to that portion of an immunoglobulin heavy chain that comprises one or more heavy chain constant region domains; CH1, CH2 and CH3, but does not include the heavy chain variable region.

Methods for preparing antibodies are well known in the art (see for example Harlow and Lane (1998)²⁸. Most commonly used antibodies are produced by immunizing a suitable host mammal. Fusion proteins comprising a WTX polypeptide may also be used as immunogens.

An antibody may be modified by conjugation with a variety of molecules, such as polyethylene glycol (PEG). The modified antibody can be obtained by chemically modifying an antibody. These modification methods are conventional in the field.

Alternatively, an antibody may be obtained as a chimeric antibody, between a variable region derived from nonhuman antibody and the constant region derived from human antibody, or as a humanized antibody, comprising the complementarity determining region (CDR) derived from nonhuman antibody, the frame work region (FR) derived from human antibody, and the constant region. Such antibodies can be prepared using known art methods.

In brief, methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a WTX polypeptide or a fragment or variant thereof or a fusion protein thereof. It may be useful to conjugate the immunizing agent to a protein known to be immunogenic in the mammal being immunized. Examples of such immunogenic proteins include but are not limited to keyhole limpet hemocyanin, bovine serum albumin, bovine thyroglobulin, and soybean trypsin inhibitor. Examples of adjuvants which may be employed include Freund's complete adjuvant and MPL TDM adjuvant (monophosphoryl Lipid A, synthetic trehalose dicorynomycolate). The immunization protocol may be selected by one skilled in the art without undue experimentation.

Monoclonal antibodies may be prepared using hybridoma methods well known in the art. See for example Kohler and Milstein, 1975²⁹ and U.S. Pat. No. 4,196,265. The hybridoma cells may be cultured in a suitable culture medium; alternatively, the hybridoma cells may be grown in vivo as ascites in a mammal. Preferred immortalized cell lines are murine myeloma lines, which can be obtained, for example, from the American Type Culture Collection, Virginia, USA. Immunoassays may be used to screen for immortalized cell lines which secrete the antibody of interest. Sequences of a WTX polypeptide or fragments or variants thereof may be used in screening.

Accordingly, also contemplated herein are hybridomas which are immortalized cell lines capable of secreting a WTX polypeptide specific monoclonal antibody.

Well known means for establishing binding specificity of monoclonal antibodies produced by the hybridoma cells include immunoprecipitation, radiolinked immunoassay (RIA), enzyme-linked immunoabsorbent assay (ELISA) and Western blot. (Lutz et al., Exp. Cell. Res. 175:109-124 (1988)). Samples from immunized animals may similarly be screened for the presence of polyclonal antibodies.

To facilitate detection, antibodies and fragments herein may be labeled with detectable markers such as fluorescent, bioluminescent, and chemiluminescent compounds, as well as radioisotopes, magnetic beads and affinity labels (e.g. biotin and avidin). Examples of labels which permit indirect measurement of binding include enzymes where the substrate may provide for a colored fluorescent product, suitable enzymes include horseradish peroxidase, alkaline phosphatase, malate dehydrogenase and the like. Fluorochromes (e.g. Texas Red, fluorescein, phycobiliproteins, and phycoerythrin) can be used with a fluorescence activated cell sorter. Labeling techniques are well known in the art.

The monoclonal antibodies secreted by the cells may be isolated or purified from the culture medium or ascites fluid by conventional immunoglobulin purification procedures such as, for example, protein A-Sepharose, hydroxyapatite chromatography, gel electrophoresis, dialysis, or affinity chromatography.

The monoclonal antibodies or fragments may also be produced by recombinant DNA means (see for example U.S. Pat. No. 4,816,567). DNA modifications such as substituting the coding sequence for human heavy and light chain constant domains in place of the homologous murine sequences (U.S. Pat. No. 4,816,567 above) are also possible. The antibodies may be monovalent antibodies. Methods for preparing monovalent antibodies are well known in the art. The production of chimeric, bivalent antibodies is also contemplated herein.

The antibodies useful according to the invention may further comprise humanized antibodies or human antibodies. Humanized antibodies include human immunoglobulins in which residues from a complementary determining region (CDR) of the recipient are replaced by residues from a CDR of a non-human species. The production of humanized antibodies from non-human sources such as rabbit, rat and mouse are well known.^{12,13,14}

Human antibodies can also be produced using various techniques known in the art, including phage display libraries¹⁵; and transgenic methods, see, for example Neuberger 1996³⁰; and Vaughan et al, 1998³¹.

Bispecific antibodies may also be useful. These antibodies are monoclonal, preferably human or humanized, antibodies that have binding specificities for at least two different antigens. For example a WTX polypeptide or a variant or fragment thereof, and another antigen that could be any antigen of a protein involved or associated with bone growth or formation. Antibodies with greater than two specificities for example trispecific antibodies are also contemplated herein.

Methods for making bispecific antibodies are known in the art. See for example Milstein and Cuello 1983³², Suresh et al., 1986³³ and Brennan et al., 1985³⁴.

A WTX polypeptide which is selectively bound by the antibody is a WTX polypeptide or an antigenic variant or fragment thereof as discussed above.

Preferably, the antibody binds a full length wild type WTX polypeptide (including fragments or variants thereof) or a truncated WTX polypeptide of the invention, (including fragments or variants thereof) and inhibits WTX polypeptide activity.

Antibody binding of a WTX polypeptide can be detected by any means known in the art including specific (antibody based) and non specific (such as HPLC solid phase). Most commonly, antibodies herein are detected using an assay such as ELISA or RIA as noted above. Competitive binding assays, sandwich assays, non-competitive assays, fluoroimmunoassay, immunofluorometric assay, or immunoradiometric assays, luminescence assays, chemiluminescence assays and mass spectrometry analysis such a surface-enhanced laser desorption and ionization (SELDI) electrospray ionization (ESI), matrix assisted laser-desorption ionization (MALDI), Fourier transform Ion cyclotron resonance mass spectroscopy (FTICR) alone or in combination with non-specific binding agents such as chromatography formats are also feasible.

Conveniently, an antibody can be fixed to a solid substrate to facilitate washing and isolation of a WTX polypeptide/antibody complex. Binding of antibodies to a solid support can be achieved using known art techniques. See for example Handbook of Experimental Immunology, 4th edition, Blackwell Scientific Publications, Oxford (1986). Useful solid substrates for antibodies include glass, nylon, paper and plastics. Similarly, a WTX polypeptide can be adsorbed onto a solid substrate such as adsorbent silica, or resin particles, or silicon chips optionally coated or derivatized with ion exchange, reverse phase (e.g. C18 coating) or other materials. The substrate may be in the form of beads, plates, tubes, sticks or biochips. Biochips or plates with addressable locations and discreet microtitre plates are particularly useful. Also preferred for use are multiple systems where beads containing antibodies directed to multiple analytes are used to measure levels of the analytes in a single sample. Analytes to be measured may include other proteins related to bone disease or conditions as well as a WTX polypeptide or variants or fragments thereof. One example of a suitable multiplex bead system for use herein is the Luminex Fluorokine Multianalyte Profiling system.

Antibody assay methods are well known in the art see for example U.S. Pat. No. 5,221,685, U.S. Pat. No. 5,310,687, U.S. Pat. No. 5,480,792, U.S. Pat. No. 5,525,524, U.S. Pat. No. 5,679,526, U.S. Pat. No. 5,824,799, U.S. Pat. No. 5,851,776, U.S. Pat. No. 5,885,527, U.S. Pat. No. 5,922,615, U.S. Pat. No. 5,939,272, U.S. Pat. No. 5,647,124, U.S. Pat. No. 5,985,579, U.S. Pat. No. 6,019,944, U.S. Pat. No. 6,113,855, U.S. Pat. No. 6,143,576 and for unlabelled assays U.S. Pat. No. 5,955,377, and U.S. Pat. No. 5,631,171 see also Zola, Monoclonal Antibodies: A Manual of Techniques pp 147-158 (CRC Press, Inc 1987), Harlow and Lane (1998) Antibodies, A Laboratory Manual, Cold Spring Harbour Publications, New York, and US 2005/0064511 for a description of assay formats and conditions all of the above references are incorporated herein by reference in their entirety.

Immunoassay analyzers are also well known and include Beckman Access, Abbott AxSym, Roche ElecSys and Dade Behring Status systems amongst others which are well described.

Binding of a WTX polypeptide and an antibody to form a complex can be detected directly or indirectly. Direct detection is carried out using labels such as fluorescence, luminescence, radionuclides, metals, dyes and the like. Indirect detection includes binding detectable labels such as digoxygenin or enzymes such as horseradish peroxidase and alkaline phosphatase to form a labeled WTX polypeptide antibody followed by a step of detecting the label by addition of detection reagents.

Horseradish peroxidase for example can be incubated with substrates such as o-Phenylenediamine Dihyhydrochloride (OPD) and peroxide to generate a colored product whose absorbance can be measured, or with luminol and peroxide to give chemiluminescent light which can be measured in a luminometer as is known in the art. Biotin or digoxygenin can be reacted with binding agents that bind strongly to them. For example, the proteins avidin and streptavidin will bind strongly to biotin. A further measurable label is then covalently bound or linked thereto either by direct reaction with the protein, or through the use of commonly available crosslinking agents such as MCS and carbodimide, or by addition of chelating agents.

Generally, the complex is separated from the uncomplexed reagents for example by centrifugation. If the antibody is labeled, the amount of complex will be reflected by the amount of label detected. Alternatively, a WTX polypeptide may be labeled by binding to an antibody and detected in a competitive assay by measuring a reduction in bound labeled WTX polypeptide when the antibody-labeled-WTX polypeptide is incubated with a biological sample containing unlabelled WTX polypeptide. Other immunoassays may be used for example a sandwich assay.

In one example, following contact with the antibody, usually overnight for 18 to 25 hours at 4° C., or for 1 to 2 to 4 hours at 25° C. to 40° C., the labeled WTX polypeptide bound to the binding agent (antibody) is separated from the unbound labeled WTX polypeptide. In solution phase assays, the separation may be accomplished by addition of an anti gamma globulin antibody (second-antibody) coupled to solid phase particles such as cellulose, or magnetic material. The second-antibody is raised in a different species to that used for the primary antibody and binds the primary antibody. All primary antibodies are therefore bound to the solid phase via the second antibody. This complex is removed from solution by centrifugation or magnetic attraction and the bound labeled peptide measured using the label bound to it. Other options for separating bound from free label include formation of immune complexes, which precipitate from solution, precipitation of the antibodies by polyethylene glycol or binding free labeled peptide to charcoal and removal from solution by centrifugation of filtration. The label in the separated bound or free phase is measured by an appropriate method such as those presented above.

Competitive binding assays can also be configured as solid phase assays that are easier to perform and are therefore preferable to those above. This type of assay uses plates with wells (commonly known as ELISA or immunoassay plates), solid beads or the surfaces of tubes. The primary antibody is either adsorbed or covalently bound to the surface of the plate, bead or tube, or is bound indirectly through a second anti gamma globulin or anti Fc region antibody adsorbed or covalently bound to the plate. Sample and labeled peptide (as above) are added to the plate either together or sequentially and incubated under conditions allowing competition for antibody binding between a WTX polypeptide in the sample and the labeled peptide. Unbound labeled peptide can subsequently be aspirated off and the plate rinsed leaving the antibody bound labeled peptide attached to the plate. The labeled peptide can then be measured using techniques described above.

Sandwich type assays are more preferred for reasons of specificity, speed and greater measuring range. In this type of assay an excess of the primary antibody to a WTX polypeptide is attached to the well of an ELISA plate, bead or tube via adsorption, covalent coupling, or an anti Fc or gamma globulin antibody, as described above for solid phase competition binding assays. Sample fluid or extract is contacted with the antibody attached to the solid phase. Because the antibody is in excess this binding reaction is usually rapid. A second antibody to a WTX polypeptide is also incubated with the sample either simultaneously or sequentially with the primary antibody. This second antibody is chosen to bind to a site on a WTX polypeptide that is different from the binding site of the primary antibody. These two antibody reactions result in a sandwich with a WTX polypeptide from the sample sandwiched between the two antibodies. The second antibody is usually labeled with a readily measurable compound as detailed above for competitive binding assays. Alternatively a labeled third antibody which binds specifically to the second antibody may be contacted with the sample. After washing away the unbound material the bound labeled antibody can be measured and quantified by methods outlined for competitive binding assays.

A dipstick type assay may also be used. These assays are well known in the art. They may for example, employ small particles such as gold or colored latex particles with specific antibodies attached. The liquid sample to be measured may be added to one end of a membrane or paper strip preloaded with the particles and allowed to migrate along the strip. Binding of the antigen in the sample to the particles modifies the ability of the particles to bind to trapping sites, which contain binding agents for the particles such as antigens or antibodies, further along the strip. The accumulation of the colored particles at these sites results in color development and is dependent on the concentration of competing antigen in the sample. Other dipstick methods may employ antibodies covalently bound to paper or membrane strips to trap antigen in the sample. Subsequent reactions employing second antibodies coupled to enzymes such as horse radish peroxidase and incubation with substrates to produce color, fluorescent or chemiluminescent light output will enable quantification of antigen in the sample.

As discussed in the following examples, radioimmunoassay (RIA) is a currently preferred laboratory technique. In one RIA a radiolabeled antigen and unlabelled antigen are employed in competitive binding with an antibody. Common radiolabels include ¹²⁵I, ¹³¹I, ³H and ¹⁴C.

Radioimmunoassays involving precipitation of a WTX polypeptide with a specific antibody and radiolabeled antibody binding protein can measure the amount of labeled antibody in the precipitate as proportional to the amount of a WTX polypeptide in the sample. Alternatively, a labeled a WTX polypeptide is produced and an unlabelled antibody binding protein is used. A biological sample to be tested is then added. The decrease in counts from the labeled a WTX polypeptide is proportional to the amount of a WTX polypeptide in the sample.

In RIA it is also feasible to separate bound WTX polypeptide from free WTX polypeptide. This may involve precipitating a WTX polypeptide/antibody complex with a second antibody. For example, if a WTX polypeptide antibody complex contains rabbit antibody then donkey anti-rabbit antibody can be used to precipitate the complex and the amount of label counted. For example in an LKB, Gammamaster counter. See Hunt et al³⁵.

### Antisense Compounds

In the following discussion, where reference is made an antisense oligonucleotide or to antisense oligonucleotides, it is to be understood by the reader that the applicable discussion and disclosure applies equally to all antisense compounds useful in the methods of the present invention, either as known in the art or as described herein.

In another embodiment, an agent useful in the methods of the invention is an antisense compound. An antisense compound can be any an antisense molecule that is complementary to a polynucleotide sequence encoding a polypeptide of the invention wherein said antisense molecule inhibits the expression of the polypeptide of the invention.

An antisense compound is said to be “antisense” when it is complementary to and hybridizes with a target nucleic acid or nucleotide sequence. The targeting of an antisense compounds involves the identification of a nucleic acid sequence whose function is to be modulated and determination of a suitable target site or sites within the nucleic acid sequence. Subsequent hybridization of the antisense compound is carried out and results in modulation of gene expression. As known in the art, an antisense compound may specifically hybridize to a target nucleic acid sequence in the absence of 100% complementarity, as discussed above with reference to nucleic acid hybridization. The process of targeting of antisense compounds to particular nucleic acid sequences is well known and understood in the art as disclosed, for example, in U.S. Pat. No. 5,686,242, U.S. Pat. No. 5,801,154, U.S. Pat. No. 6,159,614, U.S. Pat. No. 6,248,586, Agarwal, TIBECH, 14:376-387 (1996) and Mahato et al., Expert Opinion on Drug Delivery, 2(1):3-28 (2005), all of which are herein incorporated by reference.

Specific hybridization occurs where binding of the antisense compound to the target interferes with the normal function of the target, for example, translation of an mRNA, but not limited thereto. Other functions interfered with can include translocation of the RNA within a cell, RNA splicing, RNA catalytic activity and interference with RNA specific binding proteins. Specific hybridization avoids non-specific binding of the oligonucleotide to non-target sequences under the desired conditions. Desired conditions may include in vitro or in vivo physiological conditions, such as in the case of assays or therapeutic treatment.

In targeting a particular mRNA, the skilled person understands that a typical mRNA includes a protein coding region, a 5′-untranslated region, a 3′-untranslated region, a 5′ cap region and may include intron/exon junctions. An antisense compound may be designed to specifically hybridize to any suitable nucleic acid sequence within any of these regions. An antisense compound useful in the methods of the present invention will specifically hybridize to a WTX polynucleotide sequence comprised in SEQ ID NO: 1. In one embodiment, the antisense compound is an antisense oligonucleotide. Preferably the antisense oligonucleotide will specifically hybridize to a target sequence comprised in SEQ ID NO: 1. In a preferred embodiment, the antisense oligonucleotide will specifically hybridize to a target sequence comprised between nucleotide positions 151 and 980 of SEQ ID NO: 1.

Antisense compounds can be targeted effectively, as known in the art, to particular preferred regions of a nucleic acid sequence. Such regions include, for example, regions of 25 to 50 contiguous nucleotides in an mRNA in either direction (5′ or 3′) surrounding a translation initiation codon, a translation termination codon, the open reading frame (ORF) or “coding region,” the 5′ untranslated region (5′UTR), the 3′ untranslated region (3′UTR) and the 5′ cap region. A splice site in an mRNA, such as an exon-exon or intron-exon junctions may also be targeted by an antisense compound. Targeting of a splice side may be particularly useful where aberrant splicing of an mRNA is related to disease, including where the overproduction of a specific mRNA splice variant is related to a disease. Likewise, antisense compounds may be targeted to particular exons in an mRNA splice variant.

An antisense oligonucleotide can also be used in diagnostics, prophylaxis, as a research reagent or in a kit. Antisense oligonucleotides, for example, that hybridize to nucleic acids encoding WTX can be designed and used in, sandwich, colorimetric and other assays as described herein. When used for research purposes the specific hybridization of a WTX targeted antisense oligonucleotide may be useful in for assays, purifications, cellular product preparations and in other methodologies which may be appreciated by persons of ordinary skill in the art.

In the context of this invention, the term “oligonucleotide” is used as known and described in the art (including as disclosed and previously incorporated by reference herein) and refers to an oligomer or polymer of ribonucleic acid or deoxyribonucleic acid. Briefly, the term includes oligonucleotides constructed with both naturally-occurring and non-naturally occurring nucleobases, sugars and covalent internucleoside linkages. Desirable properties of modified or substituted oligonucleotides include, for example, enhanced cellular uptake, enhanced binding to target and increased stability in the presence of nucleases.

Antisense compounds useful in the methods of this invention may comprise from about 8 to about 80 nucleobases. In one embodiment the antisense compound is an antisense oligonucleotide comprising from about 8, 9, 10, 11, 12, 13, 14, 15, 16, 17,18, 19, 20, 21, 22, 23, 24, 25, 26, 27, 28, 29 or 30 nucleobases (i.e. comprising from about 8, 9, 10, 11, 12, 13, 14, 15, 16, 17, 18, 19, 20, 21, 22, 23, 24, 25, 26, 27, 28, 29 or 30 linked nucleosides).

Nucleoside means a base-sugar combination. The base portion of the nucleoside may be a heterocyclic base such as a purine or a pyrimidine. Nucleotide means a nucleoside that includes a phosphate group covalently linked to the nucleoside sugar moiety. The normal linkage or backbone of an RNA or DNA molecule is a 3= to 5=phosphodiester linkage.

As is well known in the art, modified oligonucleotide backbones containing a phosphorus atom therein include, for example, phosphorothioates, chiral phosphorothioates, phosphoro-dithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

An antisense compound can be modified to include at least one phosphorothioate bond. Inclusion of a phosphorothioate bond is may be useful to induce stimulation of a local immune response. This is known in the art and described in U.S. Pat. No. 5,663,153, which is herein incorporated by reference. The immunostimulatory effect of a modified antisense oligonucleotide may be useful either alone or in combination with an additional therapeutic agent, for example another drug. Immunostimulation may also be useful in combination with other therapies, such as surgical procedures.

Additional modified antisense oligonucleotides are known in the art as antisense oligonucleosides. Such oligonucleosides comprise internucleoside backbones that lack a phosphorus atom therein where the internucleoside backbone is formed by short chain alkyl or cycloalkyl internucleoside linkages, mixed heteroatom and alkyl or cycloalkyl internucleoside linkages, or one or more short chain heteroatomic or heterocyclic internucleoside linkages. Included in this group are antisense oligonucleotides that have morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts. Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference. Additional modified antisense compounds include morpholino nucleic acids (heterocyclic bases attached to the morpholino ring), Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety and analogs thereof, tricyclic nucleoside analogs and phosphonomonoester nucleic acids and peptide nucleic acids (PNA) (Singh et al., Chem. Commun., 1998, 4, 455-456; Wahlestedt et al., Proc. Natl. Acad. Sci. U.S.A., 2000, 97, 5633-5638.)

Antisense compounds useful in the methods of the present invention can also contain one or more substituted sugar moieties as known and described in the art. The skilled worker can select from amongst many know sugar substituent groups commonly used in the art of antisense compound modification including various chemical moieties and functional groups such as OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are O[(CH2)nO]mCH3, O(CH2)nOCH3, O(CH2)nNH2, O(CH2)nCH3, O(CH2)nONH2, and O(CH2)nON[(CH2)nCH3]2, where n and m are from 1 to about 10, C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator and groups that improve pharmacokinetic or pharmacodynamic properties of an oligonucleotide. Well known and used modifications include 2′-O-(2-methoxyethyl) or 2′-MOE), 2′-dimethylaminooxyethoxy or 2′-DMAOE, 2′-O-dimethyl-amino-ethoxy-ethyl or 2′-DMAEOE, or a 2′-arabino modification such as 2′-F.

2-methoxyethoxy (2′-MOE, 2′-OCH2CH2OCH3) side chain modification of an antisense compound can increase nuclease resistance and binding affinity (Baker et al., J. Biol. Chem., 1997, 272, 11944-12000). The binding affinity of antisense compounds having a 2′-MOE substitution is greater than such compounds having many similar 2′ modifications such as O-methyl, O-propyl, and O-aminopropyl. Antisense oligonucleotides having the 2′-O-methoxyethyl substituent have been shown potent inhibitors of gene expression and show promise for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926). Relative to DNA, oligonucleotides having the 2′-MOE modification display improved RNA affinity and higher nuclease resistance.

Similar modifications may be made at other positions on the antisense compound including the sugar 3′ position on a 3′ terminal nucleoside or in a 2′-5′ linked oligonucleotides and the 5′ position of 5′ terminal nucleotide. Oligomeric compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

Antisense compounds can include nucleobase modifications or substitutions as well known and used in the art. Nucleobase modifications as used herein refer to modifications of “unmodified” or “natural” nucleobases where the “unmodified” or “natural” nucleobases the purine bases adenine (A) and guanine (G), and the pyrimidine bases thymine (T), cytosine (C) and uracil (U). Modified nucleobases may include any kind of synthetic and natural nucleobases as known and used in the art such as such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Modified nucleobases can also refer to nucleobases where “unmodified”: or “natural” base is replaced, such as in 7-deaza-adenine, 7-deazaguanosine, 2-aminopyridine and 2-pyridone. Numerous different nucleobases are disclosed in U.S. Pat. No. 3,687,808; The Concise Encyclopedia Of Polymer Science And Engineering, pages 858-859, Kroschwitz, J. I., ed. John Wiley & Sons, 1990; Englisch et al., Angewandte Chemie, International Edition, 1991, 30, 613 and Sanghvi, Y. S., Chapter 15, Antisense Research and Applications, pages 289-302, Crooke, S. T. and Lebleu, B., ed., CRC Press, 1993 each of which is incorporated herein by reference).

One particularly useful nucleobase modification, is a 5-methylcytosine substitution to increase nucleic acid duplex stability. Preferably a 5-methylcytosine substitution can be combined with 2′-O-methoxyethyl sugar modifications in a useful antisense oligonucleotide.

Antisense compounds can be modified by the linkage of one or more moieties or conjugates which enhance the properties of activity, cellular distribution or cellular uptake. Useful antisense compounds can be prepared by covalent attachment of functional groups as known and used in the art. Examples of such groups include hydroxyl or amino groups, intercalators, reporter molecules, polyamines, polyamides, polyethylene glycols, polyethers, and groups that enhance pharmacokinetic or pharmacodynamic properties. Some examples of particular conjugate groups are, but are not limited to, cholesterols, lipids, phospholipids, biotin, phenazine, folate, phenanthridine, anthraquinone, acridine, fluoresceins, rhodamines, coumarins, and other dyes.

Groups that enhance the pharmacokinetic or pharmacodynamic properties, in the context of this invention, include groups that may improve or enhance one or all of cellular uptake, resistance to degradation, sequence-specific hybridization with RNA, distribution, metabolism or excretion of an antisense compound. Particular conjugate moieties may be, but are not limited to, cholesterol (Letsinger et al., Proc. Natl. Acad. Sci. USA, 1989, 86, 6553-6556), a phospholipid, a polyamine or a polyethylene glycol chain (Manoharan et al., Nucleosides & Nucleotides, 1995, 14, 969-973).

Additional modifications which may be made to the antisense compounds of the invention, particularly in the context of therapeutic applications, include but are not limited to antisense oligonucleotides modified to have enhanced membrane permeability and stability. Such modifications may be made through conjugating, for example, oligoribonucleotides with a carrier agent including 2,4-dinitrophenyl-(DNP) or 3-fluoro-4,6-dinitrophenyl-(FDNP) groups coupled at the 2′-O position of the oligoribonucleotide as disclosed in U.S. Pat. No. 5,496,546, U.S. Pat. No. 5,858,988 and U.S. Pat. No. 6,291,438, each of which is hereby incorporated by reference in its entirety.

Other conjugates moieties can include drugs such as, aspirin, ibuprofen, a diazepine, a barbiturate, a cephalosporin, a sulfa drug, an antidiabetic, an antibacterial or an antibiotic, but are not limited thereto. Representative United States patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

Various modifications of an antisense oligonucleotide may be made, and as known in the art, not all modifications are required to be the same. Various combinations of modifications may be made to the nucleobases, sugar and internucleoside portion of the antisense compound. Where an antisense oligonucleotide contains two or more chemically distinct regions, each made up of at least one monomer unit, i.e., a nucleotide, that antisense compound is referred to as a chimeric antisense oligonucleotide. Such antisense oligonucleotides typically contain at least one region modified to confer increased resistance to nuclease degradation, increased cellular uptake, and/or increased binding affinity for the target nucleic acid. An additional region may be modified, for example, to present a substrate for enzymes capable of cleaving RNA:DNA or RNA:RNA hybrids. A chimeric antisense compound can be a composite of two or more oligonucleotides, analogs and/or mimetics as described previously. Such chimeric antisense compound are known and used in the art of antisense inhibition of gene expression and are referred to as hemimers, gapmers or inverted gapmers. An example of a gapmer is a chimeric oligonucleotide having 2′-MOE substituents in the wing nucleosides and an internal region of deoxy-phosphorothioate nucleotides. Antisense oligonucleotides of this configuration are known to be effective at reducing the growth of tumors in animal models at low doses. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

Antisense compounds can include synthetically modification of nucleoside bases, sugars or both to produce nucleosides that induce a 3′-endo sugar conformation. Such nucleosides may mimic RNA like nucleosides allowing some properties, e.g., pharmacodynamic or pharmacokinetic properties, to be enhanced while maintaining the desirable 3′-endo conformational geometry. An RNA type duplex (A form helix, predominantly 3′-endo) may be a requirement (e.g. trigger) of RNA interference.

Enhanced pharmacodynamic and pharmacokinetic properties can, but are not limited to protein binding, protein off-rate, absorption and clearance; modulation of nuclease stability and chemical stability; modulation of binding affinity and specificity and efficacy of RNA cleavage.

Antisense compounds can act as triggers of RNAi where they comprise one or more nucleosides modified in such a way as to favor a C3′-endo type conformation. Examples of such triggers include, but are not limited to, Locked Nucleic Acid (LNA, Singh et al, Chem. Commun. (1998), 4, 455-456), and ethylene bridged Nucleic Acids (ENA, Morita et al, Bioorganic & Medicinal Chemistry Letters (2002), 12, 73-76.)

The preferred conformation of antisense compounds comprising modified nucleosides can be estimated by various methods as known in the art such as molecular dynamics calculations, nuclear magnetic resonance spectroscopy and CD measurements. These methods allow selection of particular modifications predicted to induce A-form duplex geometry (RNA like conformations).

Antisense compounds having enhanced properties compared to native RNA against nucleic acid targets are prepared as known in the art. Briefly, a target is identified a complementary antisense oligonucleotide is selected and each nucleoside of the oligonucleotide considered for possible modifications. Preferred modifications are those that replace one or more RNA nucleosides with nucleosides having the same 3′-endo conformational geometry. The antisense oligonucleotide can also be evaluated for modifications that result in a chimeric configuration. Advantageous modifications are often made to one or more of the nucleoside residues of the 5′ and 3′-termini. Further modifications of internucleoside linkages, conjugate groups, substitute sugars or bases, substitution of one or more nucleosides with nucleoside mimetics and any other property enhancing modification are also considered.

In general, any modification of a nucleobases, sugar moiety or internucleoside linkage of a antisense compound that increases nucleic acid duplex stability, enhances the binding affinity of the compound to a target sequence, increases the capability of the compound activate RNaseH, enhances cellular uptake of the compound, increase resistance of the compound to degradation or otherwise promotes an increased antisense activity of the compound, particularly in vivo, is contemplated for the antisense compounds useful in the methods of the present invention.

### RNA Interference

In addition to single-stranded antisense compounds, i.e., antisense oligonucleotides, agents useful in the methods of the invention include double-stranded structures, such as double-stranded RNA (dsRNA) molecules. dsRNA is known to induce potent and specific gene silencing in both plants and animals. Such gene silencing is known in the art as post-transcriptional gene silencing and is thought to be an evolutionarily-conserved cellular defense mechanism used to prevent the expression of foreign genes and is commonly shared by diverse flora and phyla (Fire et al, 1999, Trends Genet., 15, 358). Gene silencing due to dsRNA is well known in the art and is described, for example, in the following references: Guo and Kempheus, Cell, 1995, 81, 611-620, Montgomery et al., Proc. Natl. Acad. Sci. USA, 1998, 95, 15502-15507; Fire et al., Nature, 1998, 391, 806-811 and Tijsterman et al., Science, 2002, 295, 694-697).

dsRNA molecules including, siRNA and shRNA molecules that modulate the expression or activity of a WTX polynucleotide or polypeptide are useful in the methods of the invention. Such dsRNA molecules can be designed to target similar regions as those targeted by antisense compounds and will have similar effects. dsRNA molecules are generally 18 to 25 base pairs in length, but may range between 8 and 50 nucleobase pairs, preferably 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleobase pairs in length. Most preferably a dsRNA molecule is 19-21 base pairs in length. By way of example, each strand of an siRNA that is 21 base pairs in length will consist of 23 nucleotide residues with 21 complementary residues and a 2 nucleotide residue overhang. A designed dsRNA molecule, for example an siRNA targeted to a polynucleotide that encodes a WTX polypeptide, may be synthesized by conventional oligonucleotide synthesis techniques. Where the dsRNA molecule that targets a polynucleotide encoding a WTX polypeptide is an shRNA then the shRNA may be expressed from one or more vectors as described herein and as known in the art.

Each individual strand of a dsRNA may be transcribed from an expression cassette comprising the same or different promoters, wherein each expression cassette may be comprised in a separate recombinant vector (which can be the same or different recombinant vectors) and wherein the two individual strands are then co-transfected into a target cell. Each individual strand of a dsRNA may also be operably linked to a promoter, which can be the same or a different promoter, where transcription of each individual RNA strand is from the same expression cassette. A dsRNA is preferably expressed as inverted repeats joined by a linker polynucleotide sequence such that the dsRNA has a stem and loop structure.

A dsRNA may also comprise a non-nucleic acid “linker” which covalently joins the 3″-end of one strand and the 5″end of the respective other strand. The two strands of a dsRNA can have the same or a different number of nucleotides. The maximum number of base pairs in the dsRNA equals the nucleotide number of the shortest strand not including overhangs.

A certain number of mismatches may be tolerated where an antisense strand is not fully complementary to a given target sequence. Mismatches most tolerated are in the terminal regions of a dsRNA duplex formed between an antisense strand and a target sequence and, if present, are generally within 6, 5, 4, 3, or 2 nucleotides of the 5′ and/or 3′ terminus of duplex, not including nucleotide overhangs.

As used herein, a dsRNA may comprise one or more nucleotide overhangs. A dsRNA can be designed so that at least one end of the dsRNA has a single-stranded nucleotide overhang of 1 to 4, preferably 1 or 2 nucleotides. dsRNAs having at least one nucleotide overhang have superior inhibitory properties as compared to their blunt-ended equivalents. Generally, the single-stranded overhang is located at the 3′-terminal end of the antisense strand or, alternatively, at the 3′-terminal end of the sense strand. The dsRNA may also have a blunt end, generally located at the 5′-end of the antisense strand.

Such dsRNAs have improved stability and inhibitory activity, thus allowing administration at low dosages, i.e., less than 5 mg/kg body weight of the recipient per day. In some instances, the presence of a single nucleotide overhang strengthens the interference activity of the dsRNA, without affecting its overall stability and may provide a dsRNA that is particularly stable and effective in vivo. (WO08036933). Generally, the antisense strand of the dsRNA has a nucleotide overhang at the 3′-end, and the 5′-end is blunt. In another embodiment, one or more of the nucleotides in the overhang is replaced with a nucleoside thiophosphate.

Single stranded overhangs of a dsRNA that are designed to form upon hybridization of two RNA strands are not “mismatches” when determining complementarity. By way of non-limiting example, a dsRNA comprising a first oligonucleotide of 18 to 25 nucleotides in length and a second oligonucleotide of 18 to 25 nucleotides in length where the second oligonucleotide is 2 nucleotides shorter and hybridizes along the entire length of the first oligonucleotide, can be said, for the purposes of the invention, to be fully complementary.

In one embodiment, a dsRNA useful in the methods of the invention is a dsRNA that inhibits the expression of said WTX polypeptide. The dsRNA may be delivered directly to a particular cell or tissue or it may be expressed in a particular cell or tissue from one or more expression vectors encoding said dsRNA molecule.

Vector encoded dsRNA agents useful in the methods of the invention may be expressed from recombinant vectors, either in vitro, for subsequent delivery as known in the art and described herein, or, intracellularly, in vivo. Preferably the recombinant vector is a recombinant plasmid or recombinant viral vector. More preferably the recombinant vector is a recombinant viral vector as known and used in the art.

Any suitable promoter may be operably linked to a polynucleotide to be expressed and used in accordance with the methods of the invention, to direct expression of a desired dsRNA from either a recombinant DNA plasmid or recombinant viral vector. Non-limiting examples of suitable promoters include, a eukaryotic RNA polymerase I (e.g. ribosomal RNA promoter), RNA polymerase II (e.g. CMV early promoter or actin promoter or Ul snRNA promoter), RNA polymerase III promoter (e.g. U6 snRNA, H1 RNA or 7SK RNA promoter) or a prokaryotic promoter, for example the T7 promoter. If a T7 promoter is chosen then the vector must also encode the T7 RNA polymerase required for transcription from a T7 promoter.

The selection of additional suitable promoters is within the skill in the art. Suitable recombinant viral vectors may comprise an inducible or regulatable promoter that allows for controlled expression of the dsRNA in a particular cell, tissue or specified intracellular environment. Inducible regulatory sequences and expression systems that are sensitive to certain physiological regulators, e.g., circulating glucose levels, or hormones, are known in the art (Docherty et al., 1994, FASEB J. 8:20-24).

The skilled person may choose the appropriate regulatory/promoter sequence based on the intended use of the dsRNA to be expressed. The use of a particular inducible or regulatable promoter will depend on the particular cell, cell type, tissue or organism in which transcription is to be controlled. Non-limiting examples of expression systems comprising suitable promoters include expression systems regulated by ecdysone, estrogen, progesterone, tetracycline, chemical inducers of dimerization, and isopropyl-beta-D1-thiogalactopyranoside (EPTG) and the like, as known in the art.

A useful dsRNA agent may be expressed from a recombinant viral vector may be as two separate, complementary RNA strands, or as a single RNA strand comprising an inverted repeat joined by a linker polynucleotide sequence such that the dsRNA has a stem and loop structure, e.g., a hairpin loop.

Any suitable viral vector capable of accepting the coding sequences for the dsRNA molecule(s) to be expressed can be used and will be, upon accepting said coding sequences, considered herein, a recombinant viral vector. Exemplary viral vectors that are considered suitable for use in the inventive methods are, but are not limited to, vectors derived from: adenovirus (AV); adeno-associated virus (AAV); retroviruses (e.g., lentiviruses (LV), Rhabdoviruses, murine leukemia virus) and herpes virus.

Such viral vectors can be pseudotyped as known and used in the art using modified envelope proteins or other surface antigens from other viruses, or by substituting different viral capsid proteins, as required.

In one example, construction of a suitable AAV vector which expresses different capsid protein serotypes falls within the skill in the art; see, e.g., Rabinowitz J E et al. (2002), J Virol 76:791-801.

The selection of an appropriate recombinant viral vector, methods for inserting polynucleotide sequences into the vector, and methods of viral vector delivery are considered to be within the skill in the art. (Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference).

In one embodiment, a suitable viral vector useful to deliver a dsRNA according to the inventive methods of the invention is an AAV derived recombinant viral vector comprising a polynucleotide encoding a useful dsRNA agent that is expressed as two separate, complementary RNA strands wherein the polynucleotide to be expressed is operably linked to a U6 or H1 RNA, or CMV promoter.

The construction and delivery of such an AAV vector into target cells is also considered to be within the skill of those in the art (Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; entire disclosures of which are herein incorporated by reference). Recombinant adenoviral vectors can be used to infect a wide variety of cells and tissues in susceptible hosts (e.g., rat, hamster, dog, and chimpanzee) (Hsu et al., 992, J. Infectious Disease, 166:769). Such AAV vectors can be used advantageously in the methods of the invention as they do not require actively mitotic cells for effective delivery via infection/transduction.

Recombinant retroviral vectors capable of transducing and expressing genes inserted into the genome of a cell are also known and used in the art and can be produced by transfecting the recombinant retroviral genome into suitable packaging cell lines such as PA317 and Psi-CRIP (Comette et al., 1991, Human Gene Therapy 2:5 10; Cone et al., 1984, Pioc. Natl Acad. Sci. USA 8 1:6349).

Generally, recombinant vectors capable of expressing dsRNA molecules are delivered as herein and may persist in target cells. Alternatively, recombinant viral vectors may be used to provide for transient expression of dsRNA molecules. Such vectors can be administered repeatedly as necessary.

A dsRNA may also be delivered to a target region or tissue of a subject wherein the dsRNA is comprised in a cell that has been ex-planted from a subject and transformed or transduced with a gene therapy vector as previously described herein. The ex-planted and transformed or transduced cell is subsequently reintroduced into the subject, thereby delivering the dsRNA to the target tissue or region of the subject, as known in the art and as described herein.

In one embodiment, the dsRNA is an isolated siRNA or shRNA comprising a sense RNA strand and an antisense RNA strand, wherein the sense and the antisense RNA strands form an RNA duplex, and wherein the sense RNA strand comprises a nucleotide sequence that is substantially identical to a target sequence of about 18 to about 25 contiguous nucleotides in a WTX mRNA. Preferably the sense RNA strand comprises a nucleotide sequence that is identical to the target sequence.

In one embodiment, the dsRNA is an siRNA or shRNA that targets and specifically hybridizes to a human WTX mRNA comprising SEQ ID NO: 1. More preferably, the siRNA or shRNA targets and specifically hybridizes to SEQ ID NO: 1 between nucleotide residues at positions 150 and 981.

In another embodiment, the dsRNA is an siRNA or shRNA that targets and specifically hybridizes to a mouse WTX mRNA comprising SEQ ID NO: 25. In a preferred embodiment, the siRNA or shRNA targets and specifically hybridizes to SEQ ID NO: 25 between nucleotide at positions 150 to 981.

In one embodiment, the siRNA or shRNA is selected from the group consisting of an siRNA or shRNA having a sense strand that comprises SEQ ID NO: 29 and an antisense strand that comprises SEQ ID NO: 30 and an siRNA or shRNA having a sense strand that comprises SEQ ID NO: 31 and an antisense strand that comprises SEQ ID NO: 32

In a preferred embodiment the siRNA comprises a sense strand comprising SEQ ID NO: 45 and an antisense strand comprising SEQ ID NO: 46 or a sense strand comprising SEQ ID NO: 47 and an antisense strand comprising SEQ ID NO: 48. Most preferably the siRNA comprises a sense strand consisting of SEQ ID NO: 45 and an antisense strand consisting of SEQ ID NO: 46 or a sense strand consisting of SEQ ID NO: 47 and an antisense strand consisting of SEQ ID NO: 48,

A dsRNA agent useful in the methods of the can be chemically synthesized, expressed from a vector or synthesized enzymatically. A dsRNA agent may include chemical modifications to ribonucleotides, including modifications to single or multiple nucleotides. Modifications to dsRNA, as used herein, encompass all types of modifications as disclosed herein as well as all modifications known and used in the art. Any such modifications may also be made to an siRNA type molecule used in accordance with the invention.

The use of a chemically-modified dsRNA agent is known in the art to improve various properties of native dsRNA molecules through increased resistance to nuclease degradation in vivo and/or through improved cellular uptake (US2007/0185049). Chemically modified dsRNA having a longer half-life in serum or in cells or tissues can provide greater in vivo stability and bioavailability as compared to unmodified RNA molecules that are delivered exogenously, enabling delivery of a lower dose of a particular dsRNA to provide a desired therapy (US2007/0185049). In addition, certain chemical modifications as known in the art can improve the bioavailability and/or potency of dsRNAs by facilitating the targeting of nucleic acid molecules to particular organs, cells or tissues and/or improving cellular uptake of the nucleic acid molecules. Therefore, the overall activity of a chemically modified dsRNA can be greater in vivo than that observed for an unmodified dsRNA, even if the activity of the chemically modified dsRNA is reduced in vitro as compared to an equivalent, unmodified dsRNA (US2007/0185049).

In general, any modification of a nucleobases, sugar moiety or internucleoside linkage as disclosed herein with reference to an antisense compound can be made to a dsRNA as long as that modification enhances or improves desirable pharmacodynamic or pharmacokinetic properties of the dsRNA, including but not limited to, increased stability, enhanced binding affinity of the compound to a target sequence, enhanced cellular uptake of the compound, increased resistance of the compound to degradation or increased silencing activity of the compound, particularly in vivo. Such modifications are considered to be within the skill of a person in the art with reference to the literature and the description herein. In some embodiments, a dsRNA agent useful in the methods of the invention comprise at least one modified nucleotide, such as a 2′-O-methyl modified nucleotide, a nucleotide comprising a S′-phosphorothioate group, and a terminal nucleotide linked to a cholesterol derivative. Alternatively, the modified nucleotide may be chosen from the group of: a 2′˜deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

### Ribozymes

In one embodiment, an agent useful in the method of the invention is a ribozyme that inhibits the expression of said WTX polypeptide and one or more expression vectors encoding said ribozyme.

In one embodiment, a ribozyme useful in the methods of the invention is a catalytic RNA molecule that cleaves a specific target RNA. A useful ribozyme may be chemically synthesized or may be comprised in a suitable recombinant vector for subsequent introduction and expression in a particular cell, cell-type or tissue. Chemical synthesis and construction of a suitable recombinant vector for expression of ribozymes, in vitro and in vivo, is considered within the skill of those in the art as known in the literature and as described herein.

Generally, ribozymes are RNA-protein complexes that cleave nucleic acids in a site-specific fashion. Ribozymes contain specific catalytic dₒₘains that possess endonuclease activity (Kim and Cech, 1987³⁶; Forster and Symons, 1987³⁷). Ribozymes can be designed as known and used in the art, to target specific nucleotide sequences and provide sequence specific inhibition of gene expression (U.S. Pat. No. 5,354,855). Ribozyme catalysis is part of sequence-specific cleavage/ligation reactions involving nucleic acids and is based on the requirement for specific base-pairing interactions to the internal guide sequence (“IGS”) of a ribozyme with a target sequence prior to ribozyme initiation of target cleavage (Joyce, 1989³⁸; Cech et al., 1981³⁹)

Ribozymes useful as agents in the methods of the invention may be formed as hammerhead, hairpin, a hepatitis δ virus, group I intron or RNaseP RNA (in association with an RNA guide sequence) or Neurospora VS RNA motifs. Useful ribozymes are designed to comprise a specific substrate binding site which is complementary to one or more of the target gene RNA regions, and additional nucleotide sequences within or surrounding that substrate binding site which impart an RNA cleaving activity to the molecule.

In a non-limiting example, ribozymes useful in the present invention include those described in U.S. Pat. No. 5,824,519, the entirety of which is incorporated by reference herein).

Formulations of Antisense Compounds, dsRNAs and Ribozymes

Antisense compounds, dsRNAs, and ribozymes can be formulated as bioequivalent compounds, including pharmaceutically acceptable salts and prodrugs. Such formulation encompasses any pharmaceutically acceptable salts, esters, or salts of such esters, or any other compound which, upon administration to an animal including a human, is capable of providing (directly or indirectly) the biologically active metabolite or residue thereof.

Accordingly, for example, an antisense compound, dsRNA, or ribozyme can be formulated as a pharmaceutically acceptable salt of a nucleic acid and a prodrug of such a nucleic acid. Pharmaceutically acceptable salts retain the desired biological activity of the parent compound and do not impart undesired toxicological effects thereto (see, for example, Berge et al., “Pharmaceutical Salts,” J. of Pharma Sci. 1977, 66, 1-19). Pharmaceutically acceptable salts of antisense oligonucleotides include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

Antisense compounds, dsRNAs, and ribozymes can also be formulated as prodrugs or in prodrug form. A prodrug is a therapeutic agent that is prepared in an inactive form that is converted to an active form (i.e., drug) within the body or cells of a subject by the action of endogenous enzymes or other chemicals and/or conditions. In particular, prodrug versions of antisense oligonucleotides may be prepared as SATE [(S-acetyl-2-thioethyl) phosphate] derivatives according to the methods disclosed in WO 93/24510 to Gosselin et al., published Dec. 9, 1993.

### WTX Polypeptides

In one embodiment, an agent useful in the methods of the invention is a truncated WTX polypeptide of the invention or a fragment or variant thereof or an expression cassette comprising a polynucleotide which encodes a truncated WTX polypeptide of the invention, as previously described herein. The expression cassette can be comprised in a genetic construct wherein a polynucleotide encoding a truncated WTX polypeptide of the invention or fragment or variant thereof is operably linked to a polynucleotide regulatory element that may be a homologous or heterologous promoter. Preferably the promoter is an inducible or regulatable promoter. Preferably the promoter is a homologous promoter. Preferably the promoter is a heterologous promoter.

### Small Molecule Inhibitors

In one embodiment, an agent useful in the methods of the invention is a small molecule inhibitor of WTX gene expression or activity. Exemplary small molecule inhibitors useful in the methods of the invention can be identified as described in U.S. Pat. No. 6,686,148 (the entirety of which is incorporated herein by reference). Additional small molecule inhibitors will be identified by screening of compound libraries as known in the art and described in the literature.

Any compound that falls within the definition of small molecule inhibitor as used herein may be screened as a candidate small molecule inhibitor using the screening methods of the invention as described herein or any other method of screening as known in the art and described in the literature. Examples of compounds that could be screened include inorganic and organic compounds such as, but not limited to, amino acids, peptides, proteins, nucleotides, nucleic acids, glyco-conjugates, oligosaccharides, lipids, alcohols, thiols, aldehydes, alkylators, carbonic ethers, hydrazides, hydrazines, ketones, nitrils, amines, sulfochlorides, triazines, piperizines, sulphonamides and the like.

The term “compound” or “substance” as used herein includes heterogeneous mixtures, such as natural extracts from any organism.

Preferably compound libraries are screened in the methods of the invention. Methods for synthesizing and screening compound libraries are known to those skilled in the art. See for example, U.S. Pat. Nos. 5,463,564; 5,574,656; 5,684,711; and 5,901,069, the entirety of which are herein incorporated by reference. Methods for identifying compounds which bind to polypeptides can be employed as known and described, for example in WO 03/077648.

Candidate agents which can be screened according to the methods of the invention can be obtained using any of the numerous approaches in combinatorial library methods known in the art, including: biological libraries; spatially addressable parallel solid phase or solution phase libraries; synthetic library methods requiring deconvolution; and synthetic library methods using affinity chromatography selection.

Libraries of compounds may be presented in solution, or on beads or chips or the like. Exemplary libraries include, but are not limited to synthetic chemical product libraries and natural product libraries (e.g., partially deconvoluted natural product libraries).

A “candidate agent” as used herein is any agent to be tested in the methods of the invention and may be derived from any source. The term “candidate agent” as used herein is not limited to individual molecules and may include mixtures and extracts. Small molecule inhibitors as described herein may be “candidate agents” that are screened for and identified as agents that are useful in the methods of the invention for inhibiting the expression or activity of a WTX polypeptide.

The candidate agent may include any foreign molecule not usually present in the cell or to which the cell would not normally be exposed during its life cycle. For example, the cell may be exposed to a candidate agent that is a compound or substance listed in a pharmacopoeia with known pharmacological activity. Alternatively, the compound/substance may be one known to interact with a particular biochemical pathway or gene. A further alternative is to test known molecules with no known biological activity or completely new molecules or libraries of molecules such as might be generated by combinatorial chemistry.

Small molecule inhibitors can be formulated for delivery in pharmaceutical compositions as known in the art and as described herein. Administration of a useful small molecule inhibitor can also be carried out as known in the art and as described herein for other agents useful in the methods of the invention.

### Aptamers

In one embodiment, an agent useful in the methods of the invention is an aptamer that inhibits WTX gene expression or activity.

An “aptamer” refers to a nucleic acid ligand that binds to more than one site on a target molecule where binding is not “complementary,” i.e., is not due to base-pair formation between a nucleic acid ligand and a target nucleic acid sequence. An aptamer can be designed which binds to any envisionable target, including large and small biomolecules, such as lipids, carbohydrates and proteins and including proteins not known to bind nucleic acids as part of their biological function and nucleic acid-binding proteins. Aptamers can also be designed that bind other small molecules.

Exemplary aptamers useful in the methods of the invention can be designed following the methods of aptamer design as known in the art and as disclosed in, for example, U.S. Pat. No. 5,773,598, U.S. Pat. No. 5,637,459, and US20060257900A1 (the entirety of each being incorporated herein by reference).

Useful aptamers can be formulated for delivery in pharmaceutical compositions as known in the art and as described herein. Administration of a useful aptamers can also be carried out as known in the art and as described herein for other agents useful in the methods of the invention.

### Formulations and Pharmaceutical Compositions

An agent that is useful in the methods of the invention can be formulated for therapeutic or prophylactic treatments in various ways as known and disclosed in the art. Agents can be formulated for administration in a pharmaceutical composition. Pharmaceutical compositions may include, but are not limited to, pharmaceutically acceptable carriers, proteins, small peptides, salts, excipients, thickeners, diluents, buffers, preservatives, surface active agents, neutral or cationic lipids, lipid complexes, liposomes, penetration enhancers, carrier compounds and other pharmaceutically acceptable carriers and the like in addition to the agent. Such compositions and formulations can be used in accordance with the present invention.

A pharmaceutically acceptable carrier may be liquid or solid and is selected as known in the art, in view of a planned manner of administration. A pharmaceutically acceptable carrier provides for the desired bulk, consistency, etc of a pharmaceutical composition that is to be used or delivered in a particular context.

A pharmaceutically acceptable carrier typically includes binding agents (e.g., pregelatinized maize starch, polyvinylpyrrolidone (PVP) or hydroxypropyl methylcellulose, and the like, fillers such as lactose or other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.).

Penetration enhancers may be included in pharmaceutical compositions in order to enhance the delivery of a useful agent. Examples of penetration enhancers include fatty acids, bile salts, chelating agents, surfactants and non-surfactants (Lee et al., Critical Reviews in Therapeutic Drug Carrier Systems 1991, 8, 91-192; Muranishi, Critical Reviews in Therapeutic Drug Carrier Systems 1990, 7, 1-33), but are not limited thereto. Single penetration enhancers may be used alone or in combination with any other penetration enhancer disclosed herein.

Examples of fatty acids (and derivatives thereof) useful as penetration enhancers include, but are not limited to, oleic acid, lauric acid, capric acid, myristic acid, palmitic acid, stearic acid, linoleic acid, linolenic acid and physiologically acceptable salts thereof.

Suitable chelating agents are well known and disclosed in the art. Such agents include, but are not limited to, disodium ethylenediaminetetraacetate (EDTA), citric acid, salicylates (e.g., sodium salicylate, 5-methoxysalicylate and homovanilate) and N-acyl derivatives of collagen.

Likewise, numerous surfactants are well known and disclosed in the art. Examples include, but are not limited to, sodium lauryl sulfate, polyoxyethylene-9-lauryl ether and polyoxyethylene-20-cetyl ether.

Examples of non-surfactants include, but are not limited to, unsaturated cyclic ureas, 1-alkyl- and 1-alkenylazacyclo-alkanone derivatives and non-steroidal anti-inflammatory agents such as diclofenac sodium, indomethacin and phenylbutazone

The agents useful in the methods of the present invention can be formulated in pharmaceutical compositions that contain additional functional or therapeutic components or delivery reagent. Such other components can be considered adjunct components as may be conventionally found in pharmaceutical compositions, at their art-established usage levels. Examples of such components include compatible pharmaceutically-active materials such as local anesthetics or anti-inflammatory agents. Additional materials useful in physically formulating various dosage forms of a pharmaceutical composition may also be included, such as dyes, flavoring agents, preservatives, antioxidants, opacifiers, thickening agents and stabilizers.

In one embodiment, an agent useful in the methods of the invention is formulated with or conjugated to a delivery reagent. A delivery agent may be a molecule, molecular structure or mixture of molecules or compounds that is admixed, used to encapsulate, conjugated to or otherwise associated with an agent that is used in the methods of the invention. Exemplary delivery reagents include, but are not limited to, antibodies or fragments or variants thereof that are capable of binding, or that are capable of specifically binding a WTX polypeptide of the invention.

In one embodiment, a delivery agent may be a cell-penetrating peptide as known or used in the art. Peptide vectors have been used to deliver various macromolecules into cells (across plasma membranes), including delivery of proteins and nucleic acids. Exemplary cell-penetrating peptides include, but are not limited to, MPG, a short peptide vector (US2002/0009758), Penetratin-1, a 16-amino-acid polypeptide derived from the third alpha-helix of the homeodomain of Drosophila antennapedia (US2005/0260756 and US2006/0178297), biotin (U.S. Pat. No. 6,287,792), transportan (U.S. Pat. No. 6,025,140; Pooga, et al., 1998, FASEB J., 12(1), 67-77), pISI, (Magzoub, et al., 2001, Biochim. Biophys. Acta, 1512(1), 77-89; Kilk, et al., 2001, Bioconjug. Chem., 12(6), 9 11-916), Tat(48-60) Vives, et al., 1997, J. Biol. Chem., 272(25), 16010-16017), pVEC (Elmquist et al., 2001, Exp. Cell Res., 269(2), 237-244), MAP (Scheller, et al., 1999, J. Peptide Science, 5(4), 185-194; Hallbrink et al., 2001, Biochim Biophys Acta, 1515(2), 101-109) and MTS (Lindgren et al., 2000, Trends in Pharmacological Sciences, 21(3), 99-103).

Cell-penetrating peptides and dsRNAs may be operably linked to form a cell-permeable complex. Cell-penetrating peptides and dsRNAs are each prepared separately and modified or derivatized with reactive groups appropriate to allow for linkage. The modified components (the cell-penetrating peptide and the dsRNA) are then incubated together, for a sufficient time and under appropriate conditions of temperature, pH, molar ratio, etc., to allow covalent bond formation between the reactive groups of each modified component. Various methods and protocols for conjugation, including the specific conditions for efficient conjugation, are within the skill of a person in the art and described in the literature.

Cell-penetrating peptides/dsRNA complexes are suitable for in vivo delivery of useful dsRNA agents in the methods of the invention and may be used advantageously in protocols involving the translocation of macromolecules into cells, including non-traumatic internalization, limited endosomal degradation, high translocation efficiencies at low concentrations, and delivery to a wide variety of cell and tissue types. In some embodiments, “cell-penetrating peptides” are used to deliver dsRNA, antisense compounds or ribozymes useful in the methods of the invention and may provide a greater efficiency of transport across cell membranes as compared to conventional methods known in the art, such as transfection, electroporation, liposomal delivery, or microinjection. In some embodiments, cell-penetrating peptides may also be conjugated to liposomes or other delivery agents as described herein, and may be used to aid in the delivery of any delivery agent/useful agent complex within to the methods of the invention.

In one embodiment, by facilitating the transport of a dsRNA or siRNA into cells, the dsRNA or siRNA/“cell-penetrating peptide” complex may provide a means to temporarily decrease the expression of genes targeted by the dsRNA or siRNA incorporated in the complex (US2005/0260756). The dsRNA or siRNA incorporated in the complex is designed target a particular mRNA in a cell, allowing selective inhibition of a particular mRNA transcript.

Additional delivery agents include, but are not limited to, various targeting ligands as known and used in the art, such as folic acid, polyethylene glycols, carbohydrate clusters, cross-linking agents and porphyrin conjugates.

In one embodiment, colloidal dispersion systems may be used as delivery vehicles to enhance the in vivo stability of the useful agent. A colloidal dispersion system can also be used to provide a timed and/or transient release of the useful agent. Release of the agent can be by continuous release at a prescribed rate or can be by pulsed release of specified amounts at pre-determined dosage intervals. Such timed and/or transient release is comprehended by the art and will be formulated with particular regard for the agent to be delivered as is art-understood, in accordance with known and used methods, to provide the desired amounts or to maintain the desired levels of the agent.

Preferably the delivery vehicle targets or maintains the agent to or at a particular organ, tissue or cell type. Exemplary colloidal dispersion systems include, but are not limited to, macromolecule complexes, microcapsules, nanocapsules, microspheres, bioadhesive microspheres, nanospheres, biodegradable nanospheres, beads, microbeads, gels and hydrogels and lipid-based systems including oil-in-water emulsions, slurries, gels, pastes, micelles, mixed micelles and liposomes.

As known in the art and used herein, microparticles, microspheres, microcapsules and nanoparticles refers to a particle, which is typically a solid, containing the active agent to be delivered. The active agent is comprised within the core of the particle or may be attached to a polymer that comprises or covers the particle. Microparticles (or microcapsules or microspheres) and nanoparticles generally differ in size. Microparticles typically have a particle size range of about 1 to about 1000 microns. Nanoparticles typically have a particle size range of about 10 to about 1000 nm.

Liposomes in a carrier delivery system can be liposomes comprising a single agent useful in the methods of the invention or a plurality of liposomes comprising a plurality of agents useful in the methods of the invention. Liposomes are microscopic spheres having an aqueous core surrounded by one or more outer layers made up of lipids arranged in a bi-layer configuration (see, generally, Chonn et al., Current Op. Biotech. 1995, 6, 698-708). As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which may be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids such as cholesterol; cerebrosides; sphingomyelin; glycerolipids; and other lipids (see for example, U.S. Pat. No. 5,833,948). Suitable liposomes which may be employed to deliver agents useful in the methods of the invention include, but are not limited to, fusogenic liposomes, immunoliposomes and PEG conjugated liposomes, but not limited thereto.

Hydrogels, as known in the art and used herein, are typically cross-linked, hydrophilic polymer networks that are permeable to a wide variety of drug compounds. Advantageously, polymer swelling of a hydrogel may be selectively triggered, yielding a controlled release of an entrapped compound. Depending on the composition of the polymer network, the particular conditions that will elicit selective swelling and subsequent release of a compound from a hydrogel will depend on the specific composition of the polymers comprised in of the hydrogel network. An appropriate hydrogel can be chosen which will respond to a variety of trigger stimuli including pH, ionic strength, thermal, electrical, ultrasound, and enzyme activities. Non-limiting examples of polymers useful in hydrogel compositions include hydrogels formed from polymers of poly(lactide-co-glycolide), poly(N-isopropylacrylamide); poly(methacrylic acid-g-polyethylene glycol); polyacrylic acid and poly(oxypropylene-co-oxyethylene) glycol; and natural compounds such as; chrondroitan sulfate, chitosan, gelatin, or mixtures of synthetic and natural polymers, for example chitosan-poly(ethylene oxide). The polymers may be cross-linked reversibly or irreversibly to form gels in which an agent useful in the methods of the invention can be embedded (see for example, U.S. Pat. Nos. 6,451,346; 6,410,645; 6,432,440; 6,395,299; 6,361,797; 6,333,194; 6,297,337 and Johnson, O. et al., Nature Med. 2: 795 (1996); the entirety of each is incorporated herein by reference).

The agents useful in the methods of the present invention may be administered in a number of ways depending upon whether local or systemic treatment is desired and upon the area to be treated. Administration may be topical (including intranasal, epidermal, and transdermal), oral or parenteral. Parenteral administration includes direct application, systemic, subcutaneous, intraperitoneal or intramuscular injection, intravenous drip or infusion, inhalation, insufflation or intrathecal or intraventricular administration.

Useful agents may be formulated for parenteral administration in any appropriate solution, including sterile aqueous solutions which may also contain buffers, diluents and other suitable additives

Useful agents may be formulated for oral administration in powders or granules, aqueous or non-aqueous suspensions or solutions, capsules, pills, lozenges or tablets. Thickeners, flavoring agents, diluents, emulsifiers, dispersing aids or binders may be desirable.

Useful agents may be formulated for topical or direct administration in transdermal patches, subdermal implants, ointments, lotions, creams, gels, drops, pastes, suppositories, sprays, liquids and powders. Conventional pharmaceutical carriers, aqueous, powder or oily bases, thickeners and the like may be necessary or desirable.

A person skilled in the art will be able to choose the appropriate mode of administration of an agent useful in the methods of the invention with reference to the literature and as described herein. By way of non-limiting example, a systemic application would be preferred for the treatment of osteoporosis whereas a local application would be preferred for the repair or reconstruction of bone trauma or injury, for example, for implant and prosthesis support or fracture healing, but not limited thereto. Additionally, administration via implant coating may also be employed for implant and prosthesis support. See, for example, U.S. Pat. Nos. 6,716,883; 6,620,406; 4,446,578; 6,428,803 and 5,069,905 (the entirety of each is incorporated herein by reference.

In a preferred embodiment, direct application comprises the direct application, at or near the site of bone trauma, reconstruction, fracture, breakage or graft, of a of a liquid (including a colloidal dispersion), a paste, a gel, a slurry, or is via a patch, a wrap, a plug, an implant comprising a porous material, an implant comprising a non-porous material, or an implant comprising a gelatinous material, a coated implant or by iontophoresis.

Preferably the direct application is local application. Preferably local application includes application of an agent useful in the methods of the invention in combination with a delivery reagent or additional therapeutic agent that serves to retain the useful agent in a particular cell or tissue or within a particular region of the body of a subject, e.g. a region of an arm, leg, neck, head and the like, but not limited thereto.

Preferably administration to a subject, of an agent useful in the methods of the invention is transient administration. Preferably transient administration comprises administration of a useful agent for a sufficient period of time to provide a treatment or achieve a therapeutic result without the presence of the useful agent resulting in an undesired or harmful effect. Administration can be rapid (e.g., by injection), or can occur over a period of time (e.g., by slow infusion or administration of slow release formulations).

Any agent useful in the methods of the invention can be formulated as bioequivalent compounds, including pharmaceutically acceptable salts and prodrugs. Such formulation encompasses any pharmaceutically acceptable salts, esters, or salts of such esters, or any other compound which, upon administration to an animal including a human, is capable of providing (directly or indirectly) the biologically active metabolite or residue thereof.

Pharmaceutically acceptable salts retain the desired biological activity of the parent compound and do not impart undesired toxicological effects thereto (see, for example, Berge et al., “Pharmaceutical Salts,” J. of Pharma Sci. 1977, 66, 1-19). Pharmaceutically acceptable salts of some useful agents include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

Agents can also be formulated as prodrugs or in prodrug form. A prodrug is a therapeutic agent that is prepared in an inactive form that is converted to an active form (i.e., drug) within the body or cells of a subject by the action of endogenous enzymes or other chemicals and/or conditions.

The formulation of pharmaceutical and therapeutic compositions and their subsequent administration is believed to be within the skill of those in the art. A particular and effective dosage regime will be dependent on severity of the disease or condition to be treated and on the responsiveness of the treated subject to the course of treatment. An effective treatment may last from several days to several months, or until an acceptable therapeutic outcome is effected or assured or until an acceptable reduction of the disease or condition is observed. An optimal dosing schedule (s) may be calculated from drug accumulation as measured in the body of a treated subject. It is believed to be within the skill of persons in the art to be able to easily determine optimum and/or suitable dosages, dosage formulations and dosage regimes. Of course, the optimum dosages may vary depending on the relative potency of a given agent but will be estimable from an EC50s found to be effective in suitable cells in vitro and in an appropriate in vivo animal model. In general, dosage is from 0.001·g to 99 g per kg of body weight, and may be given once or more daily, weekly, monthly or yearly, or even once every 20 years.

In one embodiment, the agent useful in the methods of the invention is administered in conjunction with the administration of an additional therapeutic agent. The additional therapeutic agent can be any appropriate therapeutic agent used to treat or prevent any symptom, side effect or other consequence of treatment, either as a result of the use of an agent used in the methods of the invention or for any other reason related to the desired treatment. In a preferred embodiment, the additional therapeutic agent is active in bone cells and/or is used to treat a symptom or cause of a bone-related disease or condition.

Preferably, the agent useful in the methods of the invention is administered in combination with one or more bone regeneration-promoting cofactors including, but not limited to, bone morphogenic factors designated BMP-1 through BMP-12, TGF-, and TGF-, B family members, FGF-1 to FGF-10, interleukin-1 inhibitors, TNFα inhibitors, parathyroid hormone and analogs thereof, parathyroid related protein and analogs thereof, E series prostaglandins, bisphosphonates (such as alendronate and others), vitamin D3, dexamethasone, and bone-enhancing minerals such as fluoride and calcium.

Where an agent useful in the methods of the invention is formulated with an additional therapeutic agent, then the dosing of the useful agent and the additional therapeutic agent can be separate, simultaneous or sequential, as is appropriate. Repetition rates for dosing can be based on measured residence times and concentrations of a given agent in the cells, fluids or tissues of the subject that is being or that is to be treated. Maintenance therapy may be desirable in successfully treated patient in order to prevent the recurrence of the disease or condition, wherein the useful agent is administered in maintenance doses, ranging from 0.001·g to 99 g per kg of body weight, once or more daily, to once every 20 years.

A further aspect of the invention provides a method for identifying an agent which promotes bone healing or bone formation, the method comprising screening one or more candidate agents for the ability to modulate the expression or activity of a WTX polypeptide, wherein the candidate agent that modulates expression or activity of a WTX polypeptide is an agent that promotes bone healing or bone formation.

In another aspect, the invention provides a method for identifying an agent which modulates osteoprogenitor cell differentiation, the method comprising contacting an osteoprogenitor cell with one or more candidate agents and assaying the contacted osteoprogenitor cells for modulated expression or activity of a WTX polypeptide as compared to control osteoprogenitor cells that are not contacted with said one or more candidate agents, wherein the candidate agent that modulates expression or activity of a WTX polypeptide is an agent that modulates osteoprogenitor cell differentiation.

In a further aspect, the invention provides a method for identifying an agent which increases or stimulates osteoblast differentiation, the method comprising contacting a multipotent progenitor cells stem cell with one or more candidate agents and assaying the contacted cells for modulated expression or activity of a WTX polypeptide as compared to control multipotent progenitor cells that are not contacted with said one or more candidate agents, wherein the candidate agent that modulates expression or activity of a WTX polypeptide is an agent that increases or stimulates osteoblast differentiation.

In still another aspect, the invention provides a method for identifying an agent which stimulates or enhances mineralization by a cell, the method comprising contacting a cell with one or more candidate agents and assaying the contacted cells for modulated expression or activity of a WTX polypeptide as compared to control cells that are not contacted with said one or more candidate agents, wherein the candidate agent that modulates expression or activity of a WTX polypeptide is an agent that stimulates or enhances mineralization by a cell:

In one embodiment the agent used in the above methods of screening is any candidate agent that modulates the expression or activity of a WTX polypeptide. In a preferred embodiment, the agent is selected from the group consisting of:
(i) an antibody capable of specifically binding to a WTX polypeptide,
(ii) an antisense compound that inhibits the expression of said WTX polypeptide,
(iii) an expression vector encoding said antisense compound,
(iv) a dsRNA that inhibits the expression of said WTX polypeptide,
(v) one or more expression vectors encoding said dsRNA,
(vi) a ribozyme that inhibits the expression of said WTX polypeptide,
(vii) one or more expression vectors encoding said ribozyme,
(viii) a fragment of a WTX polypeptide,
(ix) a truncated WTX polypeptide of the invention,
(x) an expression vector comprising a polynucleotide which encodes a polypeptide of the invention,
(xi) a fusion protein comprising a truncated WTX polypeptide of the invention,
(xii) a small molecule inhibitor of WTX gene expression or activity,
(xiii) an aptamer that inhibits WTX gene expression or activity, and
(xiv) a peptide nucleic acid that inhibits WTX gene expression or activity.

Preferably screening one or more candidate agents involves measuring in increase or decrease in a WTX polynucleotide or polypeptide expression in vitro or in a cell ex vivo.

Preferably screening one or more candidate agents involves measuring in increase or decrease in a WTX polynucleotide or polypeptide expression in a cell in vivo. The cell in vivo may be in any organism, preferably a non-human organism, more preferably a non-human animal.

Preferably assaying for modulated expression or activity of a WTX polypeptide involves measuring the levels of expression of a WTX polynucleotide or measuring the amount of a WTX polypeptide in a cell as compared to a control cell.

Preferably assaying for modulated activity of a WTX polypeptide involves measuring the levels of binding of a WTX polypeptide to one or more candidate agents. Preferably the level of binding is determined in a cell or in vitro.

Preferably assaying for modulated activity of a WTX polypeptide involves measuring the levels of binding of a WTX polypeptide to another polypeptide to which WTX normally binds. Preferably the level of binding is determined in a cell or in vitro.

Preferably assaying for modulated activity of a WTX polypeptide involves measuring the levels of expression of genes or polypeptides in a WTX associated cell signaling pathway induced by WTX activity.

Preferably assaying for modulated activity of a WTX polypeptide involves measuring the levels of expression of genes or polypeptides involved in osteoprogenitor cell differentiation induced by WTX activity.

Preferably assaying for modulated activity of a WTX polypeptide involves determining the levels of expression of genes or polypeptides involved in stimulated or enhanced mineralization in a cell, more preferably in a bone cell, most preferably in an osteoblast cell induced by WTX activity.

Methods of identifying candidate agents according to the screening methods of the invention employ standard screening techniques and protocols that are known in the arts of cell and molecular biology and used routinely by those of ordinary skill in the art and as previously described herein (Sambrook et al. supra).

In one embodiment, the screening methods of the invention assay the ability of a candidate compound to modulate the expression or activity of a WTX polypeptide. Expression or activity of a WTX polypeptide can be assayed routinely as known in the art and previously described herein, for example, by antibody binding to a WTX polypeptide, by measurement of the levels of transcription of a WTX mRNA, by measurement of a the levels of translation of a WTX polypeptide and the like, but not limited thereto (Sambrook et al. supra).

Preferably each of the assay methods described above includes a step comprising a chemical or molecular method of assaying for modulation or alteration of a WTX polypeptide expression or activity. Such a step can be combined with additional steps that measure additional modulation or alteration of a WTX associated cell signaling pathway or other phenotypic indicators or both wherein the modulation or alteration of the WTX associated cell signaling pathway or other phenotypic indicator is identified as being induced by WTX expression or activity.

In one embodiment, the screening methods of the invention include a further step of assaying for phenotypic changes in contacted cells as compared to non-contacted cells, including, but not limited to, cell differentiation, cell growth and cell shape wherein the phenotypic changes are identified as being induced by WTX expression or activity.

Phenotypic changes can be morphological changes observed by eye or microscopically as known in the art and described herein. Preferably cell differentiation is measured in osteoprogenitor cells or multipotent progenitor cells by determination of the ratio of osteoblast cells to chondroblast or adipocyte cells in response to contact with a candidate compound, as compared to non-contacted osteoprogenitor cells or multipotent progenitor cells.

In non-limiting embodiment, osteoprogenitor, multipotent progenitor or osteoblast cell populations are used to assay for agents that modulate cell differentiation via the modulation or alteration of WTX gene or polypeptide expression or activity. Preferably the cell populations are derived from a human. Compounds so identified are particularly useful for the development of drugs which directly act on fat tissue in vivo.

Preferably, osteoprogenitor, multipotent progenitor or osteoblast cells are contacted with a candidate agent in the presence of differentiation medium and cultured for a predetermined amount of time in the presence of the candidate agent and the differentiation medium. The predetermined amount of time is generally an amount of time sufficient to allow differentiation of cells into osteoblasts. The differentiated cells are then assayed at the end of the predetermined time for modulation of WTX gene or polypeptide expression or activity.

In another non-limiting embodiment, a bone-related cell, preferably a bone cell, is contacted with a candidate compound and, in addition to the step of assaying for WTX polypeptide expression or activity, the extent of mineralization by the cell is determined under a particular set of conditions as being induced by WTX expression or activity. Preferably the cell is an osteoprogenitor cell, a multipotent progenitor cell or an osteoblast cell.

Mineralization assay conditions can be in vitro or in vivo conditions. The extent of mineralization by the cell is then compared to the extent of mineralization by a non-contacted control cell of the same type and under the same particular conditions. An increased amount of mineralization or rate of mineralization by the contacted cell as compared to the non-contacted cell is considered enhanced or stimulated mineralization.

Preferably, bone cells are contacted with a candidate agent in the presence of mineralization medium and cultured for a predetermined amount of time in the presence of the candidate agent and the mineralization medium. The predetermined amount of time is generally an amount of time sufficient to allow mineralization by the contacted cells. The contacted cells are then assayed at the end of the predetermined time for modulation of WTX gene or polypeptide expression or activity.

The extent of mineralization of may be determined by any means as known in the art and described in the literature including, but not limited to, microscopic examination or chemical or molecular measures. Preferably the extent of mineralization is determined by measurement of mineralization itself, for example, by measuring calcium phosphate accumulation, but not limited thereto. Suitable techniques in microscopic examination which can be employed herein include compound light microscopy, interference microscopy, differential interference contrast microscopy, epi-fluorescence microscopy, confocal laser scanning microscopy, micro computed tomographic scanning, scanning electron microscopy or transmission electron microscopy, but are not limited thereto.

Candidate agents that that result in an increase in osteoblast differentiation or increased mineralization by bone cells, as compared to non-contacted cells, may be chosen as selected as potential lead compounds for further investigation.

Numerous methods of screening candidate agents may be employed in the methods of screening cells for modulation or alteration of WTX expression or activity as described herein. Preferably bone cells or bone-related cells, for example, bone-cell progenitors or mesenchymal stem cells, are used in drug screening efforts to identify compounds that act directly on bone or bone-related cells. In one embodiment, rapid high-throughput screening assays or methods are used for identifying compounds or agents that effect or modulate the differentiation of bone related cells. Preferably, high-throughput screening assays are used to identify compounds that modulate the expression or activity of a WTX gene or polypeptide from amongst candidate agents as described herein or as known in the art. Preferably, high throughput assays are performed in 96-well or 384-well assay plates, although other configurations (e.g., other multi-well formats or coverslip formats) are considered to be within the skill of a person in the art.

Lead compounds identified by the methods of screening candidate agents as described herein can further be subjected to a variety of secondary screening assays. In certain embodiments, secondary screening assays are utilized to validate a hit or lead as a modulator of bone-related cell or bone cell growth or differentiation or both. In other embodiments, secondary screening assays are utilized to determine pathways affected by the lead compound or to identify a cell or molecular target of the lead compound, or both, but not limited thereto.

Target identification allows rational design of agents and drugs useful for promoting bone growth, bone density, bone formation and/or bone healing. Identification of an agent and its corresponding target allows identification of specific target sites or motifs required for effect, facilitating the design of more effective drugs.

Candidate agents may be screened for both toxicity and biological activity, e.g., in vitro screening. Candidate agents that are identified in vitro to be non-toxic (e.g., non-toxic at the at a low micromolar level) (potency (ED50)) and biologically active, (e.g., efficacy (maximum response)) are preferred candidate agents. Toxicity screens can be performed utilizing any appropriate cell from a subject, including, for example, bone-related cells, bone cells and non-bone and bone-related cells (e.g., fibroblasts or hepatocytes) but not limited thereto. Toxicity screening may be performed prior to biological activity assays, subsequent to biological activity assays, or in parallel with biological activity assays (i.e., in parallel cultures).

Preferably, toxicity screens of candidate agents are performed in the same cultures as biological activity screens where an acceptable toxicity of the candidate agent is generally less than 20% in the cell assay, more preferably less than 15%, most preferably the toxicity is less than 10%.

Desirable candidate agents will exhibit biological activity detectable as an effect of bone cell replication, differentiation or function, including but not limited to matrix related effects and mineralization by the cell. Preferably, candidate agents will be desirable candidate agents that show a measurable or appreciable effect on replication, differentiation or function while being non-toxic to the assayed cells. Suitable candidate agents may exhibit a potency of at least 500 nm, preferably at least 200 nM, more preferably at least 100 nM, more preferable at least 75 nM, even more preferably at least 50 nM, and even more preferably at least 10 nM. Suitable candidate agents may exhibit at least a 1-2 fold modulation of WTX gene or polypeptide expression or activity as compared to non-contacted cells, preferably at least 3-4-fold, preferably at least 5-6-fold, more preferably at least 7-, 8-, 9- or 10-fold modulation. Preferably modulation is a decrease in WTX gene or polypeptide expression or activity.

Suitable candidate agents may also exhibit at least a 1-2 fold modulation in a measured phenotypic indicator, for example osteoblast differentiation or mineralization, but not limited thereto. Preferably the modulation of the phenotypic indicator will be at least 3-4-fold, preferably at least 5-6-fold, more preferably at least 7-, 8-, 9- or 10-fold modulation. Preferably the modulation is an increase in osteoblast differentiation or matrix mineralization by the cell.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents; or such sources of information, in any jurisdiction, are prior art, or form part of the common general knowledge in the art.

The invention will now be illustrated in a non-limiting way be reference to the following examples.

### EXAMPLES

### Ethical Review, Consent and Patient Ascertainment

All subjects were ascertained by physician-initiated referral and consented to participate under an approved institutional protocol. All individuals had a normal karyotype. Control cell lines were obtained from sex and age-matched healthy individuals.

### Example 1

### Mutations in WTX Lead to X-Linked Bone Disease

### Materials and Methods

### Genomic Analysis and Mutation Detection

Genomic DNA was extracted from blood by standard procedures and applied to the Affymetrix StyI and NspI array at the Australian Genome Research Facility, Melbourne. Copy number analysis utilized the CNAG algorithm₃. The exon and intron-exon boundaries of WTX were amplified by PCR using previously published primers₁₀ and subject to sequencing on an ABI3100 capillary sequencer. Primers for qPCR were designed at three sites over the WTX ORF in addition to two flanking loci. Results were normalised to two autosomal loci, CFTR and SLC16A2. Genomic copy number was calculated by the 2(Delta Delta C(T)) method. Declared parentage was confirmed as consistent for all de novo mutations by the examination of four unlinked microsatellite markers.

### X Inactivation Analysis

Skewing of X-inactivation was measured using a modified HUMARA assay22. Genomic DNA (1 μg) was predigested with RsaI, either in the presence (+) or absence (−) of the methylation sensitive enzyme HpaII. The products were analyzed on an ABI 377 sequencer using GeneScan software. Differences in peak areas for the two alleles in the HpaII (+) assay were corrected for differences in amplification efficiency measured in the HpaII (−) assay, the final results being expressed as a percentage. All experiments were repeated in triplicate. Cellular transformation, expression and co-immunoprecipation HEK293T cells were grown in DMEM supplemented with 10% fetal bovine serum at 37° C. in 5% CO₂. Expression constructs were transiently transfected in HEK293T cells with LF2000 (Invitrogen).

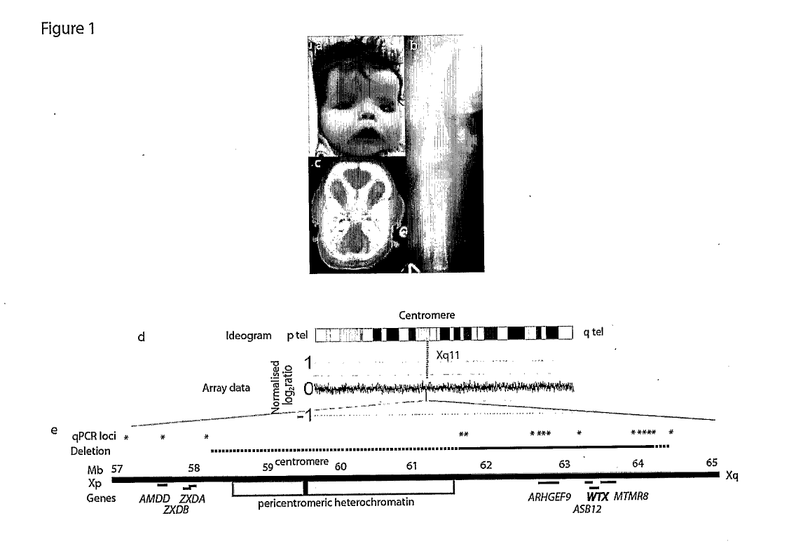

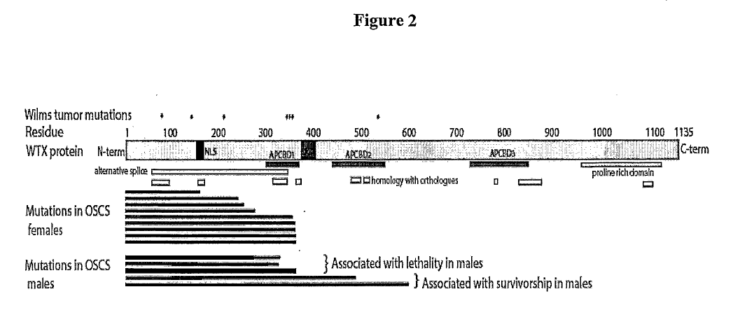

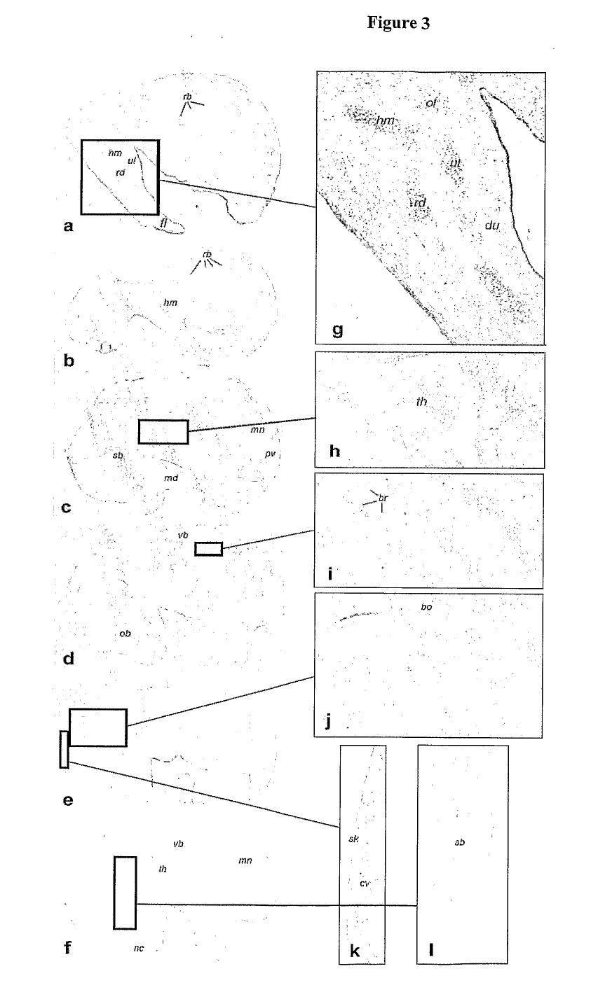

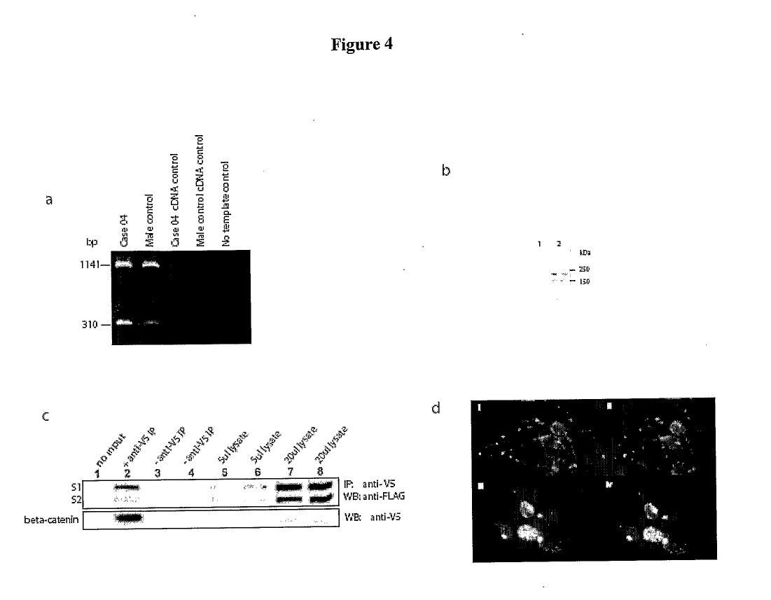

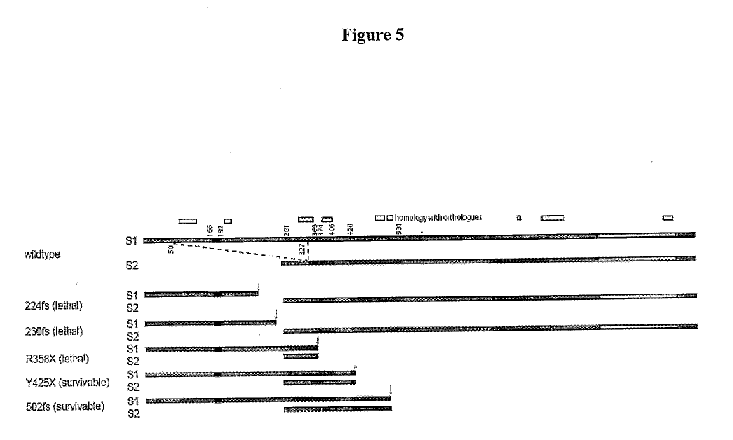

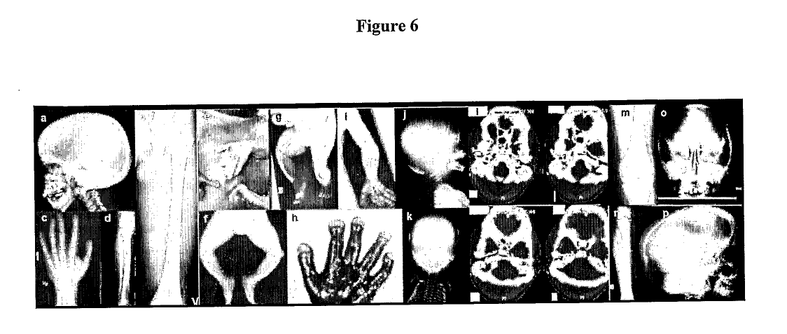

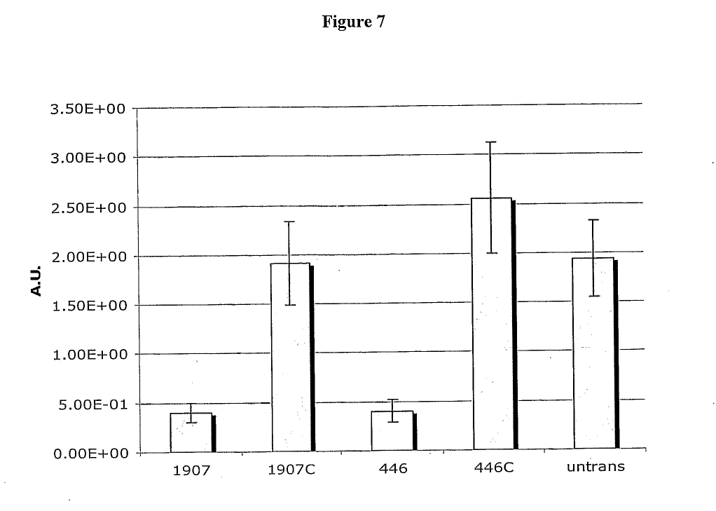

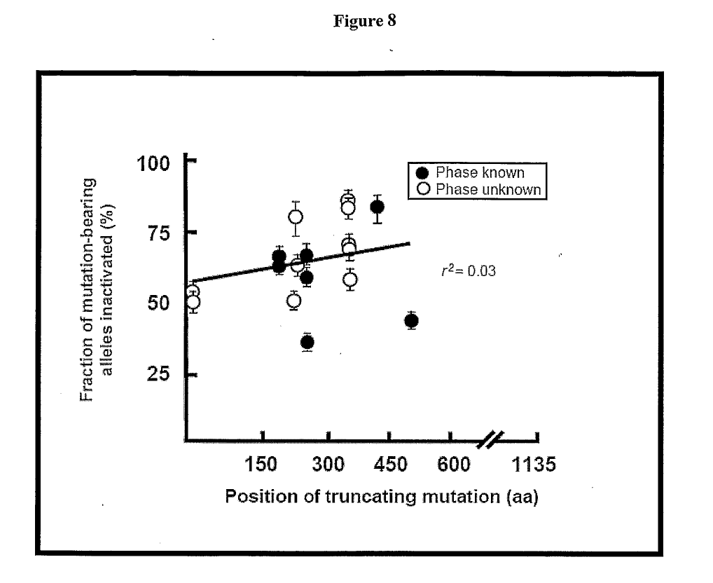

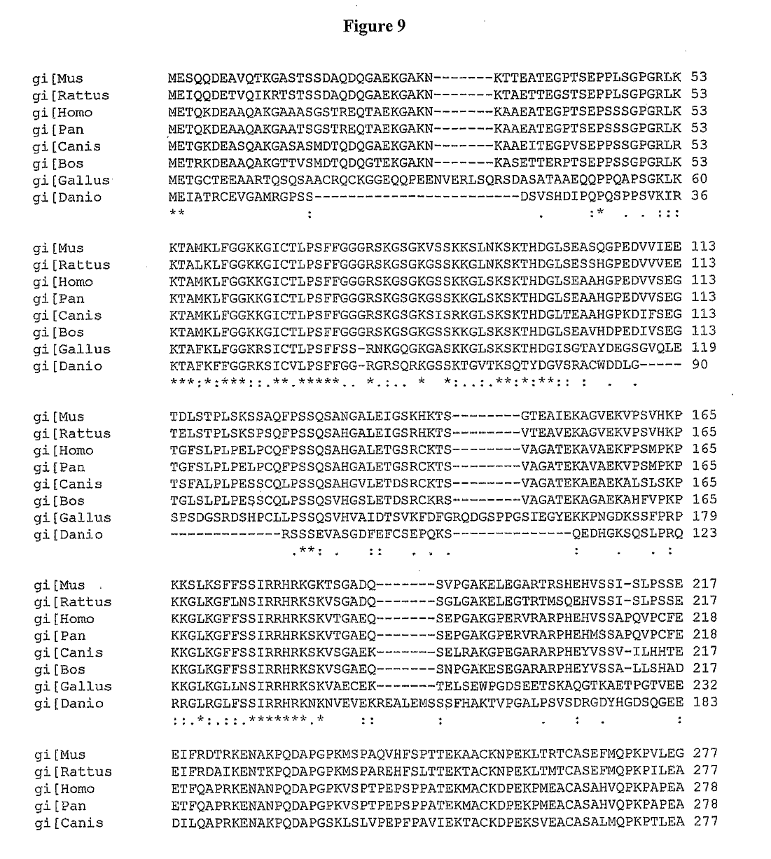

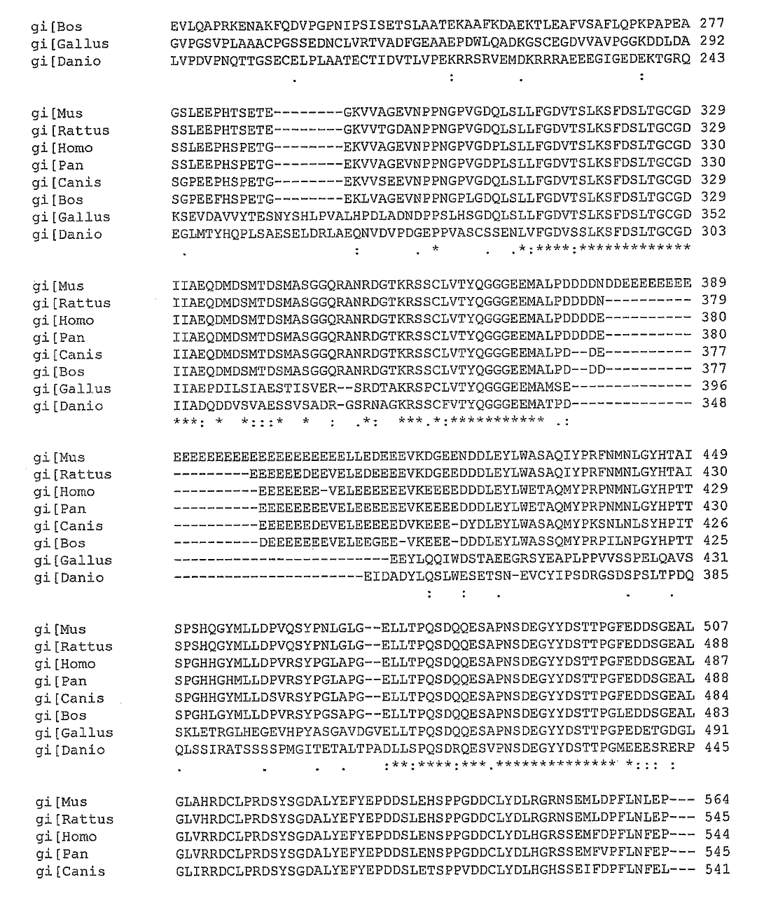

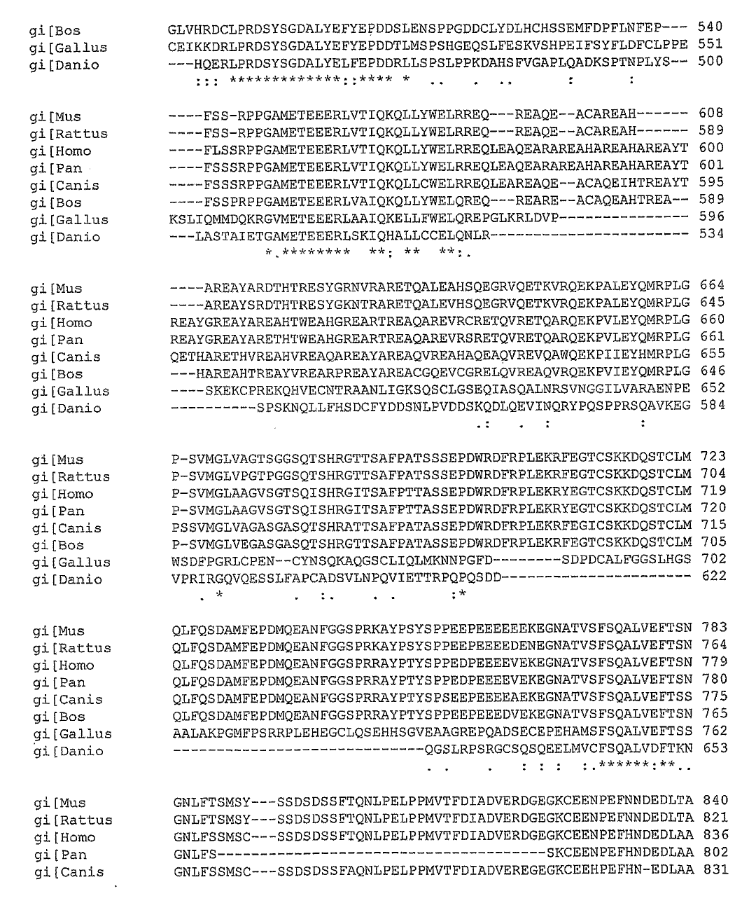

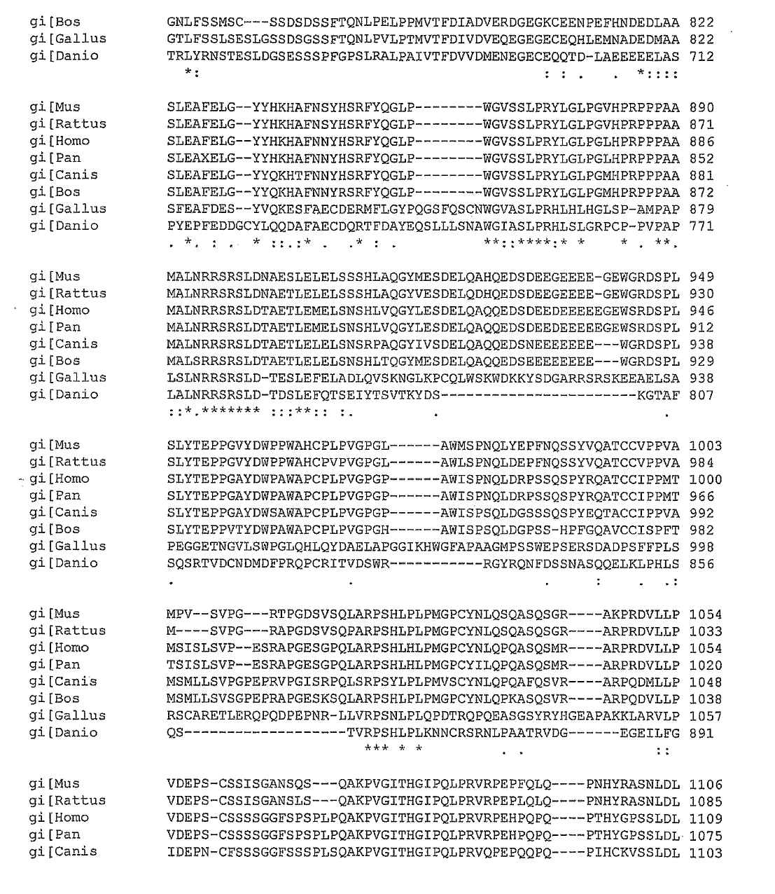

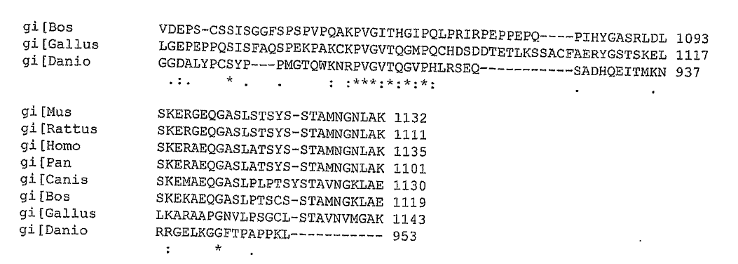

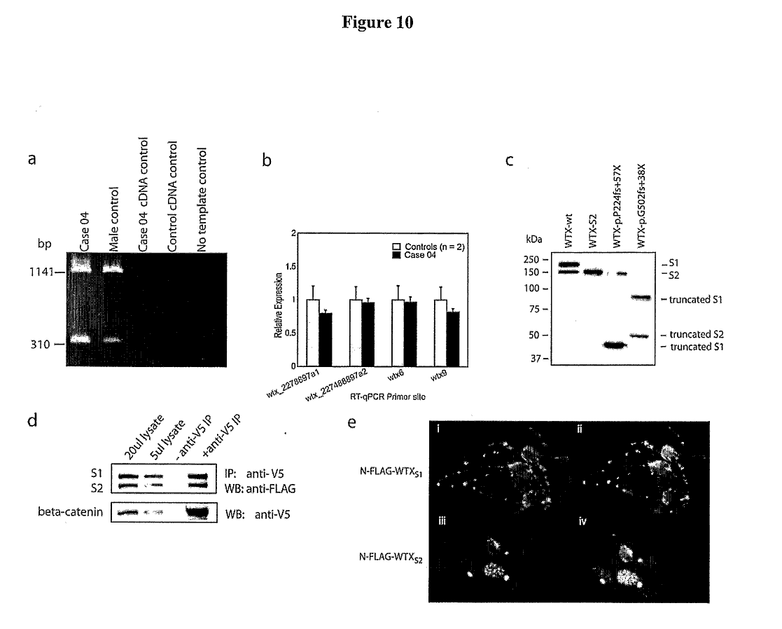

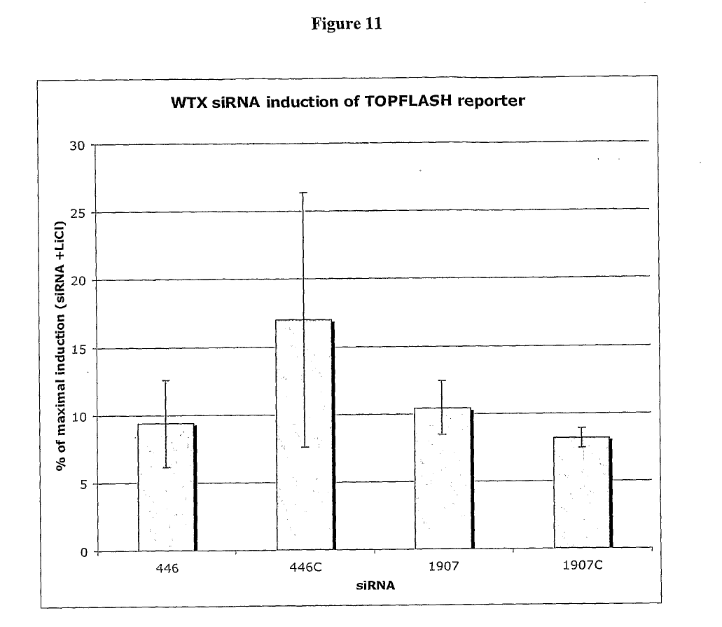

The applicant ascertained a female proband with severe hyperostosis of the skull and appendicular skeleton, severe mental retardation and seizures (FIG. 1a). Her presentation was consistent with X-linked OSCS, an entity usually associated with fetal or neonatal lethality in males. Females with OSCS present with macrocephaly, cleft palate, mild learning difficulties, sclerosis of the long bones and skull and longitudinal striations visible on radiographs of the long bones, pelvis and scapulae (FIG. S1). Occasional surviving males have, in addition to hyperostosis, severe cardiac, intestinal and genitourinary malformations (FIG. S1)2.

Genomic DNA obtained from the proband was analyzed on the Affymetrix 500K SNP array platform and genome-wide copy number assessed using the CNAG algorithm3. A >1.26 Mb deletion at Xq11.1 extending from rs34355157 near the border with pericentromeric heterochromatin to rs41326649 within proximal Xq11 (FIG. 1b,c) was detected and shown to have arisen de novo by qPCR. Of the 4 genes annotated within the deleted region (UCSC Genome Build ver36.2), The applicants considered WTX (Wilms Tumour mutated on the X; FAM123B; AMER1) as a potential monogenic contributor to the skeletal and craniofacial phenotype in this child.

Phenotypic data and DNA samples were subsequently obtained from 17 probands and their affected relatives with OSCS (5 male, 18 female; 14 familial, 9 sporadic; Table 1). Of the five males, three were mid-gestation fetuses with multiple malformations (omphalocele, limb patterning defects, cardiac and genitourinary anomalies) incompatible with survivorship beyond the neonatal period (FIG. S1; Supplementary Table S1). Two surviving males (aged 17 yrs and 23 yrs) had, in addition to a sclerosing skeletal dysplasia, learning disability, cardiomyopathy, skeletal myopathy and central nervous system malformations (FIG. S1; Table S1). Sequencing WTX demonstrated mutations in 16/17 unrelated families. All mutations either deleted the entire gene (n=2) or predicted premature termination of translation (Table 1, FIG. 2). Mutations segregated with the phenotype in all familial cases. Eight mutations arose de novo; the remainder were absent from dbSNP and a panel of 120 control X chromosomes. Two mutations, 1072C>T (R358X) and whole gene deletions were recurrently identified and have previously been reported as somatic mutations in Wilms tumors^{10,13}. All point mutations identified clustered in the 5′region of the WTX (FIG. 2). Mutations leading to lethality in males (n=3) were located more 5′ than those associated with survivability (n=2).

With the above analysis, the applicants have demonstrated that germline mutations in WTX in males (n=5) and females (n=20) lead to the X-linked sclerosing bone dysplasia, osteopathia striata congenita with cranial sclerosis (OSCS; OMIM 1665001), which is characterised by increased bone density and craniofacial malformations.

### Example 2

### WTX is Expressed Nearly Exclusively in the Skeleton in the Midgestation Mouse Embryo

### Materials and Methods

### In Situ Hybridization

Mouse embryos (C57BL6) were collected at E14.5 under an approved animal research protocol. Tissue preparation, automated non-radioactive RNA in situ hybridization with an antisense WTX riboprobe and a sense control riboprobe and digital imaging were performed as described previously¹². To generate antisense and sense riboprobes total RNA from mouse embryos was reverse-transcribed into cDNA using Superscript reverse transcriptase (Clontech, Mountain View, Calif.) and random hexamers (Sigma). PCR amplification was performed using T7-tagged forward and SP6-tagged reverse primers to amplify a 652 bp fragment representing bases 193-845 of the coding region of the murine WTX gene (NM_{—}175179). The PCR-amplified cDNA template was used for in vitro transcription of digoxygenin labeled riboprobe using T7 RNA polymerase for the antisense probe and SP6 for the sense control probe.

The applicants used in situ hybridization to study the endogenous expression pattern of WTX in E14.5dpc murine embryos (FIG. 3). Results of this study detected pronounced expression of WTX in the developing skeleton and skull and also within the pulmonary bronchioles and the thymus. Staining was markedly reduced in the diaphyses of long bones where ossification begins to establish at this gestation (FIG. 3g). As suggested by the OSCS phenotype, these results indicate that WTX mediates its suppressive effects on the ossification of bones that develop both via a cartilage scaffold (e.g. long bones) and by direct differentiation from mesenchymal precursor cells (e.g. calvarium).

With these results, the applicants have shown that WTX is highly expressed in the fetal murine skeleton.

### Example 3

### Identification of Alternative WTX Splice Isoforms

### Materials and Methods

RT qPCR

Total RNA from lymphoblastoid cell lines or zebrafish embryos were harvested in TRIZOL (Invitrogen), treated with DNase I (Ambion) and single-stranded cDNA synthesized from 1 μg total RNA using superscript III (Invitrogen) and random hexamers. qPCR was performed on an ABI7300 (Applied Biosystems) using Sybergreen for detection. All samples were analyzed in duplicate. Analysis utilized ABI7300 system software ver1.3.1 and threshold levels were set during the exponential phase of amplification. Delta Cₜ relative quantification, PCR efficiency correction and multiple reference normalization (UBC, HPRT, HMBS, YWHAZ) were calculated using qBase²⁵.

The applicant has surprisingly discovered that although the ORF of WTX is annotated as lying within a single exon, detection of the expression of WTX using RT-PCR identifies an alternatively spliced transcript that excludes residues 50-327 (FIGS. 2, 4a) including the nuclear localisation sequence, most of APCBD1 and at least one predicted binding site for PtdIns(4,5)P2. Consideration of the mechanisms governing nonsense-mediated mRNA decay predicts that transcripts with a truncating mutation within the single exon spliceform (WTXS1) or lying within the intron of spliceform 2 (WTXS2), will not be subject to degradation. Accordingly, the applicant has detected both isoforms—the full-length (WTXS1) and the spliced, shorter version (WTXS2) were using qRT-PCR on cDNA from lymphoblastoid cell lines obtained from an affected surviving male with a mutation in exon 2 of WTXS2 (case 4) at levels similar to those observed in cell lines obtained from several healthy controls (FIG. 4a, b).

In addition, the applicant has found that expression of full-length N-terminal FLAG-tagged genomic WTX and genomic constructs containing the mutation c.671delC (associated with male lethality) in HEK293 cells identifies two proteins with sizes consistent with translation of the two alternatively spliced transcripts (FIG. 4b).

These results indicate that the truncated proteins encoded by sequences 5′ to nonsense mutations are produced in the cells of affected individuals with OSCS and that these peptides retain some properties of full-length WTX, including the ability to bind β-catenin and/or APC. These retained functions modify the resultant phenotype and serve to explain the genotype-phenotype correlation that relates the position of the mutations in WTX with survivability in males.

These results provide strong evidence that the location of mutations in males with OSCS predicts the structure of spliceforms associated with lethal and non-lethal male phenotypes. Mutations resulting in lethal phenotypes are associated with the production of WTX_{S1} variants that do not retain the full length APCBD1, the putative NLS or at least one PtdIns(4,5)P₂ binding domain in conjunction with the acidic domain (AD) (FIG. 5). These same alleles are however predicted to retain the capability to produce full length WTXS2. Alleles associated with survivorship predict a WTXS1 isoform that retains APCBD1, NLS and PtdIns(4,5)P₂ domains in addition to the capacity to bind β-catenin. The shorter WTXS2 isoform however has a C-terminal truncation either prior to or within APCBD2.

The applicant tested the prediction of retained functions for certain truncated WTX isoforms by co-immunoprecipitation experiments from lysates of cells cotransfected with a FLAG-tagged construct expressing full-length WTX and β-catenin. Both WTXS1 and WTXS2 isoforms retain the ability to bind β-catenin (FIG. 4c). Additionally immunostaining of HEK293 cells transfected with FLAG-tagged WTXS1 and WTXS2 constructs demonstrated predominantly plasma membrane localisation of WTXS1 whilst WTXS2 exhibits a more diffuse cytoplasmic distribution. The applicant has determined from these results, the localisation of the Nterm PtdIns(4,5)P₂ binding sites to within the intron of WTXS2 (FIG. 4d). These data indicate that β-catenin binding capability requires protein domains that are excluded in WTXS2, such as the PtdIns(4,5)P₂ binding sites and/or the nuclear localization sequence, to confer survivability in the male. Both mutations conferring milder male phenotypes (p.Y425X and p.L502fs) predict WTX proteins truncated within APCBD2, an observation that implies C-terminal domains in WTX, possibly APCBD2 and APCBD3, also contribute to regulation of Wnt signaling or other WTX functions related to bone mass accrual.

The applicant's discovery is consistent with the more marked skeletal phenotype observed females with whole gene deletions (cases 06 and 07).

### Example 4

### Germline WTX Mutations do not Increase Cancer Disposition

The applicants has identified that individuals with germline mutations in WTX do not show an increased disposition to tumorigenesis. The applicants report the results of studies carried out on 25 individuals with germline mutations in OSCS (Table 2). Table 2 summarizes mutations in WTX, X-inactivation analysis, and clinical phenotypes associated with OSCS in males and females. The right hand column refers to their cancer history. As can be seen from the data, only 1/25 have had any malignancy. The age range of the individuals reported is from fetus to 55 years of age.

TABLE 2

| | | | | | | X- | | | | | | | | |
|---|---|---|---|---|---|---|---|---|---|---|---|---|---|---|
| | | | | | | inacti- | | Develop- | Oro- | | | | | History |
| | | | | | | vation | | mental | facial | Cardiac | Deaf- | Sclerosis | Stria- | of |
| Case | Age | | Sex | DNA | Protein | ratio^{§} | de novo/familial | delay | clefting | anomaly | ness | of bones | tions | cancer |
| 01 | fetus | | M | c.671delC | p.P224fs+57X | — | inherited from 11 | U | − | + | U | + | − | − |
| 02 | fetus | | M | c.780insA | p.P260fs+16X | — | inherited from 12 | U | + | − | U | + | − | − |
| 03 | fetus | | M | c.1072C>T* | p.R358X | — | de novo | U | + | − | U | + | − | − |
| 04 | 17 | y | M | c.1275C>G | p.Y425X | — | inherited from 23 | + | − | + | + | + | − | − |
| 05 | 24 | y | M | c.1506delA | p.G502fs+38X | — | inherited from 24 | + | − | − | − | + | − | − |
| 06 | 4 | y | F | gene deletion* | nil | 49:51 | de novo | + | + | + | + | ++ | − | − |
| 07 | 5 | y | F | gene deletion* | nil | 50:50 | unknown | + | + | − | + | ++ | − | − |
| 08 | 50 | y | F | c.502_3delGGinsT | p.F168fs+1X | 69:31 | unknown | − | − | − | − | + | + | − |
| 09 | 14 | y | F | c.502_3delGGinsT | p.F168fs+1X | 62:38 | inherited from 08 | − | + | − | − | + | + | − |
| 10 | 5 | y | F | c.637C>T | p.Q213X | 51:49 | de novo | + | + | − | + | + | + | − |
| 11 | 39 | y | F | c.671delC | p.P224fs+57X | UI | de novo | − | − | − | + | + | + | − |
| 12 | 35 | y | F | c.780insA | p.P260fs+16X | 77:23 | de novo | − | − | − | − | + | + | − |
| 13 | 2 | m | F | c.780insA | p.P260fs+16X | 66:34 | inherited from 12 | U | + | − | U | + | − | − |
| 14 | 55 | y | F | c.867_8delAG | p.T289fs+33X | 58:42 | unknown | + | − | − | − | + | + | − |
| 15 | 28 | y | F | c.867_8delAG | p.T289fs+33X | 71:29 | inherited from 14 | + | − | − | + | + | + | − |
| 16 | 25 | y | F | c.867_8delAG | p.T289fs+33X | 68:32 | inherited from 14 | + | + | − | + | + | + | − |
| 17 | 12 | y | F | c.1057C>T* | p.R353X | 81:19 | de novo | − | + | + | + | + | + | − |
| 18 | 36 | y | F | c.1057C>T* | p.R353X | | | − | − | − | + | + | + | − |
| 19 | 56 | y | F | c.1072C>T* | p.R358X | 60:40 | unknown | + | + | + | − | + | + | − |
| 20 | 35 | y | F | c.1072C>T* | p.R358X | 86:14 | de novo | − | + | − | + | + | + | − |
| 21 | 11 | y | F | c.1072C>T* | p.R358X | 68:32 | de novo | + | + | + | U | + | + | − |
| 22 | 11 | y | F | c.1072C>T* | p.R358X | 69:31 | de novo | + | + | − | + | + | + | − |
| 23 | 46 | y | F | c.1275C>G | p.Y425X | 21:79 | unknown | − | − | − | − | + | + | − |
| 24 | 55 | y | F | c.1506delA | p.G502fs+38X | 40:60 | unknown | − | − | − | − | + | + | CRC |
| 25 | 37 | y | F | c.1637insT | p.F545fs+34X | | unknown | − | − | − | + | + | + | − |

*Previously reported as a somatic mutation in Wilms tumor.
Y years, m months, U unknown, UI uninformative, M male, F female, CRC colorectal carcinoma, + feature present, − feature not present, § mean of three independent experiments.

### Example 5

### Identification and Effect of Anti-WTX siRNA(s)

Two siRNAs that inhibit WTX function were designed as follows:

Oligonucleotide and siRNA numbering is based on the Genbank entry NM_{—}175179 which includes 256 nt of 5′ untranslated region.

siRNA molecules (25 nt, double stranded) were obtained from Invitrogen (Carlsbad, Calif.), making use of the BLOCK-iT design tool available on the Invitrogen web site. Effectiveness of each siRNA was tested in mouse mesenchymal stem cell line C2C12. In a 24 well plate, cells were seeded at 50,000 cells per well in 0.5 ml of growth medium (Dulbecco's Modified Eagle's Medium with 10% fetal calf serum). SiRNAs were introduced using RNAiMAX transfection reagent. Per well quantities of material were 1.5 μl of RNAiMAX, 30 pmoles of siRNA, and 100 μl of Optimem transfection medium. Cells were harvested 24 hrs post-transfection in TRIZOL reagent (Invitrogen; 200 μl per well), from which the aqueous phase containing RNA was separated by addition of chloroform (40 μl per sample). RNA was precipitated by addition of linear polyacrylamide (20 μg per well) and 2-isopropanol (100 μl per sample). Pellets were washed in 70% ethanol, dried briefly, and resuspended in 10.5 μl of H₂O and reverse transcriptase primer mix. The primer mix was comprised of 0.25 μl of 20 μM oligo-dT and 0.25 μl of 10 μM of a reverse primer specific for the 3′ end of the murine Wtx transcript, ensuring priming of general mRNA, with enhanced detection of mouse Wtx transcripts. SuperscriptIII reverse transcriptase (0.125 μl per sample) and other reaction components were added for a total volume of 20 μl, and cDNA synthesis reactions were incubated at 50° C. for 1 hour. Samples were then diluted with 80 μl of H₂O and stored at −20° C.

Quantitative PCR was carried out using iQ Syber Green Mix (BioRad, Hercules, Calif.) in 25 μl reactions with 5 μl of cDNA template. Primers pairs were designed to amplify only from cDNA by targeting different exons. Reference genes that were used for normalisation were: CREB (cyclic AMP response element binding protein), HMBS (hydroxymethylbilane synthase) and SDHA (succinate dehydrogenase complex, subunit A).

Primers targeting mouse WTX and CREB were validated using purified PCR product, serially diluted over 7 orders of magnitude. Amplification and data acquisition was carried out using a 7300 RealTime PCR System (Applied Biosystems, Foster City, Calif.). Further analysis was carried out with qBasePlus (Biogazelle, Zulte, Belgium).

PCR reactions began with a 10 minute 95° C. denaturation/activation step, followed by 40 cycles of denaturation (95° C. for 10 seconds)/annealing-extension (annealing temperature for 1 minute). Annealing temperatures were 68° C. for WTX and CREB, and 60° C. for SDHA and HMBS.

Using CREB, SDHA and HMBS as reference genes, two WTX-targeting siRNAs were found to lower WTX mRNA levels by >75% relative to levels in untransfected cells and cells transfected with scrambled control siRNAs. These efficacious RNAs were MWTX446 and MWTX1907 and their cognate (scrambled) controls were MWTX446C and MWTX1907C. Their sequences are: (sense strand)

| | |
|---|---|
| MWTX446: | |
| AGGGAAUCUGUACUCUGCCUAGUUU | (SEQ ID NO: 45) |
| MWTX1907: | |
| GCAACUCAGAGAUGUUGGAUCCUUU | (SEQ ID NO: 47) |
| MWTX446C: | |
| AGGUCUACAUGGUCUAUCCGAGUUU | (SEQ ID NO: 49) |
| MWTX1907C: | |
| GCAACUGAGGUAGUUUAGCCACUUU | (SEQ ID NO: 51) |

The applicants determined that two different siRNAs designated 1907 and 446 target and reduce the expression of WTX mRNA as follows:

Levels of WTX expression were measured by RT-qPCR performed on cDNA prepared from cells transfected with each of these siRNAs. RT qPCR was performed as known in the art and previously described herein. The ability of siRNAs 1907 and 446 to inhibit the expression and function of WTX mRNA in C2C12 cells was confirmed (FIG. 6).

The effects of siRNA inhibition of WTX expression were then measured by determining the effect of siRNAs 1907 and 446 on canonical Wnt signaling. Published results (Major et al., 2007) indicate that WTX functions to activate canonical Wnt signaling in transfected cells with consequent transcriptional up-regulation of Wnt responsive gene targets.

The applicants therefore compared the ability of siRNAs 1907 and 446 to de-repress Wnt signaling in C2C12 mesenchymal stem cells compared to other established repressors, namely LiCl. The methodology for this experiment was:

Mouse C2C12 cells, a mesenchymal stem cell line in which an siRNA-mediated 80% knock-down of endogenous WTX mRNA had been achieved, were transfected with reporter plasmids, with and without siRNA. Co-transfections were carried out in 24 well plates. Cells were plated at a density of 50000 cells per well, in 0.5 ml of DMEM+10% FCS.

Separate transfection mixes were prepared for DNA and siRNA. The Wnt/β-catenin-responsive reporter plasmid TOPFLASH was used along with a β-galactosidase expressing plasmid pdx11-LacZ as a control (90 ng and 10 ng per well respectively). The transfection reagent was Lipofectamine2000 (Invitrogen, Carlsbad, Calif.; 0.25 μl per well), and DNA/Lipofectamine2000 complexes were prepared in Optimem (Invitrogen, 20 μl per well) as per manufacturer's instructions. For siRNA, 30 pmoles per well of MWTX1907 or MWTX1907C were diluted in 100 μl of Optimem along with 1.5 μl of RNAiMAX reagent (Invitrogen). Plasmid and siRNA transfection mixes were added to wells soon after plating, while cells were still in suspension. Triplicate wells were used for each treatment. The “No SiRNA” wells did not receive any siRNA or RNAiMAX, or additional Optimem. LiCl stimulation was carried out 20 hours post-transfection by adding 0.01 volumes per well of 2M LiCl. Cells were harvested in 100 μl per well cell lysis buffer (Promega, Madison, Wis.) 24 hrs later.

Luciferase activity was assayed using the Promega firefly luciferase assay system in a Synergy2 multi-mode plate reader (BioTek, Winooski, Vt.), and β-galactosidase was assayed using an O-nitrophenyl-β-D-galactopyranoside-based colorimetric assay (Sigma). The β-galactosidase values, which did not vary substantially between treatment groups, were used to normalise the luciferase values.

Our results (FIG. 11) indicate that the ability of WTX to exert de-repression of canonical Wnt transcriptional targets is modest, consistent with previously published results (Major et al.⁴). The applicants can conclude from their results that the (modest de-repression of canonical Wnt signaling by siRNAs that down-regulate the expression of WTX may not be the mechanism responsible for the pro-osteogenic effects observed in cells exhibiting reduced WTX expression and function. Rather, the pro-osteogenic effects observed in such cells appear to be un-related to the canonical Wnt signaling pathway, as expressed by transactivation of the TOPFLASH reporter construct.

Stated another way, these experiments provide evidence that canonical Wnt signaling to the TOPFLASH reporter within the nucleus does not itself appear to be a good measure of WTX function. The applicants' data also provide evidence that the pro-osteogenic action of inhibition of WTX function may be fundamentally different from other members of the Wnt signaling pathway in their anabolic action on bone.

### Example 6

### To Show that Inhibition of WTX Expression in Human Mesenchymal Stem Cells Accelerates the Appearance of Enzymatic Indices Indicative of Osteoblast Differentiation

In order to show that inhibition of WTX expression in human mesenchymal stem cells accelerates the appearance of enzymatic indices indicative of osteoblast differentiation the following experiments, or similar experiments well known to those skilled in the art, may be performed. WTX expression may be, for example, inhibited in human cells having different pluripotential cell differentiation states (human mesenchymal stem cells). Osteoblast differentiation may then be determined by measurement of the altered gene expression of certain osteogenic genes, responsive to WTX gene expression. Suitable gene markers of osteoblast differentiation can include those whose altered expression is indicative of increased osteoblast differentiation and include, but are not limited to, osteocalcin OC; osteonectin ON; osterix OX; alkaline phosphatase ALP; collagen 1 COL1A1 and COL1A2. Altered gene expression may be measured using RT-qPCR as described herein using cDNAs of osteoblast gene products obtained from human mesenchymal stem cells that show reduced WTX gene expression due to inhibition using siRNAs 1907 and 446, by measuring enzymatic activity directly or by direct measurement of calcium phosphate production or by any suitable combinations of the above. The expression of these osteoblast gene markers is representative of the stages of development/differentiation and/or the different proliferation phases of osteoblast maturation.

Method for hMSC Culture and Osteogenic Differentiation

Human mesenchymal stem cells (hMSC) may be obtained from Lonza, Walkersville, Md., USA. The cells can be cultured in MSCGM (Lonza, # PT-3001; Mesenchymal stem cell basal medium supplemented with SingleQuots containing MCGS, L-Glutamine and GA-1000) at 37° C. in 5% CO₂ and passaged according to the manufacturer's instructions. Cells can be used between passages 4-6 for osteogenic differentiation.

hMSCs can be plated at a density of 31×10³ cells per well in a 6 well culture plate in MSCGM. After 24 hours the medium may be changed to osteogenic differentiation media (Lonza, #PT-3002: Differentiation basal medium supplemented with SingleQuots containing Dexamethasone, L-Glutamine, Ascorbate, MCGS, β-Glycerophosphate, and GA-1000), the medium can be changed every 3-4 days for up to 21 days, as required.

Method for qRT-PCR Analysis of Osteogenesis

RNA can be extracted from hMSC undergoing osteogenic differentiation by in well cell lysis in Trizol Reagent (Invitrogen). RNA can be extracted as per the manufacturer's instruction up to the point where RNA is separated into the aqueous phase. Thereafter, an equal volume of 70% ethanol can be added and the sample further purified on a PureLink Micro- to Midi total RNA purification column (Invitrogen) as per the manufacturer's instructions, and subsequently treated with DNA free (Ambion) DNase.

Reverse transcription can be carried out using SuperScript III Reverse Transcriptase (Invitrogen) with 500 ng total RNA, 50 uM oligo(dT)18 and 100 ng of random hexamer primer. Osteogenesis can be tracked by q-RTPCR of 5 osteogenic marker genes, COL1A1, RUNX2, APLP, OCN and SP7. Primers may be designed by primerdepot.nci.nih.gov, and q-RTPCR carried out using iQ SYBR Green Supermix (BioRad) on a ABI 7300 Real Time PCR System.

### Example 7

### To Show that Inhibition of WTX Gene Expression Leads to Improved Bone Healing

In order to show that inhibition of WTX gene expression leads to improved bone healing, for example, by increase in bone growth or bone density, in vivo, in rats, the following experiments, or similar experiments well known to those skilled in the art, may be performed. Any suitable rat model may be used, for example, Lewis rats, which are a genetically inbred strain derived from the Wistar strain. Rats are an art accepted animal model system for this purpose^{41,42}. A critical sized defect (defined as a bony lesion that will not spontaneously heal due to its size) of 8 mm in diameter may be sited in the rat cranium. One method for establishment of this model is that after anaesthesia, the rats can be immobilized on their abdomen, and the skull shaved. A semi-lunar incision may then be made from the nasal bone to the occipital protuberance. A full-thickness bone defect including the overlying periosteum can be created in the dorsal part of the parietal cranium central to the sagittal suture with a trephine bur under continuous saline irrigation with an outside diameter of 8 mm. Care should be taken not to damage the underlying dura mater and sagittal sinus. After removing the bone segment, 80 ul of siRNA (either or both of the therapeutic siRNAs 1907 and 446), dispersed using any of a variety of vehicles, including, but not limited to a pluronic acid gel, may be directly placed on the lesions. The skin of the cranium can then be closed over the lesions using standard surgical techniques as known in the art. Histological examination of the treated lesions may be performed at several time intervals spanning from 3 days post procedure, up to at least one month (to detect the presence of primary osteoid deposition), and three months post procedure (to quantify the amount of new bone formation). Rats sacrificed at these timepoints may have their craniums removed and these specimens then subjected to combinations of histomorphometry, immunohistochemistry and to microCT analysis (the latter technique can allow direct quantification of new bone formation.

For microCT the specimens can be wrapped in cling film to prevent drying during scanning. Samples can then be analysed at an energy of 100 kV. Three-dimensional images can be created and evaluated for the percentage of defect closure. Subsequent analysis may volumetrically quantify the degree of defect closure by defining a volume of interest (VOI); specified as a cylindrical area covering the created defect with a diameter of 8.1 mm and depth of 1.2 mm). In this area, bone volume can be determined and expressed as a percentage of the VOI.

It is the applicants' expectation that siRNA mediated WTX repression will lead to improved bone healing, particularly faster bone healing. The applicants also anticipate that using the methods of the invention, the volume of bone accrued will be significantly greater in treated rats as compared to control rats treated only with a scrambled non-functional siRNA.

The above examples illustrate practice of the invention. It will be appreciated by those skilled in the art that numerous variations and modifications may be made without departing from the spirit and scope of the invention.

In the description in this specification reference may be made to subject matter which may not be within the scope of the claims of the current application. That subject matter should be readily identifiable by a person skilled in the art and may assist in putting into practice the invention as defined in the claims of this application.

Those skilled in the art will of course appreciate that the above description is provided by way of example and that the invention is not limited thereto.

### REFERENCES

1. Hurt, R. L. Osteopathia striata-Voorhoeve's disease; report of a case presenting the features of osteopathia striata and osteopetrosis. J Bone Joint Sugg Br 35-B, 89-96 (1953).
2. Pellegrino, J. E., McDonald-McGinn, D. M., Schneider, A., Markowitz, R. I. & Zackai, E. H. Further clinical delineation and increased morbidity in males with osteopathia striata with cranial sclerosis: an X-linked disorder? Am J Med Genet 70, 159-65 (1997).
3. Nannya, Y. et al. A robust algorithm for copy number detection using high density oligonucleotide single nucleotide polymorphism genotyping arrays. Cancer Res 65, 6071-9 (2005).
4. Major, M. B. et al. Wilms tumor suppressor WTX negatively regulates Wnt/betacatenin signaling. Science 316, 1043-6 (2007).
5. Bennett, C. N. et al. Wnt10b increases postnatal bone formation by enhancing osteoblast differentiation. J Bone Miner Res 22, 1924-32 (2007).
6. Morvan, F. et al. Deletion of a single allele of the Dkk1 gene leads to an increase in bone formation and bone mass. J Bone Miner Res 21, 934-45 (2006).
7. Li, X. et al. Sclerostin binds to LRP5/6 and antagonizes canonical Wnt signaling. J Biol Chem 280, 19883-7 (2005).
8. Little, R. D. et al. A mutation in the LDL receptor-related protein 5 gene results in the autosomal dominant high-bone-mass trait. Am J Hum Genet 70, 11-9 (2002).
9. Clement-Lacroix, P. et al. Lrp5-independent activation of Wnt signaling by lithium chloride increases bone formation and bone mass in mice. Proc Natl Acad Sci USA 102, 17406-11 (2005).
10. Rivera, M. N. et al. An X chromosome gene, WTX, is commonly inactivated in Wilms tumor. Science 315, 642-5 (2007).
11. Grzeschik, K. H. et al. Deficiency of PORCN, a regulator of Wnt signaling, is associated with focal dermal hypoplasia. Nat Genet 39, 833-5 (2007).
12. Wang, X. et al. Mutations in X-linked PORCN, a putative regulator of Wnt signaling, cause focal dermal hypoplasia. Nat Genet 39, 836-8 (2007).
13. Ruteshouser, E. C., Robinson, S. M. & Huff, V. Wilms tumor genetics: Mutations in WT1, WTX, and CTNNB1 account for only about one-third of tumors. Genes Chromosomes Cancer 47, 461-470 (2008).
14. Grohmann, A., Tanneberger, K., Alzner, A., Schneikert, J. & Behrens, J. AMER1 regulates the distribution of the tumor suppressor APC between microtubules and the plasma membrane. J Cell Sci 120, 3738-47 (2007).
15. Gaur, T. et al. Canonical Wnt signaling promotes osteogenesis by directly stimulating Runx2 gene expression. J Biol Chem 280, 33132-40 (2005).
16. Holmen, S. L. et al. Essential role of beta-catenin in postnatal bone acquisition. J Biol Chem 280, 21162-8 (2005).
17. Hill, T. P., Taketo, M. M., Birchmeier, W. & Hartmann, C. Multiple roles of mesenchymal beta-catenin during murine limb patterning. Development 133, 1219-29 (2006).
18. Zhou, H., Mak, W., Zheng, Y., Dunstan, C. R. & Seibel, M. J. Osteoblasts directly control lineage commitment of mesenchymal progenitor cells through Wnt signaling. J Biol Chem 283, 1936-45 (2008).
19. Weinberg, R. A. Tumor suppressor genes. Science 254, 1138-46 (1991).
20. Futreal, P. A. et al. A census of human cancer genes. Nat Rev Cancer 4, 177-83 (2004).
21. Royer-Pokora, B. et al. Twenty-four new cases of WT1 germline mutations and review of the literature: genotype/phenotype correlations for Wilms tumor development. Am J Med Genet A 127, 249-57 (2004).
22. Allen, R. C., Zoghbi, H. Y., Moseley, A. B., Rosenblatt, H. M. & Belmont, J. W. Methylation of HpaII and HhaI sites near the polymorphic CAG repeat in the human androgen-receptor gene correlates with X chromosome inactivation. Am J Hum Genet 51, 1229-39 (1992).
23. Maiti, S., Alam, R., Amos, C. I. & Huff, V. Frequent association of beta-catenin and WT1 mutations in Wilms tumors. Cancer Res 60, 6288-92 (2000).
24. Livak, K. J. & Schmittgen, T. D. Analysis of relative gene expression data using real-time quantitative PCR and the 2(-Delta Delta C(T)) Method. Methods 25, 402-8 (2001).
25. Hellemans, J., Mortier, G., De Paepe, A., Speleman, F. & Vandesompele, J. qBase relative quantification framework and software for management and automated analysis of real-time quantitative PCR data. Genome Biol 8, R19 (2007).
26. Logan et al. ANN Rev. Cell Dev. Biol 20: 781 (2004)
27. Willert et al. Genes Dev. 20: 1394 (2006))
28. Harlow and Lane 1998. Antibodies: A Laboratory Manual, Cold Spring Harbour Press New York.²⁷
29. Kohler and Milstein 1975. Continuous Cultures of Fused Cells Secreting Antibody of Predefined Specificity. Nature, 256, 495-497.
30. Michael Neuberger (1996) Generating high-avidity human Mabs in mice Nature Biotechnology 14, 826
31. Tristan J. Vaughan, Jane K. Osbourn & Philip R. Tempest (1998) Human antibodies by design. Nature Biotechnology 16, 535-539
32. Milstein and Cuello (1983) The co-expression of two immunoglobulin heavy-chain/light-chain pairs, where the two heavy chains have different specificities, Nature, 305:537-539.
33. Suresh, M. R., Cuello, A. C. and Milstein, C. (1986) Bi-specific monoclonal antibodies from hybrid hybridomas. Methods in Enzymology, 121: 210-228.
34. Brennan et al., “Preparation of bispecific antibodies by chemical recombination of monoclonal immuno globulin G1 fragments” Science 229:81-83 (1985).
35. Hunt P J, Richards A M, Nicholls M G, Yandle T G, Doughty R N, Espiner E A. Immunoreactive amino terminal pro brain natriuretic peptide (NT-proBNP): a new marker of cardiac impairment. Clin. Endocrinol. 1997 47:287-296.
36. Kim S H, Cech T R. Three-dimensional model of the active site of the self-splicing rRNA precursor of Tetrahymena. Proc Natl Acad Sci USA. 1987 December; 84(24):8788-92.
37. Forster A C, Symons R H. Self-cleavage of virusoid RNA is performed by the proposed 55-nucleotide active site. Cell. 1987 Jul. 3, 50(1):9-16.
38. Joyce G F. Amplification, mutation and selection of catalytic RNA. Gene. 1989 Oct. 15, 82(1):83-7
39. Cech T R, Zaug A J, Grabowski P J. In vitro splicing of the ribosomal RNA precursor of Tetrahymena: involvement of a guanosine nucleotide in the excision of the intervening sequence. Cell. 1981 Dec., 27 (3 Pt 2): 847-96.
40. Pang E K, Im S U, Kim C S, Choi S H, Chai J K, Kim C K, Han S B, Cho K S. Effect of recombinant human bone morphogenetic protein-4 dose on bone formation in a rat calvarial defect model. J Periodontol. 2004 October; 75(10):1364-70.
41. Blum J S, Barry M A, Mikos A G, Jansen J A. In vivo evaluation of gene therapy vectors in ex vivo-derived marrow stromal cells for bone regeneration in a rat critical-size calvarial defect model. Hum Gene Ther. 2003 Dec. 10; 14(18):1689-701.

All references and citations in this list and throughout the specification including patent specifications are hereby incorporated in their entirety.

### SUMMARY OF SEQUENCE LISTING

| Human wild type WTX cDNA | | (SEQ ID NO: 1) |
|---|---|---|
| Human wild type WTX amino acid | | (SEQ ID NO: 2) |
| Human S2 splice variant WTX cDNA | | (SEQ ID NO: 3) |
| Human S2 splice variant WTX amino acid | | (SEQ ID NO: 4) |
| Truncated WTX 224fs cDNA | | (SEQ ID NO: 5) |
| Truncated WTX 224fs amino acid | | (SEQ ID NO: 6) |
| Truncated WTX 260fs cDNA | | (SEQ ID NO: 7) |
| Truncated WTX 260fs amino acid | | (SEQ ID NO: 8) |
| Truncated WTX R358X cDNA | | (SEQ ID NO: 9) |
| Truncated WTX R358X amino acid | | (SEQ ID NO: 10) |
| Truncated WTX Y425X cDNA | | (SEQ ID NO: 11) |
| Truncated WTX Y425X amino acid | | (SEQ ID NO: 12) |
| Truncated WTX 502fs cDNA | | (SEQ ID NO: 13) |
| Truncated WTX 502fs amino acid | | (SEQ ID NO: 14) |
| Truncated WTX c.502_3delGGinsT cDNA | | (SEQ ID NO: 15) |
| Truncated WTX p.F173fs+1X amino acid | | (SEQ ID NO: 16) |
| Truncated WTX c.637C>T cDNA | | (SEQ ID NO: 17) |
| Truncated WTX p.Q213X | | (SEQ ID NO: 18) |
| Truncated WTX c.867_8delAG cDNA | | (SEQ ID NO: 19) |
| Truncated WTX p.T289fs+33X | | (SEQ ID NO: 20) |
| Truncated WTX c.1057C>T cDNA | | (SEQ ID NO: 21) |
| Truncated WTX p.R353X | | (SEQ ID NO: 22) |
| Truncated WTX c.1637insT cDNA | | (SEQ ID NO: 23) |
| Truncated WTX p.F545fs+33X | | (SEQ ID NO: 24) |
| Mouse wild type WTX cDNA | | (SEQ ID NO: 25) |
| Mouse wild type WTX amino acid | | (SEQ ID NO: 26) |
| Mouse S2 splice variant WTX cDNA | | (SEQ ID NO: 27) |
| Mouse S2 splice variant WTX amino acid | | (SEQ ID NO: 28) |
| Sense strand | WTX-A | (SEQ ID NO: 29) |
| Antisense strand | WTX-A | (SEQ ID NO: 30) |
| Sense strand | WTX-B | (SEQ ID NO: 31) |
| Antisense strand | WTX-B | (SEQ ID NO: 32) |
| Pan troglodytes WTX cDNA | | (SEQ ID NO: 33) |
| Pan troglodytes WTX amino acid | | (SEQ ID NO: 34) |
| Canis familiaris WTX cDNA | | (SEQ ID NO: 35) |
| Canis familiaris WTX amino acid | | (SEQ ID NO: 36) |
| Bos taurus WTX cDNA | | (SEQ ID NO: 37) |
| Bos taurus WTX amino acid | | (SEQ ID NO: 38) |
| Rattus norvegicus WTX cDNA | | (SEQ ID NO: 39) |
| Rattus norvegicus WTX amino acid | | (SEQ ID NO: 40) |
| Gallus gallus WTX cDNA | | (SEQ ID NO: 41) |
| Gallus gallus WTX amino acid | | (SEQ ID NO: 42) |
| Danio rerio WTX cDNA | | (SEQ ID NO: 43) |
| Dania rerio WTX amino acid | | (SEQ ID NO: 44) |
| Sense strand | MWTX446 | (SEQ ID NO: 45) |
| Antisense strand | MWTX446 | (SEQ ID NO: 46) |
| Sense strand | MWTX1907 | (SEQ ID NO: 47) |
| Antisense strand | MWTX1907 | (SEQ ID NO: 48) |
| Sense strand | MWTX446c | (SEQ ID NO: 49) |
| Antisense strand | MWTX446c | (SEQ ID NO: 50) |
| Sense strand | MWTX1907c | (SEQ ID NO: 51) |
| Antisense strand | MWTX1907c | (SEQ ID NO: 52) |

### Further Sequence Information:

SEQ ID NO: 1—WTX S1 polynucleotide (>gi|124244055|ref|NM_{—}152424.3| Homo sapiens family with sequence similarity 123B (FAM123B), cDNA)
SEQ ID NO: 2—WTX S1 amino acid (>gi|124244056|ref|NP_{—}689637.3| family with sequence similarity 123B [Homo sapiens])
SEQ ID NO: 3—Human WTX S2 cDNA (polynucleotide)
SEQ ID NO: 4—WTXS2 amino acid (family with sequence similarity 123B [Homo sapiens] Spliceform S2)
SEQ ID NO: 5—c.671delC polynucleotide
SEQ ID NO: 6—p.P224fs+57X (amino acid)
SEQ ID NO: 7—c.780insA (polynucleotide)
SEQ ID NO: 8—p.P260fs+16X(amino acid)
SEQ ID NO: 9—c.1072C>T (polynucleotide)
SEQ ID NO: 10—p.R358X (amino acid)
SEQ ID NO: 11—c.1275C>G (polynucleotide)
SEQ ID NO: 12—p.Y425X (amino acid)
SEQ ID NO: 13—c.1506delA (polynucleotide)
SEQ ID NO: 14—p.G502fs+38X (amino acid)
SEQ ID NO: 15—c.502_{—}3delGGinsT cDNA (polynucleotide)
SEQ ID NO: 16—p.F173fs+1X (amino acid)
SEQ ID NO: 17—c.637C>T cDNA (polynucleotide)
SEQ ID NO: 18—p.Q213X (amino acid)
SEQ ID NO: 19—c.867_{—}8delAG cDNA (polynucleotide)
SEQ ID NO: 20—p.T289fs+33X (amino acid)
SEQ ID NO: 21—c.1057C>T cDNA (polynucleotide)
SEQ ID NO: 22—p.R353X (amino acid)
SEQ ID NO: 23—c.1637insT cDNA (polynucleotide)
SEQ ID NO: 24—p.F545fs+33X (amino acid)
SEQ ID NO: 25—>gi|123858769|ref|NM_{—}175179.3| Mus musculus RIKEN cDNA 2810002009 gene (2810002009Rik) (polynucleotide)
SEQ ID NO: 26—>gi|123858770|ref|NP_{—}780388.2| RIKEN cDNA 2810002009 [Mus musculus] (amino acid)
SEQ ID NO: 27—Mouse WTX cDNA S2 (polynucleotide)
SEQ ID NO: 28—Mouse WTX Protein S2 (amino acid)
SEQ ID NO: 29—WTXA siRNA sense strand (polynucleotide)
SEQ ID NO: 30—WTXA siRNA antisense strand (polynucleotide)
SEQ ID NO: 31—WTXB siRNA sense strand (polynucleotide)
SEQ ID NO: 32—WTXB siRNA antisense strand (polynucleotide)
SEQ ID NO: 33—>gi|114688895|ref|XM_{—}521092.2| PREDICTED: Pan troglodytes hypothetical LOC465669 (LOC465669), cDNA (polynucleotide)
SEQ ID NO: 34—>gi|114688896|ref|XP_{—}521092.2| PREDICTED: hypothetical protein [Pan troglodytes] (amino acid)
SEQ ID NO: 35—>gi|74007483|ref|XM_{—}843253.1| PREDICTED: Canis familiaris hypothetical protein LOC606822 (LOC606822), cDNA (polynucleotide)
SEQ ID NO: 36—>gi|74007484|ref|XP_{—}848346.1| PREDICTED: hypothetical protein XP_{—}843253 [Canis familiaris] (amino acid)
SEQ ID NO: 37—>gi|76678403|ref|XM_{—}584347.2| PREDICTED: Bos taurus hypothetical LOC539029 (LOC539029), cDNA (polynucleotide)
SEQ ID NO: 38—>gi|76678404|ref|XP_{—}584347.2| PREDICTED: hypothetical protein [Bos taurus] (amino acid)
SEQ ID NO: 39—>gi|109510850|ref|XM_{—}576986.2| PREDICTED: Rattus norvegicus similar to hypothetical protein FLJ39827 (predicted) (RGD1560322_predicted), cDNA (polynucleotide)
SEQ ID NO: 40—>gi|62666944|ref|XP_{—}576986.1| PREDICTED: hypothetical protein [Rattus norvegicus] (amino acid)
SEQ ID NO: 41—>gi|118089578|ref|XM_{—}420290.2| PREDICTED: Gallus gallus similar to hypothetical protein FLJ39827 (LOC422314), cDNA (polynucleotide)
SEQ ID NO: 42—>gi|118089579|ref|XP_{—}420290.2| PREDICTED: hypothetical protein [Gallus gallus ] (amino acid)
SEQ ID NO: 43—>gi|125815794|ref|XM_{—}001341920.1| PREDICTED: Danio rerio hypothetical protein LOC100002085 (LOC100002085), cDNA (polynucleotide)
SEQ ID NO: 44—>gi|125815795|ref|XP_{—}001341956.1| PREDICTED: similar to family with sequence similarity 123B [Danio rerio](amino acid)
SEQ ID NO: 45—MWTX446 siRNA sense strand (polynucleotide)
SEQ ID NO: 46—MWTX446 siRNA antisense strand (polynucleotide)
SEQ ID NO: 47—MWTX1907 siRNA sense strand (polynucleotide)
SEQ ID NO: 48—MWTX1907 siRNA antisense strand (polynucleotide)
SEQ ID NO: 49—MWTX446C siRNA sense strand (polynucleotide)
SEQ ID NO: 50—MWTX446C siRNA antisense strand (polynucleotide)
SEQ ID NO: 51—MWTX1907C siRNA sense strand (polynucleotide)
SEQ ID NO: 52—MWTX1907C siRNA antisense strand (polynucleotide)

### Accession Numbers for WTX Orthologues:

| Species | Gene | mRNA | Protein |
|---|---|---|---|
| H. sapiens | FAM123B | NM_152424.3 | NP_689637.3 |
| P. troglodytes | LOC465669 | XM_521092.2 | XP_521092.2 |
| C. familiaris | LOC606822 | XM_843253.1 | XP_848346.1 |
| B. taurus | LOC539029 | XM_584347.2 | XP_584347.2 |
| M. musculus | 2810002O09Rik | NM_175179.3 | NP_780388.2 |
| R. norvegicus | RGD1560322 | XM_576986.2 | XP_576986.1 |
| G. gallus | LOC422314 | XM_420290.2 | XP_420290.2 |
| D. rerio | LOC100002085 | XM_001341920.1 | XP_001341956.1 |

1-16. (canceled)

17. A method for treating a bone-related disease or condition in a subject, the method comprising administration to a subject in need thereof, of a therapeutically effective amount of an agent that modulates the expression or activity of a WTX polypeptide in the subject.

18. (canceled)

19. A method of promoting bone healing or bone formation, the method comprising contacting cells with an agent that modulates the expression or activity of a WTX polypeptide.

20. A method of modulating osteoprogenitor cell differentiation, the method comprising contacting osteoprogenitor cells with an agent that modulates the expression or activity of a WTX polypeptide.

21. A method for increasing or stimulating osteoblast differentiation, the method comprising administration to multipotent progenitor cells of an agent that modulates the expression or activity of a WTX polypeptide.

22. A method for stimulating or enhancing mineralization of matrix by cells, the method comprising administration to cells of an agent that modulates the expression or activity of a WTX polypeptide.

23. The method of any one of claims 17, 19, 20, 21 and 22 wherein the agent is selected from the group consisting of:
(i) an antisense compound that inhibits the expression of said WTX polypeptide,
(ii) an expression vector encoding said antisense compound,
(iii) a dsRNA that inhibits the expression of said WTX polypeptide,
(iv) one or more expression vectors encoding said dsRNA,
(v) a small molecule inhibitor of WTX gene expression or activity,
(vi) an aptamer that inhibits WTX gene expression or activity, and
(vii) a peptide nucleic acid that inhibits WTX gene expression or activity.

24. The method of any one of claims 17, 19, 20, 21 and 22, wherein the agent is formulated in a composition with a pharmaceutically acceptable carrier.

25. The method of any one of claims 17, 19, 20, 21 and 22, wherein said agent is an isolated siRNA or shRNA comprising a sense RNA strand and an antisense RNA strand, wherein the sense and the antisense RNA strands form an RNA duplex, and wherein the sense RNA strand comprises a nucleotide sequence identical to a target sequence of about 18 to about 25 contiguous nucleotides of a WTX mRNA.

26. The method of claim 25, wherein the target WTX mRNA sequence is a human WTX mRNA that comprises SEQ ID NO: 1 or a mouse WTX mRNA that comprises SEQ ID NO: 25.

27. (canceled)

28. The method of claim 26, wherein the siRNA or shRNA is selected from the group consisting of an siRNA or an shRNA having a sense strand that comprises SEQ ID NO: 45 and an antisense strand that comprises SEQ ID NO: 46 and an siRNA or shRNA having a sense strand that comprises SEQ ID NO: 47 and an antisense strand comprises SEQ ID NO: 48.

29. The method of any one of claims 17, 21 and 22, wherein the agent or the siRNA is administered in conjunction with a delivery reagent.

30. The method of claim 17, further comprising the administration of an additional therapeutic agent.

31. The method of any one of claims 17, 21 and 22, wherein said administration is oral administration, topical administration or parenteral administration.

32. (canceled)

33. (canceled)

34. (canceled)

35. (canceled)

36. (canceled)

37. (canceled)

38. A double-stranded RNA comprising the sequence of SEQ ID NO: 45 or SEQ ID NO: 47 or a complement thereof.

39. The double-stranded RNA of claim 38 that is no more than about 30 nucleotides in length.

40. The double-stranded RNA of claim 38 that is an shRNA or an siRNA.

41. The method of claim 23, wherein the agent is the double-stranded RNA comprising the sequence of SEQ ID NO: 45 or SEQ ID NO: 47 or a complement thereof.
